(19) United States
(12) Patent Application Publication
(10) Pub. No.: US 2021/0254101 A1
(43) Pub. Date: Aug. 19, 2021

(54) METHODS FOR TREATING SPINAL CORD INJURY

(71) Applicant: THE CHILDREN'S MEDICAL CENTER CORPORATION, BOSTON, MA (US)

(72) Inventors: ZHIGANG HE, BOSTON, MA (US); BO CHEN, BOSTON, MA (US)

(73) Assignee: THE CHILDREN'S MEDICAL CENTER CORPORATION, BOSTON, MA (US)

(21) Appl. No.: 17/058,046

(22) PCT Filed: May 21, 2019
(86) PCT No.: PCT/US2019/033303

§ 371 Date: Nov. 23, 2020

Related U.S. Application Data
(60) Provisional application No. 62/676,464, filed on May 25, 2018.

(51) Int. Cl. C12N 15/86; C12N 15/113

(52) U.S. Cl. CPC C12N 15/86; C12N 15/113; C12N 2310/141; C12N 2310/11; C12N 2750/14143

(57) ABSTRACT

Described herein are methods and compositions for treating a spinal injury. Aspects of the invention relate to administering to a subject an agent that upmodulates KCC2. Another aspect of the invention relates to administering to a subject an agent that that reduces excitability of inhibitory interneurons. Compositions comprising these agents are additionally described herein.

### CROSS REFERENCE TO RELATED APPLICATION

This application is an International Application which claims the benefit under 35 U.S.C. § 119(e) of U.S. Provisional Applications No. 62/676,464, filed on May 25, 2018, the contents of which is incorporated herein by reference in its entirety.

### FIELD OF THE INVENTION

The field of the invention relates to the treatment of spinal cord injuries.

### BACKGROUND

Many human spinal cord injuries are anatomically incomplete, but exhibit complete paralysis. It is unknown why spared axons fail to mediate functional recovery in these cases. Current therapeutics for such injury are limited, and often do not regenerate functional recovery of a spinal cord injury. Thus, a better understanding of axon regeneration is required for developing effective treatments.

### SUMMARY

The invention described herein is related, in part, to the discovery that an agent, e.g., CLP290, that upmodulates neuron-specific K⁺—Cl⁻ co-transporter (KCC2) activity and/or levels was capable of restoring stepping function in mice with staggered bilateral hemisections, e.g., a severe spinal cord injury model. Further, overexpression of KCC2 recapitulated this restoration of stepping. It is further shown herein that the inhibition of Na+/2Cl−/K+ co-transporter (NKCC) additionally restores stepping ability.

Further, work described herein show that agents that reduce excitability in interneurons in combination with clozapine N-oxide additionally restore the stepping ability in mice that have previously lost this ability following a staggered bilateral hemisection. Such agents include an agen that upmodulates a Gi-DREADD which has been optimized for expression in inhibitory interneurons, and Kir2.1.

Additionally, described herein are compositions comprising agent for modulating KCC2, NKCC, Gi-DREADD, and Kir2.1 to be used, e.g., in the treatment of a spinal cord injury.

Accordingly, one aspect of the invention described herein provides a method for treating a spinal injury, comprising administering to a subject having a spinal injury an effective amount of an agent that upmodulates KCC2.

In one embodiment of any aspect, the agent that upmodulates KCC2 is selected from the group consisting of a small molecule, a peptide, a gene editing system, and an expression vector encoding KCC2.

In one embodiment of any aspect, the small molecule is CLP290.

In one embodiment of any aspect, the vector is non-integrative or integrative. In another embodiment of any aspect, the vector is a viral vector or non-viral vector.

Exemplary non-integrative vectors include, but are not limited to, an episomal vector, an EBNA1 vector, a minicircle vector, a non-integrative adenovirus, a non-integrative RNA, and a Sendai virus.

Exemplary viral vectors include, but are not limited to, retrovirus, lentivirus, adenovirus, herpesvirus, poxvirus, alpha virus, vaccinia virus, and adeno-associated viruses.

Exemplary non-viral vectors include, but are not limited to, a nanoparticle, a cationic lipid, a cationic polymer, a metallic nanoparticle, a nanorod, a liposome, microbubbles, a cell penetrating peptide and a liposphere.

In one embodiment of any aspect, the vector crosses the blood brain barrier.

In one embodiment of any aspect, KCC2 is upmodulated by at least 2-fold, 5-fold, 6-fold, 7-fold, 8-fold, 9-fold, 10-fold as compared to an appropriate control.

In one embodiment of any aspect, the spinal injury is a severe spinal cord injury.

In one embodiment of any aspect, the subject is human. In one embodiment of any aspect, the subject has been diagnosed with a spinal injury. In one embodiment of any aspect, the subject has been previously diagnosed with a spinal injury. In one embodiment of any aspect, the subject has been previously treated for a spinal injury.

In one embodiment of any aspect, prior to administering, the subject is diagnosed with having a spinal cord injury.

In one embodiment of any aspect, the subject is further administered at least a second spinal injury treatment. In one embodiment of any aspect, the subject is further administered at least a second therapeutic compound. Exemplary second therapeutic compound include, but are not limited to osteopontin, growth factors, or 4-aminopuridine.

Another aspect of the invention described herein provides a method for treating a spinal injury, comprising administering to a subject having a spinal injury an effective amount of an agent that inhibits Na+/2Cl−/K+ co-transporter (NKCC).

In one embodiment of any aspect, the agent that inhibits Na+/2Cl−/K+ co-transporter (NKCC) is selected from the group consisting of a small molecule, an antibody, a peptide, an antisense oligonucleotide, and an RNAi. In one embodiment of any aspect, the RNAi is a microRNA, an siRNA, or an shRNA. In one embodiment of any aspect, the small molecule is bumetanide.

In one embodiment of any aspect, the agent is comprised in a vector.

Yet another aspect of the invention described herein provides a method for treating a spinal injury, comprising administering to a subject having a spinal injury an effective amount of an agent that reduces excitability of inhibitory interneurons.

In one embodiment of any aspect, the agent upmodulates the inhibitory Gi-coupled receptor Gi-DREADD.

In one embodiment of any aspect, the agent is an expression vector encoding Gi-DREADD. In one embodiment of any aspect, the agent is an expression vector encoding Kir2.1.

In one embodiment of any aspect, the method further comprises administering clozapine N-oxide at substantially the same time as the agent.

In one embodiment of any aspect, the excitability of inhibitory interneurons is reduced by at least 10%, at least 20%, at least 30%, at least 40%, at least 50%, at least 60%, at least 70%, at least 80%, at least 90, at least 99%, or more as compared to an appropriate control.

Another aspect of the invention described herein provides a method for treating a spinal injury, comprising administering to a subject having a spinal injury an effective amount electrical stimulation that reduces excitability of inhibitory interneurons. In one embodiment of any aspect, the method further comprises administering clozapine N-oxide.

In one embodiment of any aspect, the electrical stimulation is applied directly to the spinal cord. In one embodiment of any aspect, the electrical stimulation is applied directly to the spinal cord at the site of injury.

In one embodiment of any aspect, the excitability of inhibitory interneurons is reduced by at least 10%, at least 20%, at least 30%, at least 40%, at least 50%, at least 60%, at least 70%, at least 80%, at least 90, at least 99%, or more as compared to an appropriate control.

Another aspect of the invention described herein provides a pharmaceutical composition comprising an effective amount of KCC2 polypeptide or a vector comprising a nucleic acid sequence encoding the KCC2 polypeptide and a pharmaceutically acceptable carrier, for use in treating spinal cord injury.

In one embodiment of any aspect, the KCC2 polypeptide has, comprises, consists of, or consists essentially of at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more amino acid sequence identity to SEQ ID NO: 1 and retains at least 80% of the biological activity of KCC2 of SEQ ID NO: 1.

In one embodiment of any aspect, the composition further comprises at least a second therapeutic compound.

Another aspect of the invention described herein provides a pharmaceutical composition comprising an effective amount of Gi-DREADD polypeptide or a vector comprising a nucleic acid sequence the Gi-DREADD polypeptide and a pharmaceutically acceptable carrier, for use in treating spinal cord injury.

In one embodiment of any aspect, the Gi-DREADD polypeptide is an optimized Gi-DREADD polypeptide. In one embodiment of any aspect, the Gi-DREADD polypeptide comprises the sequence of SEQ ID NO: 2.

In one embodiment of any aspect, the Gi-DREADD polypeptide has, comprises, consists of, or consists essentially of at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more amino acid sequence identity to SEQ ID NO: 2 and retains at least 80% of the biological activity of Gi-DREADD of SEQ ID NO: 2.

In one embodiment of any aspect, the composition further comprises at least a second therapeutic compound. In one embodiment of any aspect, the composition further comprises clozapine N-oxide.

Another aspect of the invention described herein provides a pharmaceutical composition comprising an effective amount of Kir2.1 polypeptide or a vector comprising a nucleic acid sequence the Kir2.1 polypeptide and a pharmaceutically acceptable carrier, for use in treating spinal cord injury.

In one embodiment of any aspect, the Kir2.1 polypeptide comprises the sequence of SEQ ID NO: 3.

In one embodiment of any aspect, the Kir2.1 polypeptide has, comprises, consists of, or consists essentially of at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more amino acid sequence identity to SEQ ID NO: 3 and retains at least 80% of the biological activity of Kir2.1 of SEQ ID NO: 3.

In one embodiment of any aspect, the composition further comprises clozapine N-oxide. In one embodiment of any aspect, the composition further comprises at least a second therapeutic compound.

Another aspect of the invention described herein provides a pharmaceutical composition comprising an effective amount of any of the agents that inhibit NKCC as described herein and a pharmaceutically acceptable carrier, for use in treating spinal cord injury. In one embodiment of any aspect, the composition further comprises at least a second therapeutic compound.

Another aspect of the invention described herein provides a method for treating a spinal injury, comprising administering to a subject having a spinal injury an effective amount of CLP290.

In one embodiment of any aspect, CLP290 crosses the blood brain barrier. For example, CLP290 is formulated in a way that allows it to cross the blood brain barrier.

In one embodiment of any aspect, the subject is further administered at least a second spinal injury treatment. In one embodiment of any aspect, the subject is further administered at least a second therapeutic compound. In one embodiment of any aspect, the second therapeutic compound is selected from the group consisting of osteopontin, a growth factor, or 4-aminopuridine.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed technology, because the scope of the technology is limited only by the claims. Unless otherwise defined, all technical and scientific terms used herein have the same meaning as commonly understood by one of ordinary skill in the art to which this technology belongs. If there is an apparent discrepancy between the usage of a term in the art and its definition provided herein, the definition provided within the specification shall prevail.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a spinal cord injury. The term “treating” includes reducing or alleviating at least one adverse effect or symptom of a spinal cord injury, e.g., partial or complete paralysis. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of a spinal cord injury, delay or slowing of a spinal cord injury progression, amelioration or palliation of the injury state, remission (whether partial or total), and/or decreased mortality, whether detectable or undetectable. The term “treatment” of a spinal cord injury also includes providing relief from the symptoms or side-effects of the disease (including palliative treatment).

As used herein, the term “administering,” refers to the placement of a therapeutic (e.g., an agent that upmodulates KCC2 or reduces excitability of inhibitory interneurons) or pharmaceutical composition as disclosed herein into a subject by a method or route which results in at least partial delivery of the agent to the subject. Pharmaceutical compositions comprising agents as disclosed herein can be administered by any appropriate route which results in an effective treatment in the subject.

As used herein, a “subject” means a human or animal. Usually the animal is a vertebrate such as a primate, rodent, domestic animal or game animal. Primates include, for example, chimpanzees, cynomologous monkeys, spider monkeys, and macaques, e.g., Rhesus. Rodents include, for example, mice, rats, woodchucks, ferrets, rabbits and hamsters. Domestic and game animals include, for example, cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of spinal cord injury. A subject can be male or female.

A subject can be one who has been previously diagnosed with or identified as suffering from or having a spinal cord injury or one or more complications related to such an injury, and optionally, have already undergone treatment for a spinal cord injury or the one or more complications related to the injury. Alternatively, a subject can also be one who has not been previously diagnosed as having such spinal cord injury or related complications. For example, a subject can be one who exhibits one or more risk factors for a spinal cord injury, e.g., participates in an activity that is likely to result in a spinal cord injury, for example, a full contact sport, e.g., American football, or one or more complications related to spinal cord injury or a subject who does not exhibit risk factors.

Methods and compositions described herein are used for the treatment of a spinal cord injury. As used herein, a “spinal cord injury” refers to any insult to any region of the spinal cord, e.g., the cervical vertebrae, the thoracic vertebrae, the lumbar vertebrae, the sacral vertebrae, the sacrum, or the coccyx. A “spinal cord injury” can result in various levels of severity, ranging from no effect on mobility, e.g., retain walking ability, to paraplegia (e.g., paralysis of legs and lower region of body), and tretraplegia (e.g., loss of muscle strength in all four extremities). A “spinal cord injury” can be a complete spinal cord injury, e.g., an injury that produces total loss of all motor and sensory function below the site of injury. A “spinal cord injury” can be an incomplete spinal cord injury, e.g., in which some motor function remains below the primary site of the injury. Non-limiting examples of incomplete spinal cord injuries include, but are not limited to, anterior cord syndrome, center cord syndrome, and Brown-Sequard syndrome. A “spinal cord injury” can be a spinal concussion or spinal contusion, e.g., an injury that resolves itself in, e.g., one or two days. A spinal concussion or contusion can be complete or incomplete.

As used herein, an “agent” refers to e.g., a molecule, protein, peptide, antibody, or nucleic acid, that inhibits expression of a polypeptide or polynucleotide, or binds to, partially or totally blocks stimulation, decreases, prevents, delays activation, inactivates, desensitizes, or down regulates the activity of the polypeptide or the polynucleotide. Agents that inhibit NKCC, e.g., inhibit expression, e.g., translation, post-translational processing, stability, degradation, or nuclear or cytoplasmic localization of a polypeptide, or transcription, post transcriptional processing, stability or degradation of a polynucleotide or bind to, partially or totally block stimulation, DNA binding, transcription factor activity or enzymatic activity, decrease, prevent, delay activation, inactivate, desensitize, or down regulate the activity of a polypeptide or polynucleotide. An agent can act directly or indirectly.

The term “agent” as used herein means any compound or substance such as, but not limited to, a small molecule, nucleic acid, polypeptide, peptide, drug, ion, etc. An “agent” can be any chemical, entity or moiety, including without limitation synthetic and naturally-occurring proteinaceous and non-proteinaceous entities. In some embodiments, an agent is nucleic acid, nucleic acid analogues, proteins, antibodies, peptides, aptamers, oligomer of nucleic acids, amino acids, or carbohydrates including without limitation proteins, oligonucleotides, ribozymes, DNAzymes, glycoproteins, RNAis (e.g., microRNAs, siRNAs, and shRNAs) lipoproteins, aptamers, and modifications and combinations thereof etc. In certain embodiments, agents are small molecule having a chemical moiety. For example, chemical moieties included unsubstituted or substituted alkyl, aromatic, or heterocyclyl moieties including macrolides, leptomycins and related natural products or analogues thereof. Compounds can be known to have a desired activity and/or property, or can be selected from a library of diverse compounds.

The agent can be a molecule from one or more chemical classes, e.g., organic molecules, which may include organometallic molecules, inorganic molecules, genetic sequences, etc. Agents may also be fusion proteins from one or more proteins, chimeric proteins (for example domain switching or homologous recombination of functionally significant regions of related or different molecules), synthetic proteins or other protein variations including substitutions, deletions, insertion and other variants.

As used herein, the term “small molecule” refers to a chemical agent which can include, but is not limited to, a peptide, a peptidomimetic, an amino acid, an amino acid analog, a polynucleotide, a polynucleotide analog, an aptamer, a nucleotide, a nucleotide analog, an organic or inorganic compound (e.g., including heterorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

Methods and compositions described herein require that the level of KCC2 is upmodulated. As used herein, “K⁺—Cl⁻ co-transporter (KCC2)” refers to a protein with lower intracellular chloride concentrations below the electrochemical equilibrium potential. KCC2 can function in either a net efflux or influx pathway, depending on the chemical concentration gradients of potassium and chloride. Sequences for KCC2, also known as Solute carrier family 12 member 5, are known for a number of species, e.g., human KCC2 (NCBI Gene ID: 57468) polypeptide (e.g., NCBI Ref Seq NP_001128243.1) and mRNA (e.g., NCBI Ref Seq NM_001134771.1). KCC2 can refer to human KCC2, including naturally occurring variants, molecules, and alleles thereof. KCC2 refers to the mammalian KCC2 of, e.g., mouse, rat, rabbit, dog, cat, cow, horse, pig, and the like. The nucleic sequence of SEQ ID NO:1 comprises the nucleic sequence which encodes rat KCC2.

Methods and compositions described herein require that the levels and/or activity of NKCC are inhibited. As used herein, “Na+/2Cl−/K+ co-transporter (NKCC)” refers to a protein required to maintain proper ionic balance and cell volume by, e.g., mediating sodium and chloride transport and reabsorption. Sequences for NKCC, also known as Solute carrier family 12 member 2 and NKCC1, are known for a number of species, e.g., human NKCC (NCBI Gene ID: 6558) polypeptide (e.g., NCBI Ref Seq NP_001037.1) and mRNA (e.g., NCBI Ref Seq NM_001046.2). NKCC can refer to human NKCC, including naturally occurring variants, molecules, and alleles thereof. NKCC refers to the mammalian NKCC of, e.g., mouse, rat, rabbit, dog, cat, cow, horse, pig, and the like. The nucleic sequence of SEQ ID NO: 4 comprises the nucleic sequence which encodes NKCC.

Methods and compositions described herein require that the levels and/or activity of Kir2.1. are increased. As used herein, “Kir2.1” refers to potassium voltage-gated channel subfamily J member 2, characterized by having a greater tendency to allow potassium to flow into, rather than out of, a cell. Kir2.1 may participate in establishing action potential waveform and excitability of neuronal and muscle tissues. Kir2.1 sequences are known for a number of species, e.g., human Kir2.1 (NCBI Gene ID: 3759) polypeptide (e.g., NCBI Ref Seq NP_000882.1) and mRNA (e.g., NCBI Ref Seq NM_000891.2). Kir2.1 can refer to human Kir2.1, including naturally occurring variants, molecules, and alleles thereof. Kir2.1 refers to the mammalian Kir2.1 of, e.g., mouse, rat, rabbit, dog, cat, cow, horse, pig, and the like. The nucleic sequence of SEQ ID NO: 3 comprises an amino acid sequence which encodes human Kir2.1. The nucleic sequence of SEQ ID NO: 5 comprises an amino acid sequence which encodes mouse Kir2.1.

The term “upmodulation” and “upmodulate” as used herein refer to a change or an alteration that results in an increase in a biological activity (e.g., of KCC2, Gi-DREADD, or Kir2.1). Upmodulation includes, but is not limited to, stimulating or promoting an activity. Upmodulation may be a change in activity and/or levels, a change in binding characteristics, or any other change in the biological, functional, or immunological properties associated with the activity of a protein, a pathway, a system, or other biological targets of interest that results in its increased activity and/or levels. In some embodiments, the term “upmodulate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, a 20-fold increase, a 30-fold increase, a 40-fold increase, a 50-fold increase, a 60-fold increase, a 75-fold increase, a 100-fold increase, etc., or any increase between 2-fold and 10-fold or greater as compared to an appropriate control.

The term “decrease”, “reduced”, “reduction”, or “inhibit” are all used herein to mean a decrease by a statistically significant amount. In some embodiments, “decrease”, “reduced”, “reduction”, or “inhibit” typically means a decrease by at least 10% as compared to an appropriate control (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to an appropriate control.

The terms “increase”, “enhance”, or “activate” are all used herein to mean an increase by a reproducible statistically significant amount. In some embodiments, the terms “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, a 20 fold increase, a 30 fold increase, a 40 fold increase, a 50 fold increase, a 6 fold increase, a 75 fold increase, a 100 fold increase, etc. or any increase between 2-fold and 10-fold or greater as compared to an appropriate control. In the context of a marker, an “increase” is a reproducible statistically significant increase in such level.

As used herein, an “appropriate control” refers to an untreated, otherwise identical cell or population (e.g., a patient who was not administered an agent described herein, or was administered by only a subset of agents described herein, as compared to a non-control patient).

The term “pharmaceutically acceptable carrier” as used herein means a pharmaceutically acceptable material, composition or vehicle, such as a liquid or solid filler, diluent, excipient, solvent or encapsulating material, involved in carrying or transporting the active ingredient (e.g., cells) to the targeting place in the body of a subject. Each carrier must be “acceptable” in the sense of being compatible with the other ingredients of the formulation and is compatible with administration to a subject, for example a human.

The term “statistically significant” or “significantly” refers to statistical significance and generally means a two standard deviation (2SD) or greater difference.

As used herein the term “comprising” or “comprises” is used in reference to compositions, methods, and respective component(s) thereof, that are essential to the method or composition, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment. The term “consisting of” refers to compositions, methods, and respective components thereof as described herein, which are exclusive of any element not recited in that description of the embodiment.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

### BRIEF DESCRIPTION OF THE DRAWINGS

FIG. 1A-1K present data that show identification of CLP290 as a compound leading to functional recovery in mice with staggered lesions. (FIG. 1A) Schematic of staggered lateral hemisections at T7 and T10. Arrowheads indicate lesions, L=left, R=right. (FIG. 1B) Representative image of an anti-GFAP stained spinal cord section 10 weeks after over-stagger lesion. Dashed line indicates midline. Scale bar: 500 μm. (FIG. 1C) Representative image stacks of anti-5HT-stained transverse sections from T5 (rostral to lesions), T8 (between lesions), and L2 (caudal to lesions) of mice at 2 weeks after staggered lesions. Scale bar: 100 μm. (FIG. 1D) Experimental scheme. Each BMS test was performed 24 hr prior to daily compound treatment. (FIG. 1E) BMS scores in injured mice with continuous treatment of CLP290 (35 mg/kg) and vehicle solution. Two-way repeated-measures ANOVA followed by post hoc Bonferroni correction. Both groups started as n=10, and at week 9 (the termination time point) n=8, and 10 for vehicle and CLP290 respectively. *P<0.05; ****P<0.0001. Error bars: SEM. (FIG. 1F) Percentage of mice that reached stepping. CLP290 versus vehicle at 9 weeks post staggered injury (n=8 and 10 for vehicle and CLP290 group respectively). (FIG. 1G). Sustained behavioral improvements after CLP290 withdrawal in mice with 10-week treatment. BMS was tested on Day 1, 2, 3, 7 and 14 after compound withdrawal (n=7). Two-way repeated-measure ANOVA followed by post hoc Bonferroni correction. **p<0.01. Error bars: SEM. (FIG. 1H) Color-coded stick view decomposition of mouse right hindlimb movements during swing, stance (Intact group), dragging (Vehicle group) and stepping (CLP290 group). (FIG. 1I and FIG. 1J). Quantification of bodyweight support (FIG. 1I) and stride length (FIG. 1J) of mice at 9 weeks post staggered injury (n=8 and 10 for vehicle and CLP290 group respectively). Student's t-test (two-tailed, unpaired). *p<0.05; **p<0.01. Error bars: SEM. (FIG. 1K) Representative right hindlimb knee and ankle angle oscillation trace and simultaneous EMG recording from tibias anterior (TA) and gastrocnemius medialis (GS) muscle.

FIG. 2A-2H present data that show widespread KCC2 expression mimics the effects of CLP290 to promote functional recovery. (FIG. 2A) Experimental scheme. (FIG. 2B) Representative image stacks of longitudinal (upper) and transverse (lower) spinal cord sections, taken from the mice at 8 weeks after staggered injury, stained with anti-HA (to detect the HA-KCC2 protein). Scale bar: 500 μm (upper) and 100 μm (lower). (FIG. 2C) BMS performance in experimental (AAV-PHP.B-HA-KCC2) and control (AAV-PHP.B-H2B-GFP) groups. Two-way repeated-measures ANOVA followed by post hoc Bonferroni correction. *p<0.05. (FIG. 2D) Percentage of mice that reached stepping at 8 weeks after injury. (FIG. 2E and FIG. 2F) Quantification of bodyweight support (FIG. 2E) and stride length (FIG. 2F) at 8 weeks (n=10 per group). Student's t-test (two-tailed, unpaired) was applied. *p<0.05; **p<0.01. Error bars: SEM. (FIG. 2G) Color-coded stick view decomposition of mouse right hindlimb movement during dragging (AAV-PHP.B-H2B-GFP group) and stepping (AAV-PHP.B-HA-KCC2 group). (FIG. 2H) Representative right hindlimb knee and ankle angle oscillation trace and simultaneous EMG recording of mice at 8 weeks after injury.

FIG. 3A-3E present data that show KCC2 expression in inhibitory neurons leads to functional recovery. (FIG. 3A, 3B) Representative image stacks showing expression of GFP (FIG. 3A) or HA-KCC2 (FIG. 3B) in T8 spinal cord of indicated transgenic mice with tail-vein injection of AAV-PHP.B-CAG-Flex-H2B-GFP (FIG. 3A) or AAV-PHP.B-Syn-Flex-HA-KCC2 (FIG. 3B). Scale bar: 100 μm. (FIG. 3C) BMS performance in indicated groups. Two-way repeated-measure ANOVA followed by post hoc Bonferroni correction. *p<0.05; ****p<0.0001. Error bars: SEM. (FIG. 3D) Breakdown of BMS scores for indicated treatment groups at 8 weeks after injury. (FIG. 3E) Percentage of mice that reached plantar or dorsal stepping at 8 weeks after injury.

FIG. 4A-4H present data that show KCC2 acts on inhibitory neurons in the spinal cord segments between and around the lesions. (FIG. 4A) Experimental scheme for FIG. 4B-FIG. 4D. (FIG. 4B) Representative images of anti-HA-stained transverse sections of the thoracic and lumbar spinal cord at 8 weeks. Scale bar: 100 μm. (FIG. 4C and FIG. 4D) Left, BMS performance in different treatment groups in wild type mice (FIG. 4C), and Vgat-Cre mice (FIG. 4D). Right, percentage of mice that reached stepping in WT mice (FIG. 4C) and Vgat-Cre mice (FIG. 4D). ANOVA followed by post hoc Bonferroni correction. Error bars: SEM. (FIG. 4E) Experimental scheme for FIG. 4F-FIG. 4H. (FIG. 4F) Representative images of anti-HA-stained transverse sections of the thoracic and lumbar spinal cord at 8 weeks after injury. Scale bar: 100 μm. (FIG. 4G and FIG. 4H) Left, BMS performance in experimental and control groups in WT mice (FIG. 4G), and Vgat-Cre mice (FIG. 4H). Right, percentage of mice that reached stepping in WT mice (FIG. 4G) and Vgat-Cre mice (FIG. 4H). ANOVA followed by post hoc Bonferroni correction. *p<0.05. Error bars: SEM.

FIG. 5A-5F present data that show altered neuronal activation patterns and relay formation facilitated by CLP290/KCC2. (FIG. 5A) Schematics of transverse spinal cord sections showing c-Fos expression patterns in T8/9 segments after 1 hour of continuous locomotion in intact mice and injured mice with treatment of vehicle, CLP290, AAV-PHP.B-syn-HA-KCC2 or L838,417. Each spot represents a cell positively stained with both c-Fos and NeuN. Representative raw images are shown in FIG. 11A. (FIG. 5B) Average number of c-Fos+ neurons per section in the dorsal zone or the intermediate and ventral zones in all groups. One-way ANOVA followed by Bonferroni post hoc test (c-Fos+ NeuN+ numbers of the dorsal or intermediate/ventral zones in the Vehicle, CLP290, AAV-PHP.B-syn-HA-KCC2 or L838,417 treated groups were compared to that of the intact group, respectively). n=3 sections per mouse, n=3 mice per group. *p<0.05; ***P<0.001; ****P<0.0001; n.s. not significant. Error bars: SEM. (FIG. 5C) Average percentage of c-Fos+ neurons per section in Laminae 1-5 (Dorsal) or in Laminae 6-10 (Inter-ventral) in all groups One-way ANOVA followed by Bonferroni post hoc test (c-Fos+ NeuN+ percentages of the dorsal or intermediate/ventral zones in the Vehicle, CLP290, AAV-PHP.B-syn-HA-KCC2 or L838,417 treated groups were compared to that of the intact group, respectively). n=3 sections per mouse, n=3 mice per group, *p<0.05; **P<0.01; ***P<0.001; n.s. not significant. Error bars: SEM. (FIG. 5D) Left, schematic of cortical stimulation and TA muscle EMG experiments. Right, representative responses in the right TA muscle evoked by a train of epidural motor cortex stimulations in STA control, AAV-PHP.B-syn-HA-KCC2, CLP290 treated, full transection, and intact groups. (FIG. 5E) Right TA muscle EMG response amplitude from indicated groups. One-way ANOVA followed by Bonferroni post hoc test. n=3 attempts per mouse, n=3 mice per group, ***p<0.001; n.s. not significant; error bars, SEM. (FIG. 5F) Right TA muscle EMG response latency from indicated groups. One-way ANOVA followed by Bonferroni post hoc test. n=3 attempts per mouse, n=3 mice per group, ***p<0.001; n.s. not significant. Error bars: SEM.

FIG. 6A-6F present data that show Gi-DREADD expression in inhibitory interneurons between and around the lesion mimics the effects of KCC2/CLP290. (FIG. 6A) Experimental scheme. (FIG. 6B) Representative images of transverse sections of the thoracic and lumbar spinal cord at 8 weeks post-SCI immunostained with anti-RFP to indicate hM4Di DREADD expression. Scale bar: 100 μm. (FIG. 6C) BMS performance over time after SCI and virus injections in Gi-DREADD and GFP groups in Vgat-Cre mice. ANOVA followed by post hoc Bonferroni correction. **p<0.001, ****p<0.0001, error bars, SEM. (FIG. 6D). Schematic of transverse spinal cord sections showing c-Fos positive neurons in T8/9 segments after 1 hour of continuous locomotion in AAV-9-Syn-Gi-DREADD treated mice (dorsal/plantar stepping) and AAV-9-Syn-GFP mice group (dragging). (FIG. 6E) Average numbers of c-Fos+ neurons (all laminae) per section in indicated groups. Student's t-test (two-tailed, unpaired). n=3 sections per mouse, n=3 mice per group. n.s. not significant. Error bars: SEM. (FIG. 6F) Percentage of c-Fos+ neurons in Laminae 1-5 or Laminae 6-10 in indicated groups. Student's t-test (two-tailed, unpaired). n=9 sample slides per group, n=3 mice per group. **P<0.01; n.s. not significant. Error bars: SEM.

FIG. 7A-7F present data that show effects of small molecule compounds in mice with staggered or complete spinal cord injury. (FIG. 7A) BMS scores measured at 24 hr after compound administration in stagger-lesioned mice with continuous treatment of indicated compounds. Repeated measures ANOVA followed by post hoc Bonferroni correction. All groups started as n=10, and at week 9 (the termination time point) n=8, 10, 3, 8, 4, 7 and 7 for saline, CP101606 (10 mg/Kg), bumetanide (0.3 mg/Kg), baclofen (1 mg/Kg), L838,417 (1 mg/Kg), 8-OH-DPAT (0.1 mg/Kg) and quipazine (0.2 mg/Kg) respectively. Error bars, SEM. (FIG. 7B) BMS scores measured acutely after compound treatments (10, 30, 30, 60 and 120 min after compound administration) in stagger-lesioned mice at 8 weeks after SCI. Two way repeated measures ANOVA followed by post hoc Bonferroni correction. All groups n=5, ****P<0.0001; error bars, SEM. (FIG. 7C) Representative confocal images of transverse sections, stained with anti-5HT antibody, from L2 spinal level of injured mice with CLP290 treatment at 10 weeks post staggered injury. Scale bar: 100 μm. (FIG. 7D) Left, Schematic of full transection (FT) at T8. Arrowhead indicates lesion. Right top: Representative confocal image stack of a longitudinal spinal cord section (from T5 to T12) at 10 weeks post FT lesion immunostained with anti-GFAP. Dashed line indicates midline. Scale bar: 500 Right bottom: Representative confocal image stacks of transverse sections from the thoracic and lumbar spinal cord (T5, rostral to lesions, T9 and L2, caudal to lesion) at 8 weeks post over-stagger lesion immunostained with anti-5HT (serotonergic axons). Scale bar: 100 μm. (FIG. 7E) BMS scores measured at 24 hr after vehicle or CLP290 administration in mice with full transection. Repeated measures ANOVA followed by post hoc Bonferroni correction. Both groups started as n=10, and at week 9 (the termination time point) n=8, and 10 for vehicle and CLP290 respectively. Error bars, SEM. (FIG. 7F) BMS scores measured acutely after compound treatments (10, 30, 30, 60 and 120 min after compound administration) at 8 weeks in mice after full transection without chronic treatments. Repeated measures ANOVA followed by post hoc Bonferroni correction. All groups n=5, ****P<0.0001; error bars, SEM.

FIG. 8A-8F present data that show no significant effects of CLP290 on axon growth (Retrograde labeling). (FIG. 8A) Left: Schematic of HiRet-mCherry injection to retrogradely labeled propriospinal and brain neurons with descending projections to right side lumbar spinal cord (L2-4). Mice received HiRet-mCherry injection at either 1 day (acute) or 8 weeks (chronic) after injury. The mice were terminated at 2 weeks after viral injection for histological analysis. Middle: Longitudinal representations of propriospinal neurons labeled at acute and chronic stages. Each dot represents 5 neurons. Right: Representative confocal image stacks of transverse sections of T8 (between the lesions) and T13 (below the lesions) at 10 weeks post staggered injury stained with anti-RFP. Scale bar: 100 Bottom: Ipsi-tracing PNs: ipsilateral tracing propriospinal neurons, Midline-crossing PNs: middle line crossing propriospinal neurons (relative to injection site). (FIG. 8B and FIG. 8C) Quantification of labeled neurons in the brain and spinal cord from A. Numbers of retrogradely labeled neurons in different brain regions and spinal segments in mice with vehicle treatment at acute and chronic stages (FIG. 8B) or in mice with vehicle or CLP290 treatment at chronic stage (FIG. 8C) were normalized to those retrogradely labeled neurons in intact mice Rostral: above T7; inter, T8-T10; caudal: T10-L1. L: left, R: right. Student's t test; n=3 each for intact, acute and chronic SCI mice. *P<0.05, n.s. not significant. Error bars: SEM. (FIG. 8D) Left: Schematic of HiRet-mCherry injection to retrogradely label propriospinal and brain neurons with descending projections to left side lumbar spinal cord (L2-4). Animals received HiRet-mCherry injection at either 1 day (acute) or 8 weeks (chronic) after staggered injury. The mice were terminated at 2 weeks after viral injection for histological analysis. Middle: Longitudinal representations of propriospinal neurons labeled at acute and chronic stages. Each dot represents 5 neurons. Right: Representative confocal image stacks of transverse sections of T8 (between the lesions) and T13 (below the lesions) at 10 weeks post staggered injury stained with anti-RFP. Scale bar: 100 Bottom: Ipsi-tracing PNs: ipsilateral tracing propriospinal neurons, Midline-crossing PNs: middle line crossing propriospinal neurons (relative to injection site). (FIG. 8E and FIG. 8F) Quantification of labeled neurons in the brain and spinal cord from D. Numbers of mCherry-marked of brain and propriospinal neurons in different spinal segments in mice with vehicle treatment at acute and chronic stages (FIG. 8E) or in mice with vehicle or CLP290 treatment at chronic stage (FIG. 8F) were normalized to those retrogradely labeled neurons in intact mice. Rostral: above T7; inter, T8-T10; caudal: T10-L1. L: left, R: right. Student's t test; n=3 each for intact, acute and chronic SCI mice. * P<0.05, n.s. not significant. Error bars: SEM.

FIG. 9A-9I present data that show no effects of CLP290 on axon growth of descending axons. (FIG. 9A) Left: Schematic of AAV injection strategy for anterograde labeling of neurons from brainstem reticular formation. Animals received an injection of AAV-ChR2-mCherry (left) and AAV-ChR2-GFP (right side) at either 1 day (acute) or 8 weeks (chronic) after injury. The mice were terminated at 2 weeks after viral injection for histological analysis. Black line: axons descending from left side reticular formation; gray line: axons descending from right side reticular formation. Right: Representative confocal image stacks of transverse sections of the thoracic and lumbar spinal cord at 2 weeks and 10 weeks post injury stained with anti-RFP and anti-GFP. Scale bar: 100 (FIG. 9B) The fluorescence intensity of mCherry and GFP immunostaining at 2 weeks and 10 weeks post staggered injury in vehicle treated groups. All images were acquired using identical imaging parameters and scan settings. In each case, the intensities were normalized to 2 weeks post staggered injury in the rostral level. Student's t test; n=3 sections per mouse and n=3 mice per group. *p<0.05 and ns, not significant. Error bar: SEM. (FIG. 9C) The fluorescence intensity of mCherry and GFP immunostaining at 10 weeks post staggered injury in the vehicle treated and CLP290 treated groups. All images were acquired using identical imaging parameters and scan settings. In each case, the intensities were normalized to 2 weeks post staggered injury in rostral levels. Student's t test; n=3 sections per mouse and n=3 mice per group. *p<0.05 and ns, not significant. Error bar: SEM. (FIG. 9D) Schematic and images to show serotonergic axons in different levels of the spinal cord taken from 2 or 10 weeks after injury with or without CLP290 treatment. (FIG. 9E, FIG. 9F). The fluorescence intensity of 5-HT immunostaining was compared at acute and chronic stages for vehicle treated groups (FIG. 9E), and also compared at chronic stages between vehicle and CLP290 treated groups (FIG. 9F). Student's t test; n=3 sections per mouse and n=3 mice per group. *p<0.05 and ns, not significant. Error bar: SEM. (FIG. 9G-FIG. 9I). (FIG. 9G) AAV-ChR2-GFP injected to the right cortex to trace CST axon terminations in different spinal cord levels in 2 or 10 week after injury with or without CLP290 treatment. The fluorescence intensity of anti-GFP immunostaining was compared between acute and chronic stages in vehicle treated mice (FIG. 9H), and between vehicle or CLP290 treated groups at 10 weeks after injury (FIG. 9I). Scale bar: 100 Student's t test; n=3 sections per mouse and n=3 mice per group. ns, not significant. Error bar: SEM.

FIG. 10A-10E present data that show AAV-mediated KCC2 expression in spinal neurons and its behavioral outcomes. (FIG. 10A, FIG. 10B) Representative Western blotting images and quantification showing KCC2 protein levels in the inter-lesion region (T8/9) (FIG. 10A) and in the lumbar spinal cord (L2-4) (FIG. 10B) of intact or stagger lesioned mice treated with either AAV-PHP.B-FLEX-GFP or AAV-PHP.B-HA-KCC2, at 10 weeks after injury. Actin as a loading control. n=6, 5 and 5 mice for intact, AAV-PHP.B-GFP and AAV-PHP.B-HA-KCC2 group respectively. Student's t test; *P<0.05; **P<0.01; Error bars: SEM. (FIG. 10C) Left, Schematic of experimental design. AAV virus was intraspinally injected into lumbar segments (L2-4) of experimental (AAV-1-Syn-HA-KCC2) and control mice (AAV-1-Syn-GFP-H2B). Right, representative confocal image stack of a longitudinal spinal cord section (from T5 to 51) at 10 weeks post staggered injury immunostained with anti-HA to label virally expressed KCC2. (FIG. 10D) Left, Schematic of experimental design. AAV virus was injected into the tail vein of experimental (AAV-9-Syn-HA-KCC2) and control (AAV-9-Syn-GFP-H2B) mice. Right, representative confocal image stack of a longitudinal spinal cord section (from T5 to L3) at 10 weeks post staggered injury immunostained with anti-HA to label virally expressed KCC2. Scale bar: 500 μm. (FIG. 10E) BMS scores measured at 24 hr in Vgat-Cre mice with tail vein injection of AAV-9-Syn-HA-KCC2 and treatment of vehicle or CLP290. Both groups started as n=8, and at week 9 (the termination time point) n=6 for both vehicle and CLP290 respectively. Repeated measures ANOVA followed by post hoc Bonferroni correction. **P<0.01; error bars, SEM.

FIG. 11A-11D present data that show altered c-Fos expression patterns in T8/9 of stagger-lesioned mice with different treatments. (FIG. 11A) Representative confocal image stacks of transverse sections from T8/9 spinal cord at 8 weeks after injury stained with antibody against c-Fos, NeuN or both c-Fos and NeuN. Scale bar: 100 μm. (FIG. 11B) Percentages of NeuN+ cells among c-fos+ cells in intact mice or injured mice with individual treatments (vehicle control, CLP290, AAV-PHP.B-HA-KCC2 and L838,417). One-way ANOVA followed by Bonferroni post hoc test. n=3 sections per mouse, n=3 mice per group. n.s. not significant. Error bars: SEM. (FIG. 11C) Average number of c-Fos+ neurons per section in dorsal zone or in intermediate and ventral zones of staggered-lesioned mice with the treatment of vehicle (STA), continuous CLP290 treatment (CLP290), and 2 weeks after CLP290 withdrawal (CLP290 withdrawal). One-way ANOVA followed by Bonferroni post hoc test (c-Fos+ NeuN+ numbers of the dorsal or intermediate/ventral zones in the CLP290, or CLP290 withdrawal groups were compared to that of the vehicle group, respectively). n=3 sections per mouse, n=3 mice per group. *p<0.05; **P<0.01; n.s. not significant. Error bars: SEM. (FIG. 11D) Average percentage of c-Fos+ neurons per section in Laminae 1-5 or in Laminae 6-10 in staggered-lesioned mice with the treatment of vehicle (STA), continuous CLP290 treatment (CLP290), and 2 weeks after CLP290 withdrawal (CLP290 withdrawal). One-way ANOVA followed by Bonferroni post hoc test (c-Fos+ NeuN+ percentages of the dorsal or intermediate/ventral zones in the CLP290, or CLP290 withdrawal groups were compared to that of the vehicle group, respectively). n=3 sections per mouse, n=3 mice per group, **P<0.01; n.s. not significant. Error bars: SEM.

FIG. 12A-12C present data that show Gq-DREADD expression. (FIG. 12A) Representative confocal images of transverse sections of the thoracic and lumbar spinal cord at 8 weeks post staggered injury stained with anti-RFP to indicate hM3D DREADD expression. Scale bar: 100 μm. (FIG. 12B) BMS scores of staggered injured Vglut2-Cre mice with viral injection of AAV9-Syn-FLEX-GFP or AAV9-FLEX-hM3Dq-mCherry. Repeated measures ANOVA followed by post hoc Bonferroni correction. n=5 for each group. Error bars: SEM. (FIG. 12C) BMS scores measured acutely after compound treatments (10, 30, 60, 120 and 180 min after CNO administration) in stagger-lesioned vGlut2-Cre mice at 8 weeks after SCI. Repeated measures ANOVA followed by post hoc Bonferroni correction. n=5, *P<0.05; ***P <0.001; error bars, SEM.

FIG. 13A-13C present data that show efficacy of treatment with AAV-PHP.B-HA-KCC2 in spinal cord injury model. (FIG. 13A) BMS scores in T10 contusion injured mice with KCC2 treatment (AAV-PHP.B-HA-KCC2) and control. Two-way repeated-measures ANOVA followed by post hoc Bonferroni correction. * P<0.05, **P<0.01. Error bars, SEM. (n=11 in control group, n=10 in KCC2 group). (FIG. 13B) Quantification of bodyweight support (top) and step height (bottom) 8 weeks after contusion injury (n=11 in control group, n=10 in KCC2 group). Student's t test (two-tailed, unpaired) was applied. *p<0.05; **p<0.01. Error bars, SEM. (FIG. 13C) Percentage of mice that reached stepping at 8 weeks after injury (top). Percentage of mice that had spasticity at 8 weeks after injury (bottom). Injured mice were classified as “spasticity-strong” if they showed spasm over 50% BMS scoring time (n=11 in control group, n=10 in KCC2 group).

### DETAILED DESCRIPTION

The invention described herein is based, in part, on the discovery that a KCC2 agonist restored stepping ability in mice with staggered bilateral hemisections, e.g., an injury in which the lumbar spinal cord is deprived of all direct brain-derived innervation but dormant relay circuits remain. It was further found that this restoration of stepping ability can additionally be mimicked by selective expression of KCC2, or hyperpolarizing DREADDs (e.g., optimized Gi-DREADD) in the inhibitory interneurons between and around the staggered spinal lesions.

Additionally, provided herein is evidence that shows the inhibition or NKCC, or the expression of Kir2.1 results in the increased stepping ability in mice who have previously lost this ability due to, e.g., a staggered bilateral hemisection. Mechanistically, these treatments transformed this injury-induced dysfunctional spinal circuit to a functional state, facilitating the relay of brain-derived commands towards the lumbar spinal cord.

Thus, provided herein are methods for increasing expression of KCC2, Gi-DREADD, or Kir2.1, or inhibiting NKCC, in patients having a spinal cord injury. Additionally, described herein are compositions comprising agents useful for increasing expression of KCC2, Gi-DREADD, or Kir2.1, or inhibiting NKCC. Further provided herein are compositions comprising agents that modulate KCC2, NKCC, Gi-DREAD, or Kir2.1 for the use of treatment of a spinal cord injury

Treating a Spinal Cord Injury

Methods provided herein are directed at treating a spinal cord injury. In one embodiment, the spinal injury is a severe spinal injury. A spinal cord injury refers to any insult to the any region of the spinal cord, e.g., the cervical vertebrae, the thoracic vertebrae, the lumbar vertebrae, the sacral vertebrae, the sacrum, or the coccyx, that causes a negative effect on the function of the spinal cord, e.g., reduce mobility of feeling in limbs. A severity of a spinal cord injury is measured in levels of the injury's outcome, e.g., ranging from no effect on mobility, e.g., retained walking capacity, to paraplegia (e.g., paralysis of legs and lower region of body), and tretraplegia (e.g., loss of muscle strength in all four extremities). In one embodiment, the methods and compositions described herein are used to treat a severe spinal cord injury. As used herein, “severe spinal cord injury” refers to the complete or incomplete spinal cord injury that produces total loss of all motor and sensory function below the level of injury.

One aspect of the invention provides a method for treating a spinal injury, comprising administering to a subject having a spinal injury an effective amount of an agent that upmodulates neuron-specific K⁺—Cl⁻ co-transporter (KCC2).

A second aspect of the invention provides a method for treating a spinal injury, comprising administering to a subject having a spinal injury an effective amount of an agent that inhibits Na+/2Cl−/K+ co-transporter (NKCC).

A third aspect of the invention provides a method for treating a spinal injury, comprising administering to a subject having a spinal injury an effective amount of an agent that reduces excitability of inhibitory interneurons. In one embodiment, the agent upmodulates the inhibitory Gi-coupled receptor Gi-DREADD. Gi-coupled DREADD refers to a designer receptor exclusively activated by designer drugs (DREADD). Gi-DREADD can be expressed in a specific localization, e.g., expressed on inhibitory interneurons, and can be controlled, e.g., via its agonist or antagonist. DREADDs are further described in, e.g., Saloman, J L, et al. Journal of neuroscience. 19 Oct. 2016: 36 (42); 10769-10781, which is incorporated herein by reference in its entirety.

Used herein is a Gi-DREADD optimized for expression in the inhibitory interneurons. In one embodiment, Gi-DREADD is expressed in the spinal cord. In one embodiment, Gi-DREADD is expressed at the site of injury. In one embodiment, Gi-DREADD is expressed on inhibitory interneurons. In yet another embodiment, the agent is administered at substantially the same time as an agonist of Gi-DREADD, e.g., clozapine N-oxide. In another embodiment, the agent upmodulates Kir2.1.

A fourth aspect of the invention provides a method for treating a spinal injury, comprising administering to a subject having a spinal injury an effective amount electrical stimulation that reduces excitability of inhibitory interneurons. Electrostimulation, also known as epidural spinal electrostimulation, is a method in the treatment for subjects suffering from chronic pain or severe central motor disturbance, e.g., due to a spinal cord injury. Electrostimulation is the application of a continuous electrical current to the lower part of the spinal cord, e.g., via a chip implanted over the dura (e.g., the protective coating) of the spinal cord. The chip is controlled, e.g., via a remote to vary the frequency and intensity of the electrical current. In one embodiment, electrostimulation is applied directly to the spinal cord, but not at the site of injury (e.g., on an uninjured part of the spinal cord). In another embodiment, electrostimulation is applied directly to the spinal cord at the site of injury. In one embodiment, the method further comprises administering an agonist of Gi-DREADD, e.g., clozapine N-oxide.

In one embodiment, electrostimulation as described herein reduces the excitability of inhibitory interneurons is reduced by at least 10%, at least 20%, at least 30%, at least 40%, at least 50%, at least 60%, at least 70%, at least 80%, at least 90, at least 99%, or more as compared to an appropriate control. As used in this context, an appropriate control refers to the excitability of an unstimulated inhibitory intereneuron.

In one embodiment of various aspects, prior to administration, the subject is diagnosed with a spinal cord injury. A skilled clinician can diagnose a subject as having a spinal cord injury via, e.g., a physical exam, or a radiological diagnostic approach, such as an X-ray, a computerized tomography (CT) scan, and/or a magnetic resonance imaging (MM) scan.

In various embodiments, the subject can have previously been diagnosed with having a spinal cord injury, and can have previously been treated for a spinal cord injury.

Agents

Described herein are agents that upmodulate KCC2. In one embodiment, the agent that upmodulates KCC2 is a small molecule, a peptide, a gene editing system, or an expression vector encoding KCC2. In one embodiment, the small molecule that upmodulates KCC2 is CLP290, or a derivative thereof. An agent is considered effective for upmodulates KCC2 if, for example, upon administration, it increases the presence, amount, activity and/or level of KCC2 in the cell. In one embodiment, KCC2 is upmodulated by at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, a 20-fold increase, a 30-fold increase, a 40-fold increase, a 50 fold increase, a 60-fold increase, a 75-fold increase, a 100-fold increase, etc. or any increase between 2-fold and 10-fold or greater as compared to an appropriate control. As used herein in this context, an appropriate control refers to the levels of KCC2 in an untreated cell. A skilled person can measure the levels of KCC2 using techniques described herein, e.g., western blotting or PCR-based assays to assess KCC2 protein or mRNA levels, respectively.

CLP290 is a small molecule enhancer of KCC2 activity. CLP290 is also known in the art as [5-Fluoro-2-[(Z)-(2-hexahydropyridazin-1-yl-4-oxo-thiazol-5-ylidene)methyl]phenyl] pyrrolidine-1-carboxylate, and has a structure of:

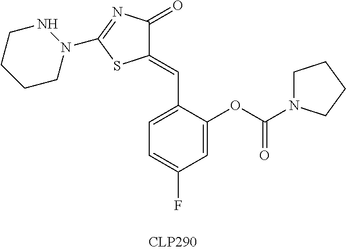 [SMILES: O=C1N=C(N2CCCCN2)S/C1=C\c1ccc(F)cc1OC(=O)N1CCCC1]

Further, in one embodiment, the small molecule is a derivative, a variant, or an analog of any of the small molecules described herein, for example CLP290. A molecule is said to be a “derivative” of another molecule when it contains additional chemical moieties not normally a part of the molecule and/or when it has been chemically modified. Such moieties can improve the molecule's expression levels, enzymatic activity, solubility, absorption, biological half-life, etc. The moieties can alternatively decrease the toxicity of the molecule, eliminate or attenuate any undesirable side effect of the molecule, etc. Moieties capable of mediating such effects are disclosed in Remington's Pharmaceutical Sciences, 18th edition, A. R. Gennaro, Ed., Mack Publ., Easton, Pa. (1990). A “variant” of a molecule is meant to refer to a molecule substantially similar in structure and function to either the entire molecule, or to a fragment thereof. A molecule is said to be “substantially similar” to another molecule if both molecules have substantially similar structures and/or if both molecules possess a similar biological activity. Thus, provided that two molecules possess a similar activity, they are considered variants as that term is used herein even if the structure of one of the molecules not found in the other, or if the structure is not identical. An “analog” of a molecule is meant to refer to a molecule substantially similar in function to either the entire molecule or to a fragment thereof.

Also described herein are agents that inhibit NKCC. In one embodiment, the agent that inhibits NKCC is a small molecule, an antibody, a peptide, an antisense oligonucleotide, or an RNAi. In one embodiment, the small molecule that upmodulates KCC2 is bumetanide, or a derivative thereof. An agent is considered effective for inhibiting NKCC if, for example, upon administration, it inhibits the presence, amount, activity and/or level of NKCC in the cell. In one embodiment, NKCC is inhibited at least 10%, at least 20%, at least 30%, at least 40%, at least 50%, at least 60%, at least 70%, at least 80%, at least 90, at least 99%, or more as compared to an appropriate control. As used herein in this context, an appropriate control refers to the level of NKCC in an untreated cell. A skilled person can measure the levels of NKCC using techniques described herein, e.g., western blotting or PCR-based assays to assess NKCC protein or mRNA levels, respectively.

Additionally, described herein is an expression vector encoding Gi-DREADD for expression of Gi-DREADD in inhibitory interneurons to reduce the excitability of inhibitory interneurons. The expression vector is considered effective for expressing Gi-DREADD if, for example, upon administration, it increases the presence, amount, activity and/or level of Gi-DREADD in the cell. In one embodiment, expression of Gi-DREADD reduces the excitability of inhibitory intereneurons by at least 10%, at least 20%, at least 30%, at least 40%, at least 50%, at least 60%, at least 70%, at least 80%, at least 90, at least 99%, or more as compared to an appropriate control. As used herein in this context, an appropriate control refers to an otherwise identical population of untreated inhibitory interneurons. A skilled person can measure the levels of Gi-DREADD using techniques described herein, e.g., western blotting or PCR-based assays to assess Gi-DREADD protein or mRNA levels, respectively. A skilled person can measure the excitability of inhibitor interneurons, e.g., by measuring c-fos levels which is expressed in the nucleus of an excitatory and inhibitory interneuron, e.g., via immunostaining a biological sample, or electrophysiological recordings (e.g., a direct measurement of the electrical activity of a neuron, for example, an inhibitory interneuron). A reduction in c-Fos levels would indicate reduced excitably in the inhibitory interneurons has been achieved. Methods for performing electrophysiological recordings, e.g., in the neurons, is further reviewed in, e.g., Du C., et al. ASC Biomater. Sci. Eng. 2017, 3(10), pp 2235-2246, which is incorporated herein by reference in its entirety.

Additionally, described herein is an expression vector encoding Kir2.1 for expression of Kir2.1 in inhibitory interneurons to reduce the excitability of inhibitory interneurons. The expression vector is considered effective for expressing Kir2.1 if, for example, upon administration, it increases the presence, amount, activity and/or level of Kir2.1 in the cell. In one embodiment, expression of Kir2.1 reduces the excitability of inhibitory intereneurons by at least 10%, at least 20%, at least 30%, at least 40%, at least 50%, at least 60%, at least 70%, at least 80%, at least 90, at least 99%, or more as compared to an appropriate control. As used herein in this context, an appropriate control refers to an otherwise identical population of untreated inhibitory interneurons. A skilled person can measure the levels of Kir2.1 using techniques described herein, e.g., western blotting or PCR-based assays to assess Kir2.1 protein or mRNA levels, respectively. A skilled person can measure the excitability of inhibitor interneurons as described herein above.

An agent can inhibit, e.g., the transcription or the translation of NKCC in the cell. An agent can inhibit the activity or alter the activity (e.g., such that the activity no longer occurs, or occurs at a reduced rate) of NKCC in the cell (e.g., NKCC's expression).

An agent can increase e.g., the transcription, or the translation of, e.g., KCC2, Gi-DREADD, or Kir2.1 in the cell. An agent can increase the activity or alter the activity (e.g., such that the activity occurs more frequently, or occurs at an increased rate) of, e.g., KCC2, Gi-DREADD, or Kir2.1 in the cell (e.g., KCC2, Gi-DREADD, or Kir2.1's expression).

The agent may function directly in the form in which it is administered. Alternatively, the agent can be modified or utilized intracellularly to produce something which, e.g., upmodulates KCC2, Gi-DREADD, or Kir2.1, or inhibits NKCC, such as introduction of a nucleic acid sequence into the cell and its transcription resulting in the production, for example of the nucleic acid and/or protein inhibitor of NKCC, or nucleic acid and/or protein that upmodulates KCC2, Gi-DREADD, or Kir2.1 within the cell. In some embodiments, the agent is any chemical, entity or moiety, including without limitation synthetic and naturally-occurring non-proteinaceous entities. In certain embodiments the agent is a small molecule having a chemical moiety. For example, chemical moieties included unsubstituted or substituted alkyl, aromatic, or heterocyclyl moieties including macrolides, leptomycins and related natural products or analogues thereof. Agents can be known to have a desired activity and/or property, or can be identified from a library of diverse compounds.

In various embodiments, the agent is a small molecule that upmodulates KCC2, or inhibits NKCC. Methods for screening small molecules are known in the art and can be used to identify a small molecule that is efficient at, for example, inducing cell death of pathogenic CD4 cells, given the desired target (e.g., KCC2, or NKCC).

In various embodiments, the agent that inhibits NKCC is an antibody or antigen-binding fragment thereof, or an antibody reagent that is specific for NKCC. As used herein, the term “antibody reagent” refers to a polypeptide that includes at least one immunoglobulin variable domain or immunoglobulin variable domain sequence and which specifically binds a given antigen. An antibody reagent can comprise an antibody or a polypeptide comprising an antigen-binding domain of an antibody. In some embodiments of any of the aspects, an antibody reagent can comprise a monoclonal antibody or a polypeptide comprising an antigen-binding domain of a monoclonal antibody. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody reagent” encompasses antigen-binding fragments of antibodies (e.g., single chain antibodies, Fab and sFab fragments, F(ab′)2, Fd fragments, Fv fragments, scFv, CDRs, and domain antibody (dAb) fragments (see, e.g. de Wildt et al., Eur J. Immunol. 1996; 26(3):629-39; which is incorporated by reference herein in its entirety)) as well as complete antibodies. An antibody can have the structural features of IgA, IgG, IgE, IgD, or IgM (as well as subtypes and combinations thereof). Antibodies can be from any source, including mouse, rabbit, pig, rat, and primate (human and non-human primate) and primatized antibodies. Antibodies also include midibodies, nanobodies, humanized antibodies, chimeric antibodies, and the like.

NKCC is an antisense oligonucleotide. As used herein, an “antisense oligonucleotide” refers to a synthesized nucleic acid sequence that is complementary to a DNA or mRNA sequence, such as that of a microRNA. Antisense oligonucleotides are typically designed to block expression of a DNA or RNA target by binding to the target and halting expression at the level of transcription, translation, or splicing. Antisense oligonucleotides of the present invention are complementary nucleic acid sequences designed to hybridize under cellular conditions to a gene, e.g., NKCC. Thus, oligonucleotides are chosen that are sufficiently complementary to the target, i.e., that hybridize sufficiently well and with sufficient specificity in the context of the cellular environment, to give the desired effect. For example, an antisense oligonucleotide that inhibits NKCC may comprise at least 5, at least 10, at least 15, at least 20, at least 25, at least 30, or more bases complementary to a portion of the coding sequence of the human NKCC gene (e.g., SEQ ID NO: 4), respectively.

SEQ ID NO: 4 is a nucleic acid sequence encoding NKCC.

(SEQ ID NO: 4)
atggag ccgcggccca
cggcgccctc ctccggcgcc ccgggactgg ccggggtcgg ggagacgccg tcagccgctg
cgctggccgc agccagggtg gaactgcccg gcacggctgt gccctcggtg ccggaggatg
ctgcgcccgc gagccgggac ggcggcgggg tccgcgatga gggccccgcg gcggccgggg
acgggctggg cagacccttg gggcccaccc cgagccagag ccgtttccag gtggacctgg
tttccgagaa cgccgggcgg gccgctgctg cggcggcggc ggcggcggcg gcagcggcgg
cggctggtgc tggggcgggg gccaagcaga cccccgcgga cggggaagcc agcggcgaga
gcgagccggc taaaggcagc gaggaagcca agggccgctt ccgcgtgaac ttcgtggacc
cagctgcctc ctcgtcggct gaagacagcc tgtcagatgc tgccggggtc ggagtcgacg
ggcccaacgt gagcttccag aacggcgggg acacggtgct gagcgagggc agcagcctgc
actccggcgg cggcggcggc agtgggcacc accagcacta ctattatgat acccacacca
acacctacta cctgcgcacc ttcggccaca acaccatgga cgctgtgccc aggatcgatc
actaccggca cacagccgcg cagctgggcg agaagctgct ccggcctagc ctggcggagc
tccacgacga gctggaaaag gaaccttttg aggatggctt tgcaaatggg gaagaaagta
ctccaaccag agatgctgtg gtcacgtata ctgcagaaag taaaggagtc gtgaagtttg
gctggatcaa gggtgtatta gtacgttgta tgttaaacat ttggggtgtg atgcttttca
ttagattgtc atggattgtg ggtcaagctg gaataggtct atcagtcctt gtaataatga
tggccactgt tgtgacaact atcacaggat tgtctacttc agcaatagca actaatggat
ttgtaagagg aggaggagca tattatttaa tatctagaag tctagggcca gaatttggtg
gtgcaattgg tctaatcttc gcctttgcca acgctgttgc agttgctatg tatgtggttg
gatttgcaga aaccgtggtg gagttgctta aggaacattc catacttatg atagatgaaa
tcaatgatat ccgaattatt ggagccatta cagtcgtgat tcttttaggt atctcagtag
ctggaatgga gtgggaagca aaagctcaga ttgttctttt ggtgatccta cttcttgcta
ttggtgattt cgtcatagga acatttatcc cactggagag caagaagcca aaagggtttt
ttggttataa atctgaaata tttaatgaga actttgggcc cgattttcga gaggaagaga
ctttcttttc tgtatttgcc atcttttttc ctgctgcaac tggtattctg gctggagcaa
atatctcagg tgatcttgca gatcctcagt cagccatacc caaaggaaca ctcctagcca
ttttaattac tacattggtt tacgtaggaa ttgcagtatc tgtaggttct tgtgttgttc
gagatgccac tggaaacgtt aatgacacta tcgtaacaga gctaacaaac tgtacttctg
cagcctgcaa attaaacttt gatttttcat cttgtgaaag cagtccttgt tcctatggcc
taatgaacaa cttccaggta atgagtatgg tgtcaggatt tacaccacta atttctgcag
gtatattttc agccactctt tcttcagcat tagcatccct agtgagtgct cccaaaatat
ttcaggctct atgtaaggac aacatctacc cagctttcca gatgtttgct aaaggttatg
ggaaaaataa tgaacctctt cgtggctaca tcttaacatt cttaattgca cttggattca
tcttaattgc tgaactgaat gttattgcac caattatctc aaacttcttc cttgcatcat
atgcattgat caatttttca gtattccatg catcacttgc aaaatctcca ggatggcgtc
ctgcattcaa atactacaac atgtggatat cacttcttgg agcaattctt tgttgcatag
taatgttcgt cattaactgg tgggctgcat tgctaacata tgtgatagtc cttgggctgt
atatttatgt tacctacaaa aaaccagatg tgaattgggg atcctctaca caagccctga
cttacctgaa tgcactgcag cattcaattc gtctttctgg agtggaagac cacgtgaaaa
actttaggcc acagtgtctt gttatgacag gtgctccaaa ctcacgtcca gctttacttc
atcttgttca tgatttcaca aaaaatgttg gtttgatgat ctgtggccat gtacatatgg
gtcctcgaag acaagccatg aaagagatgt ccatcgatca agccaaatat cagcgatggc
ttattaagaa caaaatgaag gcattttatg ctccagtaca tgcagatgac ttgagagaag
gtgcacagta tttgatgcag gctgctggtc ttggtcgtat gaagccaaac acacttgtcc
ttggatttaa gaaagattgg ttgcaagcag atatgaggga tgtggatatg tatataaact
tatttcatga tgcttttgac atacaatatg gagtagtggt tattcgccta aaagaaggtc
tggatatatc tcatcttcaa ggacaagaag aattattgtc atcacaagag aaatctcctg
gcaccaagga tgtggtagta agtgtggaat atagtaaaaa gtccgattta gatacttcca
aaccactcag tgaaaaacca attacacaca aagttgagga agaggatggc aagactgcaa
ctcaaccact gttgaaaaaa gaatccaaag gccctattgt gcctttaaat gtagctgacc
aaaagcttct tgaagctagt acacagtttc agaaaaaaca aggaaagaat actattgatg
tctggtggct ttttgatgat ggaggtttga ccttattgat accttacctt ctgacgacca
agaaaaaatg gaaagactgt aagatcagag tattcattgg tggaaagata aacagaatag
accatgaccg gagagcgatg gctactttgc ttagcaagtt ccggatagac ttttctgata
tcatggttct aggagatatc aataccaaac caaagaaaga aaatattata gcttttgagg
aaatcattga gccatacaga cttcatgaag atgataaaga gcaagatatt gcagataaaa
tgaaagaaga tgaaccatgg cgaataacag ataatgagct tgaactttat aagaccaaga
cataccggca gatcaggtta aatgagttat taaaggaaca ttcaagcaca gctaatatta
ttgtcatgag tctcccagtt gcacgaaaag gtgctgtgtc tagtgctctc tacatggcat
ggttagaagc tctatctaag gacctaccac caatcctcct agttcgtggg aatcatcaga
gtgtccttac cttctattca taa

In one embodiment, NKCC is depleted from the cell's genome, or KCC2, optimized Gi-DREAD described herein, or Kir2.1 is upmodulated in the cell's genome, using any genome editing system including, but not limited to, zinc finger nucleases, TALENS, meganucleases, and CRISPR/Cas systems. In one embodiment, the genomic editing system used to incorporate the nucleic acid encoding one or more guide RNAs into the cell's genome is not a CRISPR/Cas system; this can prevent undesirable cell death in cells that retain a small amount of Cas enzyme/protein. It is also contemplated herein that either the Cas enzyme or the sgRNAs are each expressed under the control of a different inducible promoter, thereby allowing temporal expression of each to prevent such interference.

When a nucleic acid encoding one or more sgRNAs and a nucleic acid encoding an RNA-guided endonuclease each need to be administered in vivo, the use of an adenovirus associated vector (AAV) is specifically contemplated. Other vectors for simultaneously delivering nucleic acids to both components of the genome editing/fragmentation system (e.g., sgRNAs, RNA-guided endonuclease) include lentiviral vectors, such as Epstein Barr, Human immunodeficiency virus (HIV), and hepatitis B virus (HBV). Each of the components of the RNA-guided genome editing system (e.g., sgRNA and endonuclease) can be delivered in a separate vector as known in the art or as described herein.

In one embodiment, the agent inhibits NKCC by RNA inhibition (RNAi). Inhibitors of the expression of a given gene can be an inhibitory nucleic acid. In some embodiments of any of the aspects, the inhibitory nucleic acid is an inhibitory RNA (iRNA). The RNAi can be single stranded or double stranded.

The iRNA can be siRNA, shRNA, endogenous microRNA (miRNA), or artificial miRNA. In one embodiment, an iRNA as described herein effects inhibition of the expression and/or activity of a target, e.g. NKCC. In some embodiments of any of the aspects, the agent is siRNA that inhibits NKCC. In some embodiments of any of the aspects, the agent is shRNA that inhibits NKCC.

One skilled in the art would be able to design siRNA, shRNA, or miRNA to target the nucleic acid sequence of NKCC (e.g., SEQ ID NO: 4), e.g., using publically available design tools. siRNA, shRNA, or miRNA is commonly made using companies such as Dharmacon (Layfayette, Colo.) or Sigma Aldrich (St. Louis, Mo.).

In some embodiments of any of the aspects, the iRNA can be a dsRNA. A dsRNA includes two RNA strands that are sufficiently complementary to hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of an mRNA formed during the expression of the target. The other strand (the sense strand) includes a region that is complementary to the antisense strand, such that the two strands hybridize and form a duplex structure when combined under suitable conditions

The RNA of an iRNA can be chemically modified to enhance stability or other beneficial characteristics. The nucleic acids featured in the invention may be synthesized and/or modified by methods well established in the art, such as those described in “Current protocols in nucleic acid chemistry,” Beaucage, S. L. et al. (Edrs.), John Wiley & Sons, Inc., New York, N.Y., USA, which is hereby incorporated herein by reference.

In one embodiment, the agent is miRNA that inhibits NKCC. microRNAs are small non-coding RNAs with an average length of 22 nucleotides. These molecules act by binding to complementary sequences within mRNA molecules, usually in the 3′ untranslated (3′UTR) region, thereby promoting target mRNA degradation or inhibited mRNA translation. The interaction between microRNA and mRNAs is mediated by what is known as the “seed sequence”, a 6-8-nucleotide region of the microRNA that directs sequence-specific binding to the mRNA through imperfect Watson-Crick base pairing. More than 900 microRNAs are known to be expressed in mammals. Many of these can be grouped into families on the basis of their seed sequence, thereby identifying a “cluster” of similar microRNAs. A miRNA can be expressed in a cell, e.g., as naked DNA. A miRNA can be encoded by a nucleic acid that is expressed in the cell, e.g., as naked DNA or can be encoded by a nucleic acid that is contained within a vector.

The agent may result in gene silencing of the target gene (e.g., NKCC), such as with an RNAi molecule (e.g. siRNA or miRNA). This entails a decrease in the mRNA level in a cell for a target by at least about 5%, about 10%, about 20%, about 30%, about 40%, about 50%, about 60%, about 70%, about 80%, about 90%, about 95%, about 99%, about 100% of the mRNA level found in the cell without the presence of the agent. In one preferred embodiment, the mRNA levels are decreased by at least about 70%, about 80%, about 90%, about 95%, about 99%, about 100%. One skilled in the art will be able to readily assess whether the siRNA, shRNA, or miRNA effective target e.g., NKCC, for its downregulation, for example by transfecting the siRNA, shRNA, or miRNA into cells and detecting the levels of a gene (e.g., NKCC) found within the cell via western-blotting.

The agent may be contained in and thus further include a vector. Many such vectors useful for transferring exogenous genes into target mammalian cells are available. The vectors may be episomal, e.g. plasmids, virus-derived vectors such cytomegalovirus, adenovirus, etc., or may be integrated into the target cell genome, through homologous recombination or random integration, e.g. retrovirus-derived vectors such as MMLV, HIV-1, ALV, etc. In some embodiments, combinations of retroviruses and an appropriate packaging cell line may also find use, where the capsid proteins will be functional for infecting the target cells. Usually, the cells and virus will be incubated for at least about 24 hours in the culture medium. The cells are then allowed to grow in the culture medium for short intervals in some applications, e.g. 24-73 hours, or for at least two weeks, and may be allowed to grow for five weeks or more, before analysis. Commonly used retroviral vectors are “defective”, i.e. unable to produce viral proteins required for productive infection. Replication of the vector requires growth in the packaging cell line.

The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, artificial chromosome, virus, virion, etc.

As used herein, the term “expression vector” refers to a vector that directs expression of an RNA or polypeptide (e.g., KCC2, Gi-DREADD, or Kir2.1) from nucleic acid sequences contained therein linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

Integrating vectors have their delivered RNA/DNA permanently incorporated into the host cell chromosomes. Non-integrating vectors remain episomal which means the nucleic acid contained therein is never integrated into the host cell chromosomes. Examples of integrating vectors include retroviral vectors, lentiviral vectors, hybrid adenoviral vectors, and herpes simplex viral vector.

One example of a non-integrative vector is a non-integrative viral vector. Non-integrative viral vectors eliminate the risks posed by integrative retroviruses, as they do not incorporate their genome into the host DNA. One example is the Epstein Barr oriP/Nuclear Antigen-1 (“EBNA1”) vector, which is capable of limited self-replication and known to function in mammalian cells. As containing two elements from Epstein-Barr virus, oriP and EBNA1, binding of the EBNA1 protein to the virus replicon region oriP maintains a relatively long-term episomal presence of plasmids in mammalian cells. This particular feature of the oriP/EBNA1 vector makes it ideal for generation of integration-free iPSCs. Another non-integrative viral vector is adenoviral vector and the adeno-associated viral (AAV) vector.

Another non-integrative viral vector is RNA Sendai viral vector, which can produce protein without entering the nucleus of an infected cell. The F-deficient Sendai virus vector remains in the cytoplasm of infected cells for a few passages, but is diluted out quickly and completely lost after several passages (e.g., 10 passages).

Another example of a non-integrative vector is a minicircle vector. Minicircle vectors are circularized vectors in which the plasmid backbone has been released leaving only the eukaryotic promoter and cDNA(s) that are to be expressed.

In various embodiments, the vector crosses the blood brain barrier. In other embodiments, any agent described herein is formulated to cross the blood brain barrier. The blood brain barrier is a highly selective semipermeable membrane barrier that separates the circulating blood from the brain extracellular fluid in the central nervous system (CNS). For therapeutics needed to be delivered to the CNS, a skilled clinician can directly deliver a therapeutic to the spinal canal. For direct administration into the spinal canal, the compounds and compositions described herein will be administered via intrathecal administration by a skilled clinician. Intrathecal administration is a route of drug administration in which the drug is directly injected in the spinal cancal or in the subarachnoid space, allowing it to directly reach the cerebrospinal fluid (CSF). Non-limiting examples of other drugs that are administered via intrathecal administration are spinal anesthesia, chemotherapeutics, pain management drugs, and therapeutics that cannot pass the blood brain barrier. A vector can be packaged with at least a second agent that permabilizes the blood brain barrier. One skilled in the art can determine if a vector has crossed the blood brain barrier, e.g., by determining if the vector is detected in, e.g., spinal fluid, following administration.

Pharmaceutical Compositions

Compositions described herein at directed for the use in treating a spinal cord injury. Modes for administration for these compositions are further described herein below. In various embodiment, any pharmaceutical composition described herein further comprises at least a second therapeutic compound. In one embodiment, the second therapeutic compound is useful for the treatment of a spinal cord injury.

One aspect of the invention provides a pharmaceutical composition comprising an effective amount of KCC2 polypeptide or a vector comprising a nucleic acid sequence encoding the KCC2 polypeptide and a pharmaceutically acceptable carrier, for use in treating spinal cord injury. In one embodiment, the KCC2 polypeptide comprises the nucleic acid sequence of a mammalian KCC2, e.g, rat KCC2.

In one embodiment, the KCC2 polypeptide comprises the sequence of SEQ ID NO: 1.

SEQ ID NO:1 is a nucleic acid sequence encoding rat KCC2.

(SEQ ID NO: 1)
ATGCTCAACAACCTGACGGACTGCGAGGACGG
CGATGGGGGAGCCAACCCGGGTGACGGCAATC
CCAAGGAGAGCAGCCCCTTCATCAACAGCACG
GACACGGAGAAGGGGAGAGAGTATGATGGCAG
GAACATGGCCCTGTTTGAGGAGGAGATGGACA
CCAGCCCCATGGTATCCTCCCTGCTCAGTGGG
CTGGCCAACTACACCAACCTGCCTCAGGGAAG
CAAAGAGCACGAAGAAGCAGAAAACAATGAGG
GCGGAAAGAAGAAGCCGGTGCAGGCCCCACGC
ATGGGCACCTTCATGGGCGTGTACCTCCCGTG
CCTGCAGAACATCTTTGGTGTTATCCTCTTTC
TGCGGCTCACTTGGGTGGTGGGAATCGCAGGC
ATCATGGAGTCCTTCTGCATGGTCTTCATCTG
CTGCTCCTGCACGATGCTCACAGCCATTTCCA
TGAGCGCAATTGCAACCAATGGTGTTGTGCCT
GCTGGTGGCTCCTACTACATGATTTCCAGGTC
TCTGGGCCCGGAGTTTGGGGGCGCCGTGGGCC
TCTGCTTCTACCTGGGCACTACCTTTGCTGGG
GCTATGTACATCCTGGGCACCATCGAGATCCT
GCTGGCTTACCTCTTCCCAGCGATGGCCATCT
TCAAGGCAGAAGATGCCAGTGGGGAGGCAGCC
GCCATGTTGAATAACATGCGGGTGTATGGCAC
CTGTGTGCTCACCTGCATGGCCACCGTAGTCT
TTGTGGGCGTCAAGTACGTGAACAAGTTTGCC
CTGGTCTTCCTGGGTTGCGTGATCCTCTCCAT
CCTGGCCATCTACGCAGGGGTCATCAAGTCTG
CCTTCGATCCACCCAATTTCCCGATTTGCCTC
CTGGGGAACCGCACGCTGTCTCGCCATGGCTT
TGATGTCTGTGCCAAGCTGGCTTGGGAAGGAA
ATGAGACAGTGACCACACGGCTCTGGGGCCTA
TTCTGTTCCTCCCGCCTCCTCAATGCCACCTG
TGATGAGTACTTCACCCGAAACAATGTCACAG
AGATCCAGGGCATTCCTGGTGCTGCAAGTGGC
CTCATCAAAGAGAACCTGTGGAGTTCCTACCT
GACCAAGGGGGTGATCGTGGAGAGGCGTGGGA
TGCCCTCTGTGGGCCTGGCAGATGGTACCCCC
GTTGACATGGACCACCCCTATGTCTTCAGTGA
TATGACCTCCTACTTCACCCTGCTTGTTGGCA
TCTATTTCCCCTCAGTCACAGGGATCATGGCT
GGCTCGAACCGGTCCGGAGACCTGCGGGATGC
CCAGAAGTCTATCCCTACTGGAACTATCTTGG
CCATTGCTACGACCTCTGCTGTCTACATCAGC
TCTGTTGTTCTGTTCGGAGCCTGCATCGAAGG
GGTCGTCCTACGGGACAAGTTTGGGGAAGCTG
TGAATGGCAATCTGGTGGTGGGCACCCTGGCC
TGGCCTTCTCCTTGGGTCATTGTCATAGGCTC
TTTCTTCTCTACCTGCGGAGCTGGACTACAGA
GCCTCACAGGGGCCCCACGCCTGCTGCAGGCC
ATCTCCCGGGATGGCATAGTGCCCTTCCTGCA
GGTCTTTGGCCATGGCAAAGCCAACGGAGAGC
CAACCTGGGCGCTGCTGCTGACTGCCTGCATC
TGTGAGATCGGCATCCTCATCGCCTCCCTGGA
TGAGGTCGCCCCTATCCTTTCCATGTTCTTCC
TGATGTGTTACATGTTTGTGAACTTGGCTTGC
GCGGTGCAGACACTGCTGAGGACGCCCAACTG
GAGGCCACGCTTCCGATATTACCACTGGACCC
TCTCCTTCCTGGGCATGAGCCTCTGCCTGGCC
CTGATGTTCATTTGCTCCTGGTATTATGCGCT
GGTAGCTATGCTCATCGCTGGCCTCATCTATA
AGTACATCGAGTACCGGGGGGCAGAGAAGGAG
TGGGGGGATGGGATCCGAGGCCTGTCTCTCAG
TGCAGCTCGCTATGCTCTCTTGCGTCTGGAGG
AAGGACCCCCGCATACAAAGAACTGGAGGCCC
CAGCTACTGGTGCTGGTGCGTGTGGACCAGGA
CCAGAACGTGGTGCACCCGCAGCTGCTGTCCT
TGACCTCCCAGCTCAAGGCAGGGAAGGGCCTG
ACCATTGTGGGCTCTGTCCTTGAGGGCACCTT
TCTGGACAACCACCCTCAGGCTCAGCGGGCAG
AGGAGTCTATCCGGCGCCTGATGGAGGCTGAG
AAGGTGAAGGGCTTCTGCCAGGTAGTGATCTC
CTCCAACCTGCGTGACGGTGTGTCCCACCTGA
TCCAATCCGGGGGCCTCGGGGGCCTGCAACAC
AACACTGTGCTAGTGGGCTGGCCTCGCAACTG
GCGACAGAAGGAGGATCATCAGACATGGAGGA
ACTTCATCGAACTCGTCCGGGAAACTACAGCT
GGCCACCTCGCCCTGCTGGTCACCAAGAATGT
TTCCATGTTCCCCGGGAACCCTGAGCGTTTCT
CTGAGGGCAGCATTGACGTGTGGTGGATCGTG
CACGACGGGGGCATGCTCATGCTGTTGCCCTT
CCTCCTGCGTCACCACAAGGTCTGGAGGAAAT
GCAAAATGCGGATCTTCACCGTGGCGCAGATG
GATGACAACAGCATTCAGATGAAGAAAGACCT
GACCACGTTTCTGTACCACTTACGAATTACTG
CAGAGGTGGAAGTCGTGGAGATGCACGAGAGC
GACATCTCAGCATACACCTACGAGAAGACATT
GGTAATGGAACAACGTTCTCAGATCCTCAAAC
AGATGCACCTCACCAAGAACGAGCGGGAACGG
GAGATCCAGAGCATCACAGATGAATCTCGGGG
CTCCATTCGGAGGAAGAATCCAGCCAACACTC
GGCTCCGCCTCAATGTTCCCGAAGAGACAGCT
TGTGACAACGAGGAGAAGCCAGAAGAGGAGGT
GCAGCTGATCCATGACCAGAGTGCTCCCAGCT
GCCCTAGCAGCTCGCCGTCTCCAGGGGAGGAG
CCTGAGGGGGAGGGGGAGACAGACCCAGAGAA
GGTGCATCTCACCTGGACCAAGGATAAGTCAG
CGGCTCAGAAGAACAAAGGCCCCAGTCCCGTC
TCCTCGGAGGGGATCAAGGACTTCTTCAGCAT
GAAGCCGGAGTGGGAAAACTTGAACCAGTCCA
ACGTGCGGCGCATGCACACAGCTGTGCGGCTG
AACGAGGTCATCGTGAATAAATCCCGGGATGC
CAAGTTGGTGTTGCTCAACATGCCCGGGCCTC
CCCGCAACCGCAATGGAGATGAAAACTACATG
GAGTTCCTGGAGGTCCTCACTGAGCAACTGGA
CCGGGTGATGCTGGTCCGCGGTGGTGGCCGAG
AGGTCATCACCATCTACTCCTGA

In one embodiment, the KCC2 polypeptide has, comprises, consists of, or consists essentially of at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more amino acid sequence identity to SEQ ID NO: 1 and retains at least 80% of the biological activity of KCC2 of SEQ ID NO: 1. As used herein, biological activity of KCC2 refers to, but is not limited to, its function to mediate the potassium and chloride gradient.

Another aspect of the invention provides a pharmaceutical composition comprising an effective amount of Gi-DREADD polypeptide or a vector comprising a nucleic acid sequence the Gi-DREADD polypeptide and a pharmaceutically acceptable carrier, for use in treating spinal cord injury. In one embodiment, the Gi-DREADD polypeptide is optimized for expression in the inhibitory interneurons. In one embodiment, the composition further comprises clozapine N-oxide.

In one embodiment, the Gi-DREADD polypeptide comprises the sequence of SEQ ID NO: 2.

SEQ ID NO: 2 is a nucleic acid sequence encoding optimized Gi-DREADD.

(SEQ ID NO: 2)
ATGGCCAACT TCACACCTGT CAATGGCAGC TCGGGCAATC
AGTCCGTGCG CCTGGTCACG TCATCATCCC
ACAATCGCTA TGAGACGGTG GAAATGGTCT TCATTGCCAC
AGTGACAGGC TCCCTGAGCC TGGTGACTGT
CGTGGGCAAC ATCCTGGTGA TGCTGTCCAT CAAGGTCAAC
AGGCAGCTGC AGACAGTCAA CAACTACTTC
CTCTTCAGCC TGGCGTGTGC TGATCTCATC ATAGGCGCCT
TCTCCATGAA CCTCTACACC GTGTACATCA
TCAAGGGCTA CTGGCCCCTG GGCGCCGTGG TCTGCGACCT
GTGGCTGGCC CTGGACTGCG TGGTGAGCAA
CGCCTCCGTC ATGAACCTTC TCATCATCAG CTTTGACCGC
TACTTCTGCG TCACCAAGCC TCTCACCTAC
CCTGCCCGGC GCACCACCAA GATGGCAGGC CTCATGATTG
CTGCTGCCTG GGTACTGTCC TTCGTGCTCT
GGGCGCCTGC CATCTTGTTC TGGCAGTTTG TGGTGGGTAA
GCGGACGGTG CCCGACAACC AGTGCTTCAT
CCAGTTCCTG TCCAACCCAG CAGTGACCTT TGGCACAGCC
ATTGCTGGCT TCTACCTGCC TGTGGTCATC
ATGACGGTGC TGTACATCCA CATCTCCCTG GCCAGTCGCA
GCCGAGTCCA CAAGCACCGG CCCGAGGGCC
CGAAGGAGAA GAAAGCCAAG ACGCTGGCCT TCCTCAAGAG
CCCACTAATG AAGCAGAGCG TCAAGAAGCC
CCCGCCCGGG GAGGCCGCCC GGGAGGAGCT GCGCAATGGC
AAGCTGGAGG AGGCCCCCCC GCCAGCGCTG
CCACCGCCAC CGCGCCCCGT GGCTGATAAG GACACTTCCA
ATGAGTCCAG CTCAGGCAGT GCCACCCAGA
ACACCAAGGA ACGCCCAGCC ACAGAGCTGT CCACCACAGA
GGCCACCACG CCCGCCATGC CCGCCCCTCC
CCTGCAGCCG CGGGCCCTCA ACCCAGCCTC CAGATGGTCC
AAGATCCAGA TTGTGACGAA GCAGACAGGC
AATGAGTGTG TGACAGCCAT TGAGATTGTG CCTGCCACGC
CGGCTGGCAT GCGCCCTGCG GCCAACGTGG
CCCGCAAGTT CGCCAGCATC GCTCGCAACC AGGTGCGCAA
GAAGCGGCAG ATGGCGGCCC GGGAGCGCAA
AGTGACACGA ACGATCTTTG CCATTCTGCT GGCCTTCATC
CTCACCTGGA CGCCCTACAA CGTCATGGTC
CTGGTGAACA CCTTCTGCCA GAGCTGCATC CCTGACACGG
TGTGGTCCAT TGGCTACTGG CTCTGCTACG
TCAACAGCAC CATCAACCCT GCCTGCTATG CTCTGTGCAA
CGCCACCTTT AAAAAGACCT TCCGGCACCT
GCTGCTGTGC CAGTATCGGA ACATCGGCAC TGCCAGGCG

In one embodiment of any aspect, the Gi-DREADD polypeptide has, comprises, consists of, or consists essentially of at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more amino acid sequence identity to SEQ ID NO: 2 and retains at least 80% of the biological activity of Gi-DREADD of SEQ ID NO: 2.

Yet another aspect of the invention provides a pharmaceutical composition comprising an effective amount of Kir2.1 polypeptide or a vector comprising an amino acid sequence encoding the Kir2.1 polypeptide and a pharmaceutically acceptable carrier, for use in treating spinal cord injury.

In one embodiment, the Kir2.1 polypeptide comprises the sequence of SEQ ID NO: 3.

SEQ ID NO: 3 is an amino acid sequence encoding human Kir2.1 polypeptide.

(SEQ ID NO: 3)
MGSVRTNRYSIVSSEEDGMKLATMAVANGFGNGKSKVHTRQQCRSRFVK
KDGHCNVQFINVGEKGQRYLADIFTTCVDIRWRWMLVIFCLAFVLSWLF
FGCVFWLIALLHGDLDASKEGKACVSEVNSFTAAFLFSIETQTTIGYGF
RCVTDECPIAVFMVVFQSIVGCIIDAFIIGAVMAKMAKPKKRNETLVFS
HNAVIAMRDGKLCLMWRVGNLRKSHLVEAHVRAQLLKSRITSEGEYIPL
DQIDINVGFDSGIDRIFLVSPITIVHEIDEDSPLYDLSKQDIDNADFEI
VVILEGMVEATAMTTQCRSSYLANEILWGHRYEPVLFEEKHYYKVDYSR
FHKTYEVPNTPLCSARDLAEKKYILSNANSFCYENEVALTSKEEDDSEN
GVPESTSTDTPPDIDLHNQASVPLEPRPLRRESEI

In one embodiment, the Kir2.1 polypeptide has, comprises, consists of, or consists essentially of at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more amino acid sequence identity to SEQ ID NO: 3 and retains at least 80% of the biological activity of Kir2.1 of SEQ ID NO: 3.

In one embodiment, the Kir2.1 polypeptide comprises the sequence of SEQ ID NO: 5.

SEQ ID NO: 5 is an amino acid sequence encoding mouse Kir2.1 polypeptide.

(SEQ ID NO: 5)
MGSVRTNRYSIVSSEEDGMKLATMAVANGFGNGKSKVHTRQQCRSRFVK
KDGHCNVQFINVGEKGQRYLADIFTTCVDIRWRWMLVIFCLAFVLSWLF
FGCVFWLIALLHGDLDTSKVSKACVSEVNSFTAAFLFSIETQTTIGYGF
RCVTDECPIAVFMVVFQSIVGCIIDAFIIGAVMAKMAKPKKRNETLVFS
HNAVIAMRDGKLCLMWRVGNLRKSHLVEAHVRAQLLKSRITSEGEYIPL
DQIDINVGFDSGIDRIFLVSPITIVHEIDEDSPLYDLSKQDIDNADFEI
VVILEGMVEATAMTTQCRSSYLANEILWGHRYEPVLFEEKHYYKVDYSR
FHKTYEVPNTPLCSARDLAEKKYILSNANSFCYENEVALTSKEEEEDSE
NGVPESTSTDSPPGIDLHNQASVPLEPRPLRRESEI

In one embodiment, the Kir2.1 polypeptide has, comprises, consists of, or consists essentially of at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more amino acid sequence identity to SEQ ID NO: 5 and retains at least 80% of the biological activity of Kir2.1 of SEQ ID NO: 5.

Another aspect of the invention provides a pharmaceutical composition comprising an effective amount of any of the agents that inhibit NKCC described herein and a pharmaceutically acceptable carrier, for use in treating spinal cord injury. In one embodiment of any aspect, the composition further comprises at least a second therapeutic compound.

In one embodiment, a composition comprises any agent described herein that modulates KCC2, NKCC, optimized Gi-DREAD described herein, or Kir2.1.

As used here, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include, but are not limited to: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, binding agents, fillers, lubricants, coloring agents, disintegrants, release agents, coating agents, sweetening agents, flavoring agents, perfuming agents, preservative, water, salt solutions, alcohols, antioxidants, polyethylene glycols, gelatin, lactose, amylose, magnesium stearate, talc, silicic acid, viscous paraffin, hydroxymethylcellulose, polyvinylpyrrolidone and the like can also be present in the formulation. The terms such as “excipient”, “carrier”, “pharmaceutically acceptable carrier” or the like are used interchangeably herein.

In one aspect described herein, a composition described herein further comprises an agent that facilitates passage through the blood brain barrier. In one embodiment, the pharmaceutically acceptable facilitates the passage through, or has the capacity to pass through the blood brain barrier.

Administration

In some embodiments, the methods described herein relate to treating a subject having or diagnosed as having a spinal cord injury comprising administering an agent that upmodulates KCC2 as described herein. In some embodiments, the methods described herein relate to treating a subject having or diagnosed as having a spinal cord injury comprising administering an agent that inhibits NKCC as described herein. In some embodiments, the methods described herein relate to treating a subject having or diagnosed as having a spinal cord injury comprising administering an agent that upmodulates Gi-DREADD as described herein. In some embodiments, the methods described herein relate to treating a subject having or diagnosed as having a spinal cord injury comprising administering an agent that upmodulates Kir2.1 as described herein. Subjects having a spinal cord injury can be identified by a physician using current methods of diagnosing a condition. Symptoms and/or complications of a spinal cord injury, which characterize this injury and aid in diagnosis are well known in the art and include but are not limited to, loss or reduce mobility in limbs. Tests that may aid in a diagnosis of, e.g. a spinal cord injury, include but are not limited to an x-ray, an MRI scan, or a CT scan.

The agents described herein (e.g., an agent that upmodulates KCC2, Gi-DREADD, e.g., optimized Gi-DREADD as described herein), or Kir2.1, or an agent that inhibits NKCC) can be administered to a subject having or diagnosed as having a spinal cord injury. In some embodiments, the methods described herein comprise administering an effective amount of an agent to a subject in order to alleviate at least one symptom of the spinal cord injury. As used herein, “alleviating at least one symptom of the spinal cord injury” is ameliorating any condition or symptom associated with the spinal cord injury (e.g., loss of feeling or mobility in limbs). As compared with an equivalent untreated control, such reduction is by at least 5%, 10%, 20%, 40%, 50%, 60%, 80%, 90%, 95%, 99% or more as measured by any standard technique. A variety of means for administering the agents described herein to subjects are known to those of skill in the art. In one embodiment, the agent is administered systemically or locally (e.g., to the spinal cord, or at the site of injury on the spinal cord). In one embodiment, the agent is administered intravenously. In one embodiment, the agent is administered continuously, in intervals, or sporadically. The route of administration of the agent will be optimized for the type of agent being delivered (e.g., an antibody, a small molecule, an RNAi), and can be determined by a skilled practitioner.

The term “effective amount” as used herein refers to the amount of an agent (e.g., an agent that upmodulates KCC2, Gi-DREADD, or Kir2.1, or an agent that inhibits NKCC) can be administered to a subject having or diagnosed as having a spinal cord injury needed to alleviate at least one or more symptom of a spinal cord injury. The term “therapeutically effective amount” therefore refers to an amount of an agent that is sufficient to provide a particular anti-spinal cord injury effect when administered to a typical subject. An effective amount as used herein, in various contexts, would also include an amount of an agent sufficient to delay the development of a symptom of a spinal cord injury, alter the course of a symptom of a spinal cord injury (e.g., slowing the progression of loss of feeling or mobility in limbs), or reverse a symptom of a spinal cord injury (e.g., restoring feeling or mobility in limbs that was previously reduced or lost). Thus, it is not generally practicable to specify an exact “effective amount”. However, for any given case, an appropriate “effective amount” can be determined by one of ordinary skill in the art using only routine experimentation.

In one embodiment, the agent is administered within at least 1 minute, at least 2 minutes, at least 3 minutes, at least 4 minutes, at least 5 minutes, at least 10 minutes, at least 15 minutes, at least 20 minutes, at least 25 minutes, at least 30 minutes, at least 35 minutes, at least 40 minutes, at least 45 minutes, at least 50 minutes, at least 55 minutes, at least 1 hour, at least 2 hours, at least 3 hours, at least 4 hours, at least 5 hours, at least 6 hours, at least 12 hours, at least 18 hours, at least 24 hours, at least 36 hours, at least 48 hours, at least 60 hours, at least 72 hours, at least 96 hours, at least 5 days, at least 6 days, at least 1 week, at least 2 weeks, at least 3 weeks, at least 1 month, at least 2 months, at least 3 months, at least 4 months, at least 5 months, at least 6 months, at least 7 months, at least 8 months, at least 9 months, at least 10 months, at least 11 months, at least 12 months, at least 2 years, at least 3 years, at least 4 years, or at least 5 years or more following the occurrence of the spinal cord injury.

In one embodiment, the agent can be used in an amount of about 0.001 to 25 mg/kg of body weight or about 0.005 to 8 mg/kg of body weight or about 0.01 to 6 mg/kg of body weight or about 0.1 to 0.2 mg/kg of body weight or about 1 to 2 mg/kg of body weight. In some embodiments, the agent can be used in an amount of about 0.1 to 1000 μg/kg of body weight or about 1 to 100 μg/kg of body weight or about 10 to 50 μg/kg of body weight. In one embodiment, the agent is used in an amount ranging from 0.01 μg to 15 mg/kg of body weight per dose, e.g., 10, 1, 0.1, 0.01, 0.001, or 0.00001 mg per kg of bodyweight per dose. [Inventors-what does range would you expect to use?]

Effective amounts, toxicity, and therapeutic efficacy can be evaluated by standard pharmaceutical procedures in cell cultures or experimental animals. The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of the agent, which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., measuring mobility of limbs, measuring reflexes, among others. The dosage can be determined by a physician and adjusted, as necessary, to suit observed effects of the treatment.

Dosage

“Unit dosage form” as the term is used herein refers to a dosage for suitable one administration. By way of example a unit dosage form can be an amount of therapeutic disposed in a delivery device, e.g., a syringe or intravenous drip bag. In one embodiment, a unit dosage form is administered in a single administration. In another, embodiment more than one unit dosage form can be administered simultaneously.

The dosage of the agent as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to administer further cells, discontinue treatment, resume treatment, or make other alterations to the treatment regimen. The dosage should not be so large as to cause adverse side effects, such as cytokine release syndrome. Generally, the dosage will vary with the age, condition, and sex of the patient and can be determined by one of skill in the art. The dosage can also be adjusted by the individual physician in the event of any complication.

Combinational Therapy

In one embodiment, the agent described herein is used as a monotherapy. In one embodiment, the agents described herein can be used in combination with other known agents and therapies for a spinal cord injury. Administered “in combination,” as used herein, means that two (or more) different treatments are delivered to the subject during the course of the subject's affliction with the injury, e.g., the two or more treatments are delivered after the subject has been diagnosed with the injury and before the injury has been cured or eliminated or treatment has ceased for other reasons. In some embodiments, the delivery of one treatment is still occurring when the delivery of the second begins, so that there is overlap in terms of administration. This is sometimes referred to herein as “simultaneous”, “at substantially the same time” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the injury is greater than what would be observed with one treatment delivered in the absence of the other. The effect of the two treatments can be partially additive, wholly additive, or greater than additive. The delivery can be such that an effect of the first treatment delivered is still detectable when the second is delivered. The agents described herein and the at least one additional therapy can be administered simultaneously, in the same or in separate compositions, or sequentially. For sequential administration, the agent described herein can be administered first, and the additional agent can be administered second, or the order of administration can be reversed. The agent can be administered before another treatment, concurrently with the treatment, post-treatment, or during remission of the disorder.

Treatments currently used to treat spinal cord injury include, but are not limited to, physical therapy, electrostimulation, surgery to repair damaged spinal cord, stem cell therapy, hyperbaric oxygen therapy. Pharmalogical treatments used to treat spinal cord injury include, but are not limited to, corticosteroids (e.g., dexamethasone and methylprednisolone), gangliosides, Tirilazad, Naloxone.

Additional compounds that can be administered with the agents described herein include, but are not limited to axon regeneration promoters (such as osteopontin, and growth factors), and 4-aminopuridine.

Osteopontin, also known as bone sialoprotein I (BSP-1 or BNSP), early T-lymphocyte activation (ETA-1), secreted phosphoprotein 1 (SPP1), 2ar, and Rickettsia resistance (Ric), is encoded by the secreted phosphoprotein 1 (SPP1) gene. Osteopontin is expressed in, for example bine, and functions as an extracellular structural protein. Sequences for Osteopontin (OPN) are known in the art for a number of species, e.g., human Osteopontin (NCBI Gene ID: 6696) polypeptide (e.g., NCBI Ref Seq NP_000573.1) and mRNA (e.g., NCBI Ref Seq NM_000582.2). Osteopontin can refer to human Osteopontin, including naturally occurring variants, molecules, and alleles thereof. Osteopontin refers to the mammalian Osteopontin of, e.g., mouse, rat, rabbit, dog, cat, cow, horse, pig, and the like. Administration of Osteopontin is described in, for example, international application number WO/1999033415, US2004/0142865, and WO/2003046135; or US application number U.S. Ser. No. 11/936,623; or U.S. Pat. No. 6,686,444 or 5,695,761; the contents of which are each incorporated herein by reference in their entireties.

4-aminopuridine, a prescription muscle strengthener, is also known in the art as C5H4N—NH2, and has a structure of

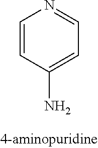

When administered in combination, the agent and the additional agent (e.g., second or third agent), or all, can be administered in an amount or dose that is higher, lower or the same as the amount or dosage of each agent used individually, e.g., as a monotherapy. In certain embodiments, the administered amount or dosage of the agent, the additional agent (e.g., second or third agent), or all, is lower (e.g., at least 20%, at least 30%, at least 40%, or at least 50%) than the amount or dosage of each agent used individually. In other embodiments, the amount or dosage of agent, the additional agent (e.g., second or third agent), or all, that results in a desired effect (e.g., treatment of a spinal cord injury) is lower (e.g., at least 20%, at least 30%, at least 40%, or at least 50% lower) than the amount or dosage of each agent individually required to achieve the same therapeutic effect.

Parenteral Dosage Forms

Parenteral dosage forms of an agents described herein can be administered to a subject by various routes, including, but not limited to, epidural injection, subcutaneous, intravenous (including bolus injection), intramuscular, and intraarterial. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, controlled-release parenteral dosage forms, and emulsions.

Suitable vehicles that can be used to provide parenteral dosage forms of the disclosure are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

Controlled and Delayed Release Dosage Forms

In some embodiments of the aspects described herein, an agent is administered to a subject by controlled- or delayed-release means. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. (Kim, Cherng-ju, Controlled Release Dosage Form Design, 2 (Technomic Publishing, Lancaster, Pa.: 2000)). Controlled-release formulations can be used to control a compound of formula (I)'s onset of action, duration of action, plasma levels within the therapeutic window, and peak blood levels. In particular, controlled- or extended-release dosage forms or formulations can be used to ensure that the maximum effectiveness of an agent is achieved while minimizing potential adverse effects and safety concerns, which can occur both from under-dosing a drug (i.e., going below the minimum therapeutic levels) as well as exceeding the toxicity level for the drug.

A variety of known controlled- or extended-release dosage forms, formulations, and devices can be adapted for use with any agent described herein. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185, each of which is incorporated herein by reference in their entireties. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), multilayer coatings, microparticles, liposomes, or microspheres or a combination thereof to provide the desired release profile in varying proportions. Additionally, ion exchange materials can be used to prepare immobilized, adsorbed salt forms of the disclosed compounds and thus effect controlled delivery of the drug. Examples of specific anion exchangers include, but are not limited to, DUOLITE® A568 and DUOLITE® AP143 (Rohm & Haas, Spring House, Pa. USA).

Efficacy

The efficacy of an agent described herein, e.g., for the treatment of a spinal cord injury, can be determined by the skilled practitioner. However, a treatment is considered “effective treatment,” as the term is used herein, if one or more of the signs or symptoms of the spinal cord injury are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of an injury treated according to the methods described herein or any other measurable parameter appropriate, e.g., feeling and/or mobility in limbs. Efficacy can also be measured by a failure of an individual to worsen as assessed by hospitalization, or need for medical interventions (i.e., progression of the loss of feeling or mobility in limbs). Methods of measuring these indicators are known to those of skill in the art and/or are described herein.

Efficacy can be assessed in animal models of a condition described herein, for example, a mouse model or an appropriate animal model of spinal cord injuries, as the case may be. When using an experimental animal model, efficacy of treatment is evidenced when a statistically significant change in a marker is observed, e.g., increased limb mobility following loss of mobility.

All patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The present invention can be defined in any of the following numbered paragraphs:
1) A method for treating a spinal injury, comprising administering to a subject having a spinal injury an effective amount of an agent that upmodulates neuron-specific K⁺—Cl⁻ co-transporter (KCC2).
2) The method of paragraph 1, wherein the agent that upmodulates KCC2 is selected from the group consisting of a small molecule, a peptide, a gene editing system, and an expression vector encoding KCC2.
3) The method of any of the preceding paragraphs, wherein the small molecule is CLP290.
4) The method of any of the preceding paragraphs, wherein the vector is non-integrative or integrative.
5) The method of any of the preceding paragraphs, wherein the vector is a viral vector or non-viral vector.
6) The method of any of the preceding paragraphs, wherein the non-integrative vector is selected from the group consisting of an episomal vector, an EBNA1 vector, a minicircle vector, a non-integrative adenovirus, a non-integrative RNA, and a Sendai virus. 7) The method of any of the preceding paragraphs, wherein the viral vector is selected from the group consisting of retrovirus, lentivirus, adenovirus, herpesvirus, poxvirus, alpha virus, vaccinia virus, and adeno-associated viruses.
8) The method of any of the preceding paragraphs, wherein the non-viral vector is selected from the group consisting of a nanoparticle, a cationic lipid, a cationic polymer, a metallic nanoparticle, a nanorod, a liposome, microbubbles, a cell penetrating peptide and a liposphere.
9) The method of any of the preceding paragraphs, wherein the vector crosses the blood brain barrier.
10) The method of any of the preceding paragraphs, wherein KCC2 is upmodulated by at least 2-fold, 5-fold, 6-fold, 7-fold, 8-fold, 9-fold, or 10-fold as compared to an appropriate control.
11) The methods of any of the preceding paragraphs, wherein the spinal injury is a severe spinal cord injury.
12) The method of any of the preceding paragraphs, wherein the subject is human.
13) The method of any of the preceding paragraphs, wherein the subject has been diagnosed with a spinal injury.
14) The method of any of the preceding paragraphs, wherein the subject has been previously treated for a spinal injury.
15) The method of any of the preceding paragraphs, wherein prior to administering, the subject is diagnosed with having a spinal cord injury.
16) The method of any of the preceding paragraphs, wherein the subject is further administered at least a second spinal injury treatment.
17) The method of any of the preceding paragraphs, wherein the subject is further administered at least a second therapeutic compound.
18) The method of any of the preceding paragraphs, wherein the second therapeutic compound is selected from the group consisting of osteopontin, a growth factor, or 4-aminopuridine.
19) A method for treating a spinal injury, comprising administering to a subject having a spinal injury an effective amount of an agent that inhibits Na+/2Cl−/K+ co-transporter (NKCC).
20) The method of paragraph 19, wherein the agent that inhibits NKCC is selected from the group consisting of a small molecule, an antibody, a peptide, an antisense oligonucleotide, and an RNAi.
21) The method of any of the preceding paragraphs, wherein the RNAi is a microRNA, an siRNA, or an shRNA.
22) The method of any of the preceding paragraphs, wherein the small molecule is bumetanide.
23) The method of any of the preceding paragraphs, wherein the agent is comprised in a vector.
24) A method for treating a spinal injury, comprising administering to a subject having a spinal injury an effective amount of an agent that reduces excitability of inhibitory interneurons.
25) The method of any of the preceding paragraphs, wherein the agent upmodulates the inhibitory Gi-coupled receptor Gi-DREADD.
26) The method of any of the preceding paragraphs, wherein the agent is an expression vector encoding Gi-DREADD.
27) The method of any of the preceding paragraphs, wherein the agent is an expression vector encoding Kir2.1.
28) The method of any of the preceding paragraphs, further comprising administering clozapine N-oxide at substantially the same time as the agent.
29) The method of any of the preceding paragraphs, wherein the vector crosses the blood brain barrier.
30) The method of any of the preceding paragraphs, wherein the excitability of inhibitory interneurons is reduced by at least 10%, at least 20%, at least 30%, at least 40%, at least 50%, at least 60%, at least 70%, at least 80%, at least 90, at least 99%, or more as compared to an appropriate control.
31) The method of any of the preceding paragraphs, wherein prior to administering, the subject is diagnosed with having a spinal cord injury.
32) The method of any of the preceding paragraphs, wherein the subject is administered at least a second spinal injury treatment.
33) A method for treating a spinal injury, comprising administering to a subject having a spinal injury an effective amount electrical stimulation that reduces excitability of inhibitory interneurons.
34) The method of any of the preceding paragraphs, further comprising administering clozapine N-oxide at substantially the same time as the agent.
35) The method of any of the preceding paragraphs, wherein the electrical stimulation is applied directly to the spinal cord.
36) The method of any of the preceding paragraphs, wherein the electrical stimulation is applied directly to the spinal cord at the site of injury.
37) The method of any of the preceding paragraphs, wherein the excitability of inhibitory interneurons is reduced by at least 10%, at least 20%, at least 30%, at least 40%, at least 50%, at least 60%, at least 70%, at least 80%, at least 90, at least 99%, or more as compared to an appropriate control.
38) The method of any of the preceding paragraphs, wherein prior to administering, the subject is diagnosed with having a spinal cord injury.
39) The method of any of the preceding paragraphs, wherein the subject is administered at least a second spinal injury treatment.
40) A pharmaceutical composition comprising an effective amount of a KCC2 polypeptide or a vector comprising a nucleic acid sequence encoding the KCC2 polypeptide and a pharmaceutically acceptable carrier, for use in treating spinal cord injury.
41) The pharmaceutical composition of any of the preceding paragraphs, wherein the KCC2 polypeptide comprises the sequence of SEQ ID NO: 1
42) The pharmaceutical composition of any of the preceding paragraphs, wherein the KCC2 polypeptide has at least 95% amino acid sequence identity to SEQ ID NO: 1 and retains at least 80% of the biological activity of KCC2 of SEQ ID NO: 1.
43) The pharmaceutical composition of any of the preceding paragraphs, further comprising at least a second therapeutic compound.
44) A pharmaceutical composition comprising an effective amount of Gi-DREADD polypeptide or a vector comprising a nucleic acid sequence encoding the Gi-DREADD polypeptide and a pharmaceutically acceptable carrier, for use in treating spinal cord injury.
45) The pharmaceutical composition any of the preceding paragraphs, wherein the Gi-DREADD polypeptide is an optimized Gi-DREADD polypeptide.
46) The pharmaceutical composition of any of the preceding paragraphs, wherein the Gi-DREADD polypeptide comprises the sequence of SEQ ID NO: 2.
47) The pharmaceutical composition of any of the preceding paragraphs, wherein the Gi-DREADD polypeptide has at least 95% amino acid sequence identity to SEQ ID NO: 2 and retains at least 80% of the biological activity of Gi-DREADD of SEQ ID NO: 2.
48) The pharmaceutical composition of any of the preceding paragraphs, further comprising clozapine N-oxide.
49) The pharmaceutical composition of any of the preceding paragraphs, further comprising at least a second therapeutic compound.
50) A pharmaceutical composition comprising an effective amount of Kir2.1 polypeptide or a vector comprising a nucleic acid sequence encoding the Kir2.1 polypeptide and a pharmaceutically acceptable carrier, for use in treating spinal cord injury.
51) The pharmaceutical composition of any of the preceding paragraphs, wherein the Kir2.1 polypeptide comprises the sequence of SEQ ID NO: 3.
52) The pharmaceutical composition of any of the preceding paragraphs, wherein the Kir2.1 polypeptide has at least 95% amino acid sequence identity to SEQ ID NO: 3 and retains at least 80% of the biological activity of Kir2.1 of SEQ ID NO: 3.
53) The pharmaceutical composition of any of the preceding paragraphs, further comprising clozapine N-oxide.
54) The pharmaceutical composition of any of the preceding paragraphs, further comprising at least a second therapeutic compound.
55) A pharmaceutical composition comprising an effective amount of an agent of paragraphs 19-21 and a pharmaceutically acceptable carrier, for use in treating spinal cord injury.
56) The pharmaceutical composition of any of the preceding paragraphs, further comprising at least a second therapeutic compound.
57) A method for treating a spinal injury, comprising administering to a subject having a spinal injury an effective amount of CLP290.
58) The method of any of the preceding paragraphs, wherein CLP290 crosses the blood brain barrier.
59) The methods of any of the preceding paragraphs, wherein the spinal injury is a severe spinal cord injury.
60) The method of any of the preceding paragraphs, wherein the subject is human.
61) The method of any of the preceding paragraphs, wherein the subject has been diagnosed with a spinal injury.
62) The method of any of the preceding paragraphs, wherein the subject has been previously treated for a spinal injury.
63) The method of any of the preceding paragraphs, wherein prior to administering, the subject is diagnosed with having a spinal cord injury.
64) The method of any of the preceding paragraphs, wherein the subject is further administered at least a second spinal injury treatment.
65) The method of any of the preceding paragraphs, wherein the subject is further administered at least a second therapeutic compound.
66) The method of any of the preceding paragraphs, wherein the second therapeutic compound is selected from the group consisting of osteopontin, a growth factor, or 4-aminopuridine.

### Examples

### Introduction

Most human spinal cord injuries (SCIs) are anatomically incomplete, with spared axons spanning the damaged spinal segments. However, about a half of these patients have a total loss of muscle control and sensation below the injury level (Fawcett et al., 2007; Kakulas, 1999), suggesting that spared connections are functionally dormant. Remarkably, recent studies have demonstrated that epidural stimulation combined with rehabilitative training allows some chronically paralyzed patients with SCI to regain voluntary movement (Angeli et al., 2014; Harkema et al., 2011). A postulated mechanism is that these manipulations reactivate such dormant spinal circuitry, enabling brain-derived signals to be relayed to the spinal cord. However, it is largely unknown why this spared spinal circuitry is dysfunctional after SCI, and how it can best be reactivated.

In the case of hindlimb function, the spinal center for executing basic locomotion, the central pattern generator (CPG), is primarily located in the lumbar spinal cord (Frigon and Rossignol, 2008; Gerasimenko et al., 2008; Grillner and Wallen, 1985; Kiehn, 2016). Classical studies, using spinal cords isolated from neonatal animals, showed that pharmacological manipulations of neuronal excitability could initiate and modulate the efferent patterns (Cazalets et al., 1992; Cowley and Schmidt, 1995; Kiehn, 2006). In intact animals, the output of the lumbar locomotor center is controlled in part by descending commands from the brain. After being deprived of these inputs by SCI, the lumbar spinal cord fails to initiate locomotor function, even when sensory afferents are intact. In order to restore function after SCI, it is crucial to re-establish the connections between descending inputs and the lumbar spinal cord. For example, compensatory axon regrowth and synapse reorganization could enhance such connections at different spinal levels after SCI (Ballermann and Fouad, 2006; Bareyre et al., 2004; Courtine et al., 2008; Filous and Schwab, 2017; He and Jin, 2016; Jankowska and Edgley, 2006; Rosenzweig et al., 2010; Takeoka et al., 2014; van den Brand et al., 2012; Zaporozhets et al., 2011). In severe spinal cord injury in which the majority of descending spinal-projecting pathways are damaged, the engagement of intraspinal networks, consisting of local interneurons limited to single spinal segments and projecting propriospinal neurons whose axons cross many spinal segments, can function as indirect relay pathways to receive and transmit brain-derived motor commands to the lumbar spinal cord (O'Shea et al., 2017; Zaporozhets et al., 2011).

Different hypotheses have been put forward to explain why spared connections have a limited ability to compensate after SCI. For example, the firing and conduction properties of neurons with spared descending axons could be compromised (Edgerton et al., 2008; Arvanian et al., 2009; Sawada et al., 2015). Alternatively, local spinal cord circuits could be rendered nonfunctional by injury, such that they may no longer be able to relay or integrate the spared descending inputs (Courtine et al., 2008; Edgerton et al., 2008; Rossignol and Frigon, 2011). The contribution of these and other factors remains to be characterized. Moreover, it is not even clear whether inhibiting or enhancing the excitability of spared spinal neurons would be beneficial for functional recovery after SCI.

Remarkable progress has been made in characterizing the cellular and molecular mechanisms regulating neuronal excitability. As a result, a number of small molecule compounds have been developed to target key regulators, such as ion channels and receptors, and their pharmacological properties have been well characterized. Importantly, many of these compounds can efficiently cross the blood-brain-barrier (BBB), which enables the systemic administration of these small molecules to analyze their effects in SCI animal models. Thus, presented herein is a non-biased compound screening approach to identify neuronal activity modulators that can reactivate dormant spinal circuitry, and ultimately mediate functional recovery, in SCI models.

### Results

CLP290 Restores Consistent Stepping Ability in Paralyzed Mice with Staggered Lesions.

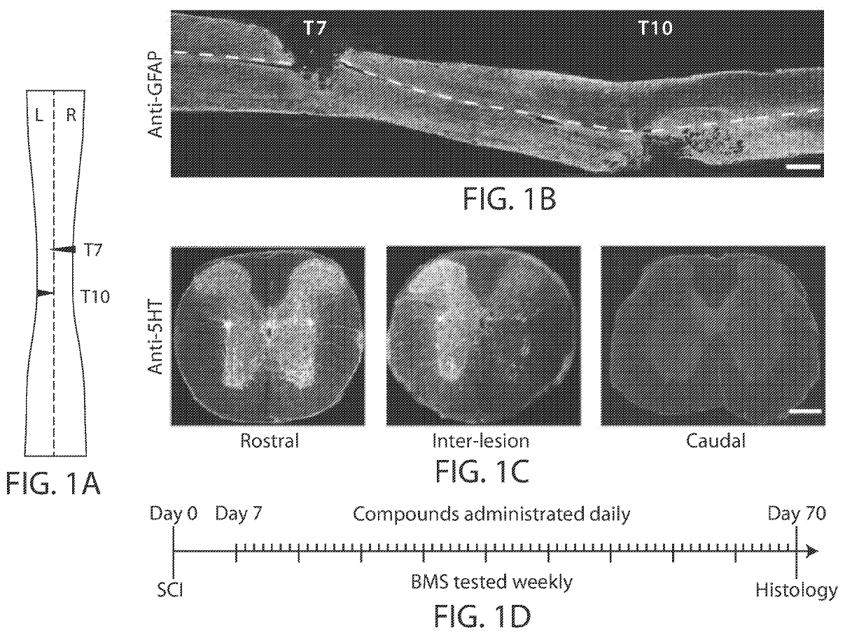

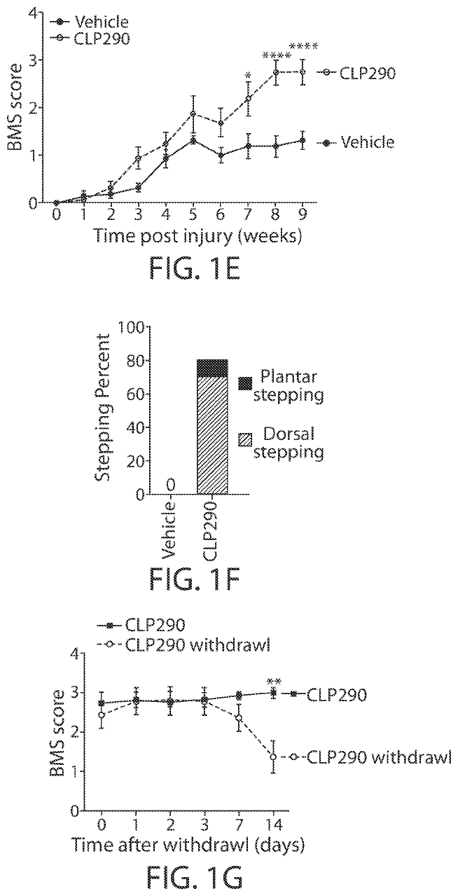

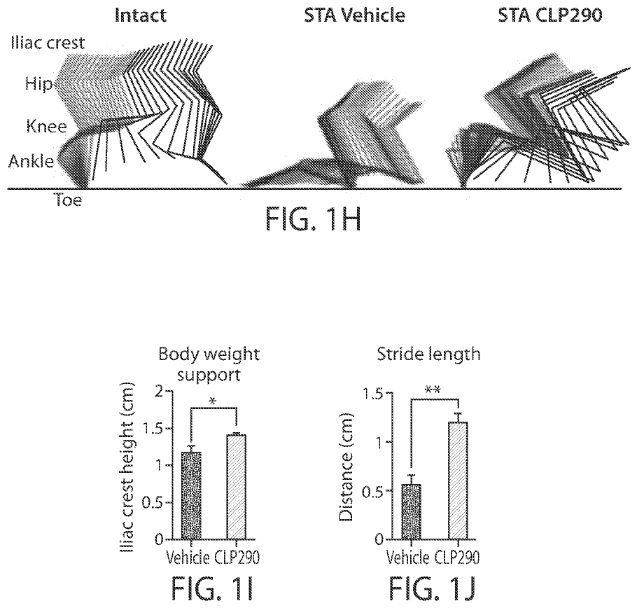

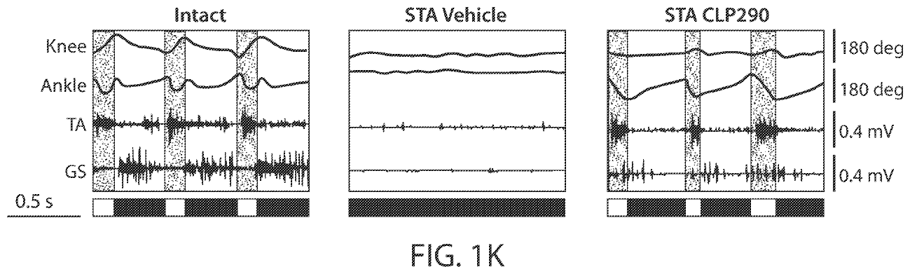

A staggered lesion paradigm was optimized in which two lateral hemisections were performed at the thoracic (T) 7 and T10 levels simultaneously (FIGS. 1A and 1B), similar to the model previously described (Courtine 2008; van den Brand, 2012). The T10 lesion is a lateral hemisection that ends at the spinal cord midline, while the T7 lesion, contralateral to the T10 lesion, extends slightly beyond the midline (FIG. 1A). With this double hemisection procedure, all descending axons passing T10 are severed, leaving only those crossing the midline between T7 and T10 intact (FIG. 1C). Indeed, by immunohistochemistry with anti-5-HT antibodies, which label serotonergic axons, descending serotonergic axons could be detected in the spinal cord segments between the lesions, but not in the lumbar spinal cord (FIG. 1C). Thus, a relay zone remains between and around the lesions (T7 and T10) where descending axons terminate, and where some propriospinal neurons maintain their connections with lumbar spinal neurons (see herein below).

The mice with this staggered lesion exhibited nearly complete and permanent hindlimb paralysis (FIGS. 1E and 1F). During the 10 weeks after injury, injured mice rarely showed ankle movement and never displayed any type of stepping, with a score of 0.5 or 1 on the Basso Mouse Scale (BMS), an established open field locomotion test (Basso et al., 2006). Thus, the spared relay pathways between T7 and T10 must remain dormant.

This double hemisection SCI model was used to seek small molecule compounds that could reactivate the spared, but dormant, spinal connections by monitoring hindlimb motor performance during over-ground locomotion. To this end, daily compound treatment was started 1 week after injury and then monitored the BMS scores approximately 24 hours after the previous day's compound treatment on a weekly basis (FIG. 1D). Behavioral outcomes observed at these time points likely reflect sustained effects of the treatment, which are more clinically relevant.

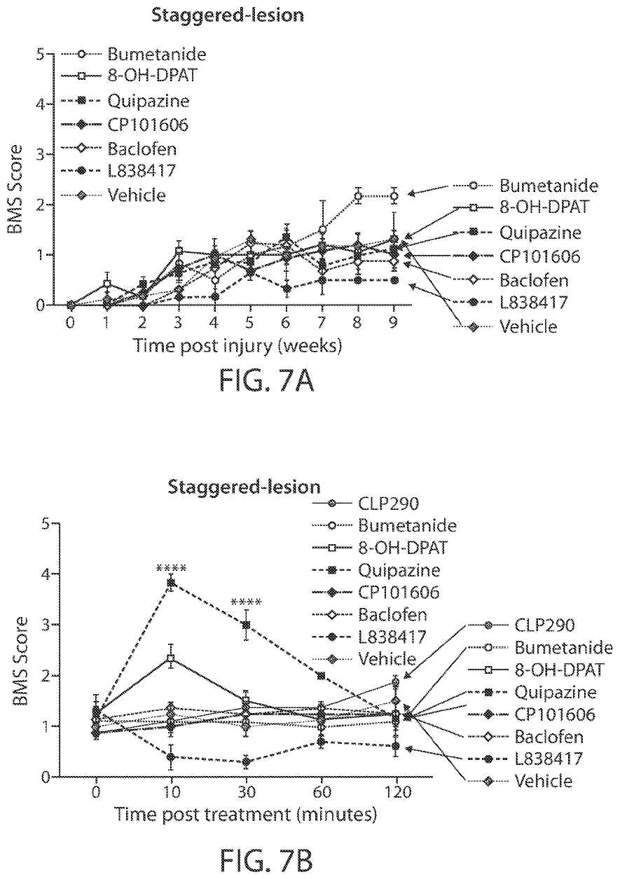

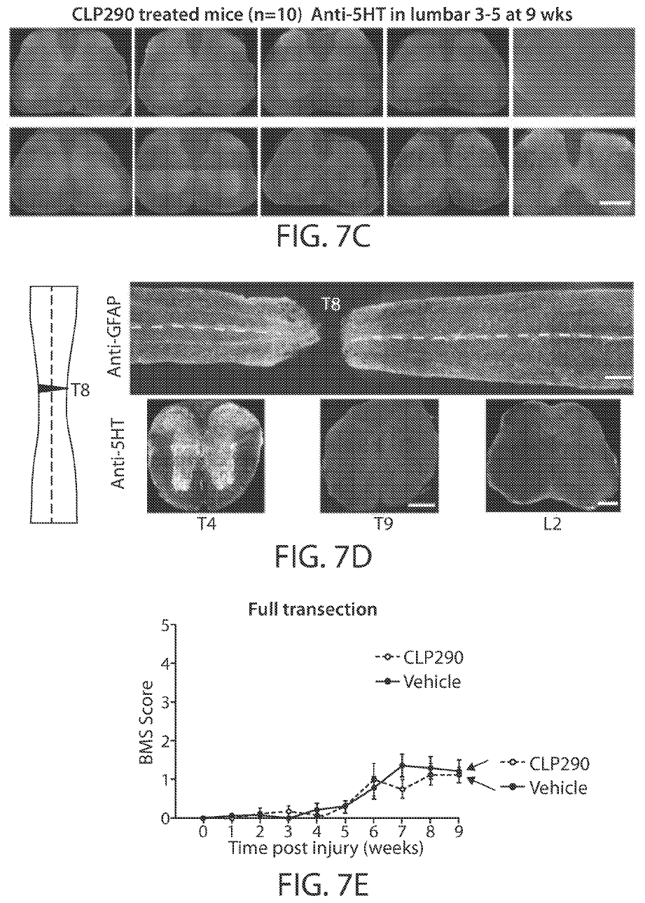

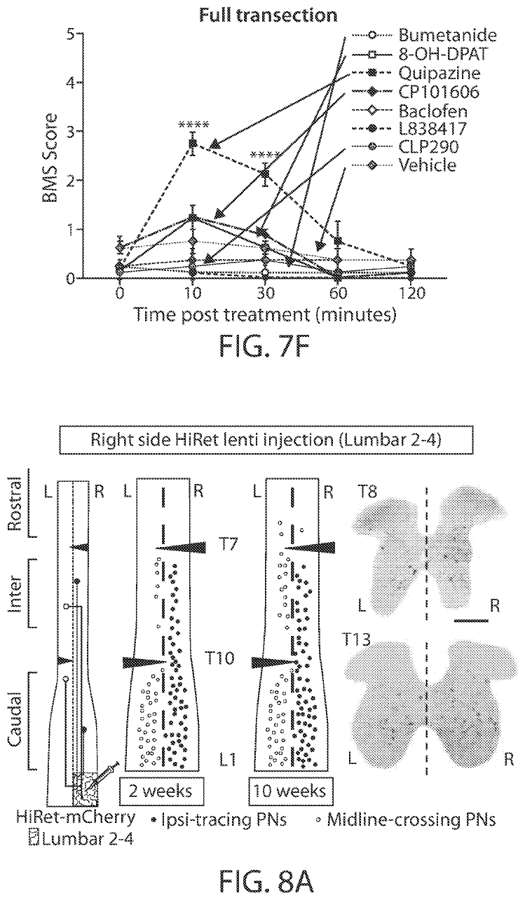

Candidate compounds were chosen based on their ability to modulate neuronal excitability upon systemic delivery. They included: baclofen, a GABA receptor agonist; bumetanide, an inhibitor of the Na⁺/2Cl⁻/K⁺ co-transporter (NKCC); CLP290, an agonist of the neuron-specific K⁺—Cl⁻ co-transporter (KCC2), also called SLC12A5; L838,417, a GABAA positive allosteric modulator; CP101606, an NMDA treceptor antagonist; 8-OHDPAT, a 5HT1A/7 agonist; and quipazine, a 5HT2A/C agonist (FIGS. 1E, 7A). One of these treatments resulted in significant improvements in stepping ability within the first 2-3 weeks after daily treatment. However, in CLP290-treated mice, functional recovery first appeared by 4-5 weeks, and became significant from 7 weeks after treatment (FIG. 1E). Bumetanide also showed some effects, but without statistical significance (FIG. 7A). Thus, further analyses focused on CLP290-treated SCI mice.

The majority (80%) of CLP290-treated mice recovered consistent hindpaw plantar placement, and weight-bearing stepping (most with dorsal stepping and some with plantar stepping; FIG. 1F), in contrast to control mice and mice treated with other compounds, which predominantly demonstrated paralyzed hindlimbs. This extent of recovery is functionally significant, as stepping ability has been implicated as the limiting step for functional recovery in severe injury models (Schucht et al., 2002). During stepping, CLP290-treated mice could partially support their body weight, and exhibited significantly increased oscillation of hindlimb joints (FIGS. 1H-1K). By electromyogram (EMG) recording in control injured mice (FIG. 1K), it was found that the ankle flexor tibialis anterior muscle (TA) was rarely active, while activity of the extensor gastrocnemius soleus muscle (GS) was never observed. In contrast, CLP290-treated mice showed both TA and GS activity (FIG. 1K). Consequently, the total hindlimb stride length in CLP290 treated mice was significantly increased (FIG. 1J). Intriguingly, different from intact mice which have alternating activation of TA (swing phase) and GS (stance phase) during stepping gait, CLP290-treated SCI mice showed co-activation of TA and GS during the swing phase (FIG. 1K), a sign of suboptimal bodyweight support.

Further, in mice with CLP290-induced recovery, the BMS scores remained significantly higher than controls for 1-2 weeks after stopping treatment (FIG. 1G), suggesting that sustained functional recovery resulted from CLP290 treatment. At the end of these experiments, no immunostaining with the anti-5-HT antibody was observed in the lumbar region, and verified the success of staggered lesions in these mice (FIG. 7C). Together, these results demonstrate that CLP290 treatment enables most paralyzed mice to restore weight-bearing stepping capacity in a sustained fashion.

CLP290 Treatment does not Induce Functional Improvement in Mice with a Complete Lesion.

CLP290's effects could result from reactivating the spared dormant descending connections in the spinal cord after SCI. However, it could also act directly on the lumbar spinal cord, independently of descending inputs. To distinguish between these possibilities, the same CLP290 treatment were applied to mice with a complete T8 spinal cord transection, in which no axons cross the lesion site (FIG. 7D), and found that CLP290 failed to promote any significant functional recovery (FIG. 7E). Conversely, the 5-HT receptor agonist quipazine led to a rapid, but transient, BMS improvement (starting at 10 mins and lasting for less than 2 hours) in both the staggered lesion (FIG. 7B) and T8 complete transection models (FIG. 7F). Therefore, different from this transient effector that acts directly on the lumbar spinal cord, the effects of CLP290 on functional improvement are dependent on spared connections.

CLP290 does not Impact Axon Regrowth.

As mice with either staggered lesions or complete lesions display similar SCI-associated behavioral deficits (pain and spasticity), results presented herein show that CLP290 induces functional recovery in mice with staggered lesions only suggest that the functional improvements of CLP290 are likely independent of such analgesic and anti-spastic effects. Thus, the possible mechanisms for CLP290 are likely to rely on the spared relay pathway, for example by promoting axonal sprouting, and/or by increasing the fidelity of the relay pathway signal, to the lumbar spinal cord.

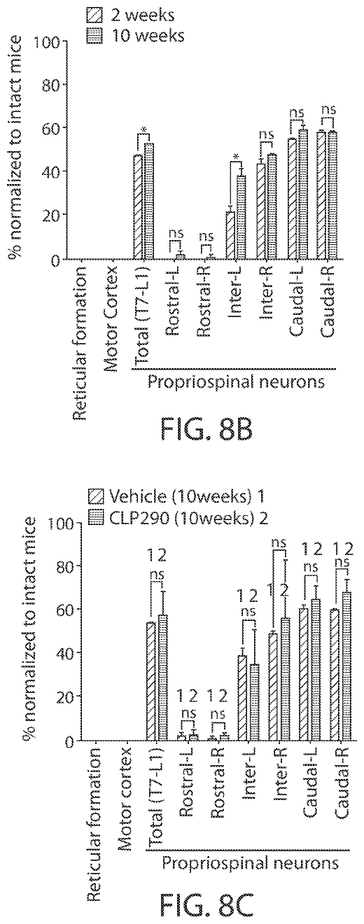

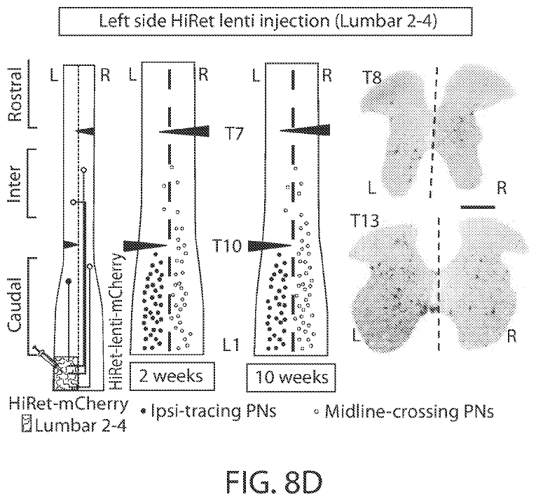

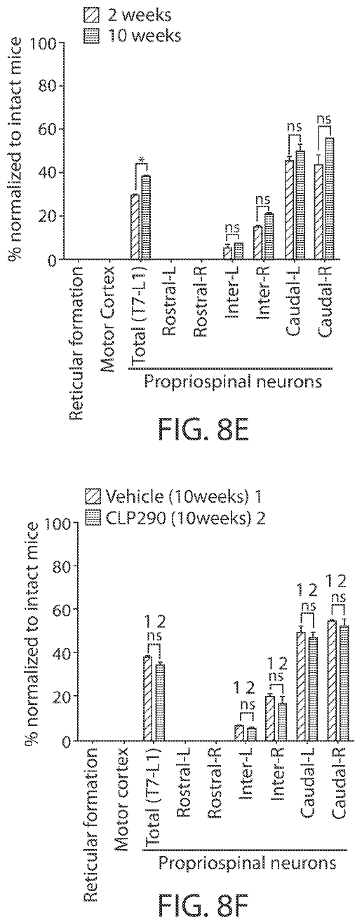

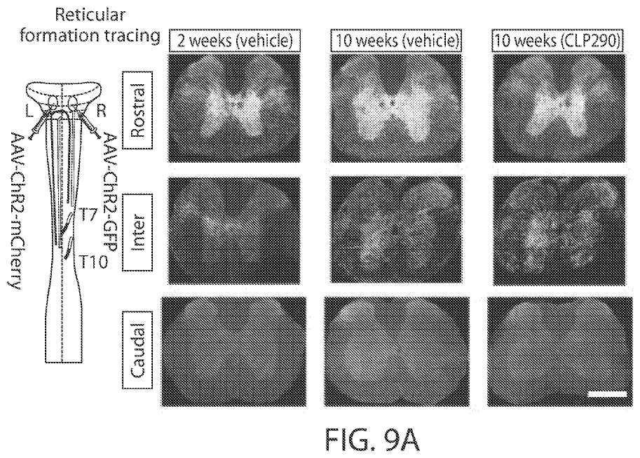

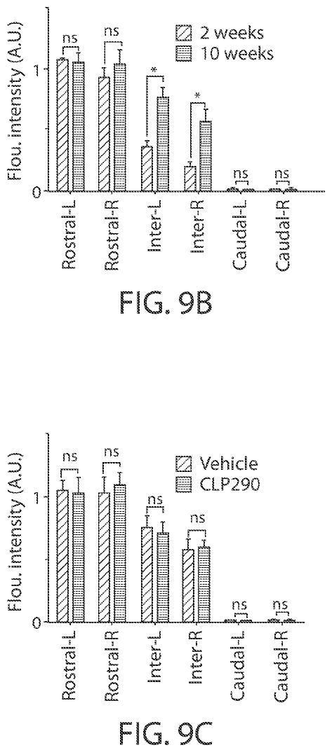

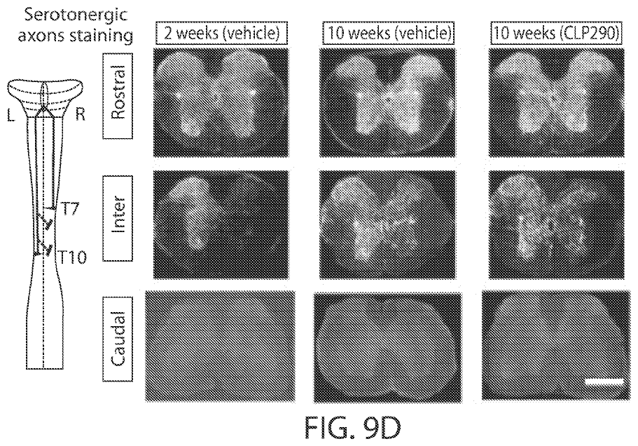

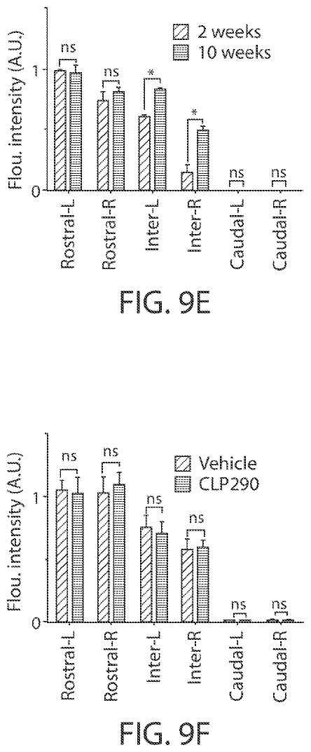

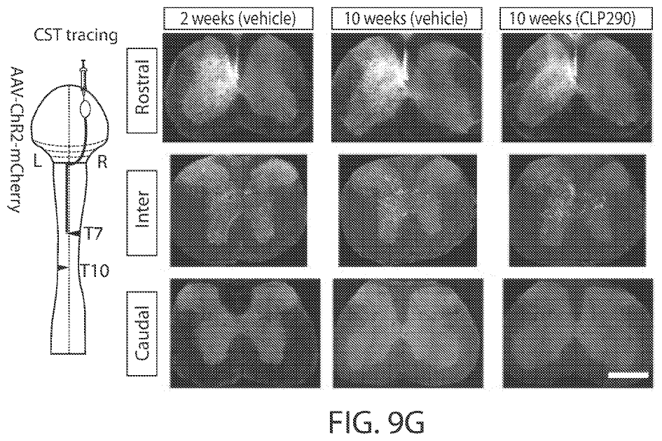

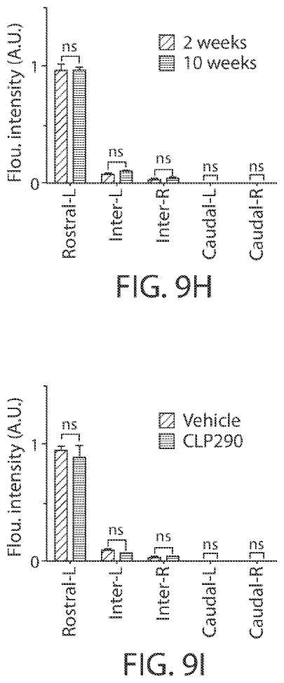

To test these possibilities, it was determined whether CLP290 increased the regrowth of spared propriospinal axons, and/or their connecting axons from the brain. To analyze neuronal projections to the hindlimb locomotor control center in each condition, a retrograde tracing pseudotyped lentiviral vector (HiRet) expressing mCherry (HiRet-mCherry) (Kato et al., 2011; Wang et al., 2017; Liu et al., 2017) was injected into the lumbar enlargement (L2-L4). At 2 weeks after injury, most retrogradely labeled neurons were found in the spinal cord segments between and around the lesions, with few above the lesion and none in the brain (FIG. 82). The number of retrogradely traced neurons in the spinal cord increased by 10 weeks after injury, consistent with previous reports (Courtine et al., 2008), but CLP290 treatment did not affect these measures (FIGS. 8C and 8F). Similarly, anterograde tracing from the brain with AAV-ChR2-mCherry and AAV-ChR2-GFP, failed to reveal increased sprouting of descending brainstem reticulospinal axons (FIG. 9A-9C), or corticospinal axons (FIG. 9G-9I), in the spinal cords of CLP290-treated mice at 2 and 10 weeks after injury. Similarly, the sprouting of serotonergic axons detected by 5-HT immunohistochemistry was also not affected by CLP290 treatment (FIG. 9D-9F). Thus, it is unlikely that CLP290 acts by promoting the regrowth of brain-derived descending axons into the relay zone, or propriospinal axons projecting to the lumbar spinal cord.

KCC2 Expression Mimics the Effects of CLP290 to Promote Functional Recovery.

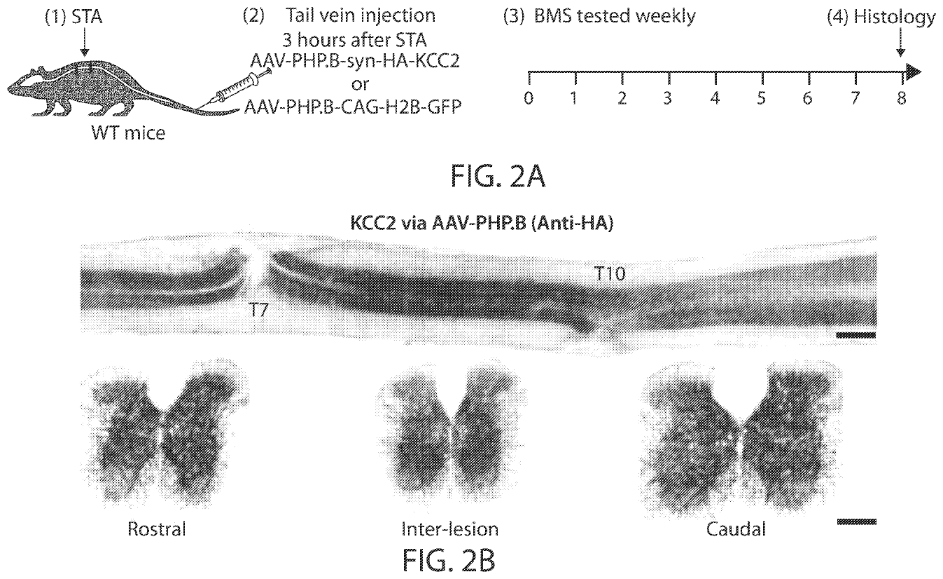

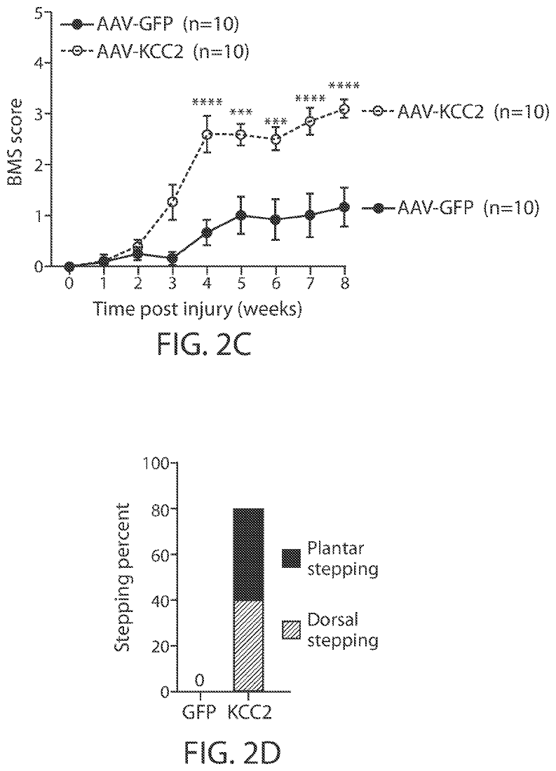

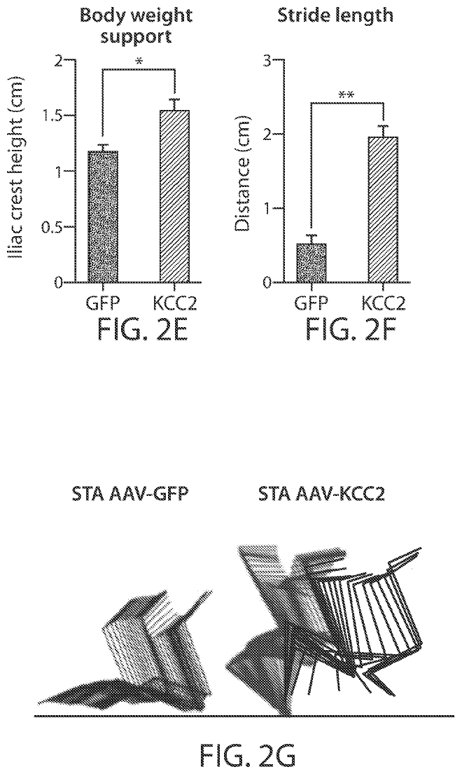

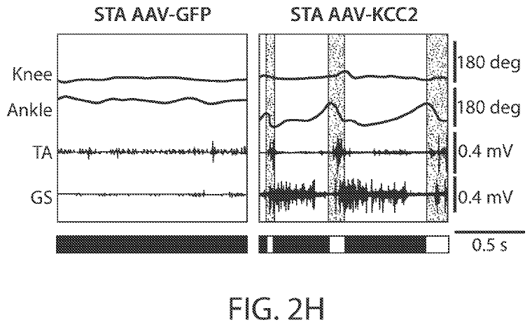

CLP290 was identified as an activator of the K⁺—Cl⁻ co-transporter KCC2, but it may also act on other targets (Gagnon et al., 2013). Thus, it was determined whether overexpression of KCC2 in CNS neurons had effects similar to CLP290 in staggered-lesioned mice. Taking advantage of AAV-PHP.B vectors that can cross the BBB in adult mice (Deverman et al., 2016), AAV-PHP.B expressing KCC2 under control of the human synapsin promoter (AAV-PHP.B-syn-HA-KCC2) was injected into the tail vein. Injections were performed directly after injury because KCC2 took 1-2 weeks to be detectably expressed. Weekly behavioral monitoring were then performed (FIG. 2A). As shown in FIG. 2B, AAV-PHP.B-KCC2 treatment resulted in widespread expression of HA-tagged KCC2 in all spinal cord segments as analyzed 8 weeks post injury. In contrast to control AAV-PHP.B-H2B-GFP, AAV-PHP.B-KCC2 treatment led to significant functional recovery (FIG. 2C-2H), to an extent similar to, or greater than, CLP290 (FIG. 1E-1J). Indeed, at 8 weeks after AAV-KCC2 treatment, 80% of these mice were able to step with ankle joint movement involving TA and GS, and about a half of these mice could achieve plantar stepping with both ankle and knee movements (FIGS. 2D and 2H). Furthermore, AAV-KCC2 treated mice could partially support their body weight with frequent GS firing during the stance phase (FIGS. 2E and H).

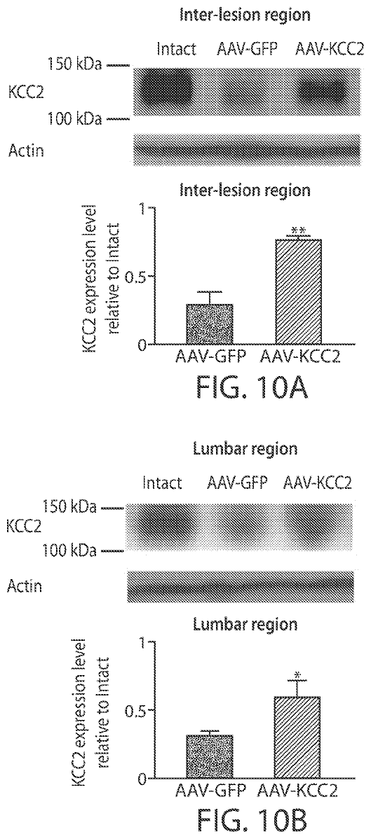

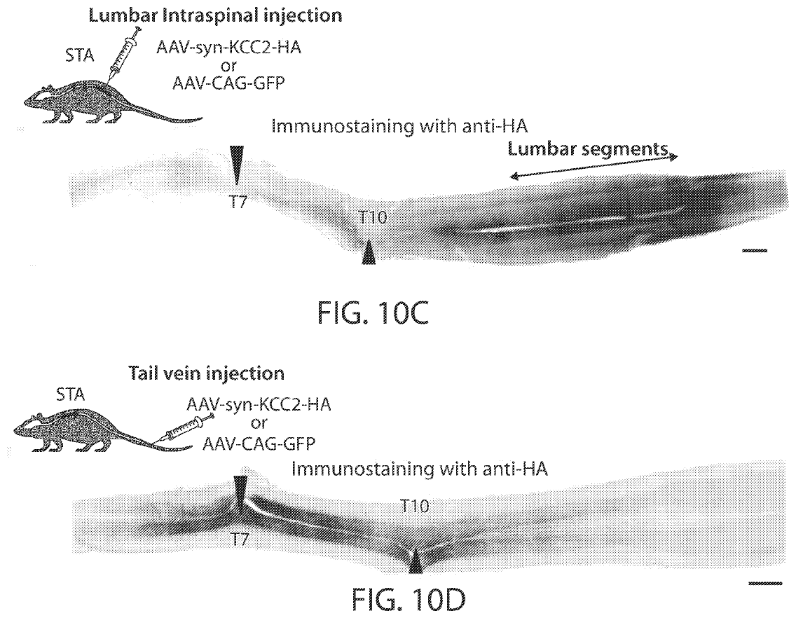

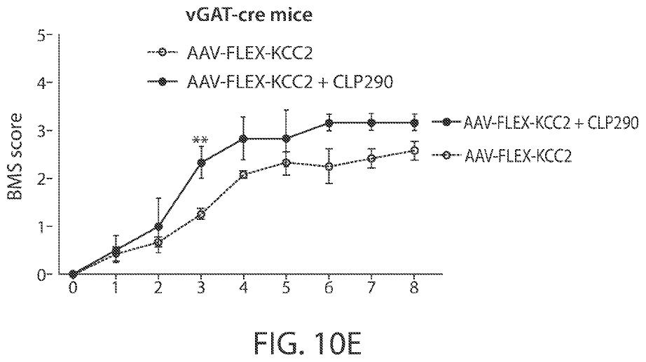

At the termination of this experiment (9-10 weeks after injury), the expression levels of KCC2 in the spinal cord was analyzed by Western blotting. In control mice, KCC2 is significantly reduced in the lumbar and inter-lesion spinal cord segments after injury (FIGS. 10A and 10B), consistent with previous reports (Boulenguez et al., 2010; Cote et al., 2014). However, AAV-KCC2 treatment restored KCC2 expression to levels significantly closer to uninjured mice relative to AAV-GFP controls (FIGS. 10A and 10B). Thus, AAV-KCC2 likely acts by counteracting SCI-induced KCC2 down-regulation.

Selective KCC2 Expression in Inhibitory Interneurons Leads to Functional Recovery.

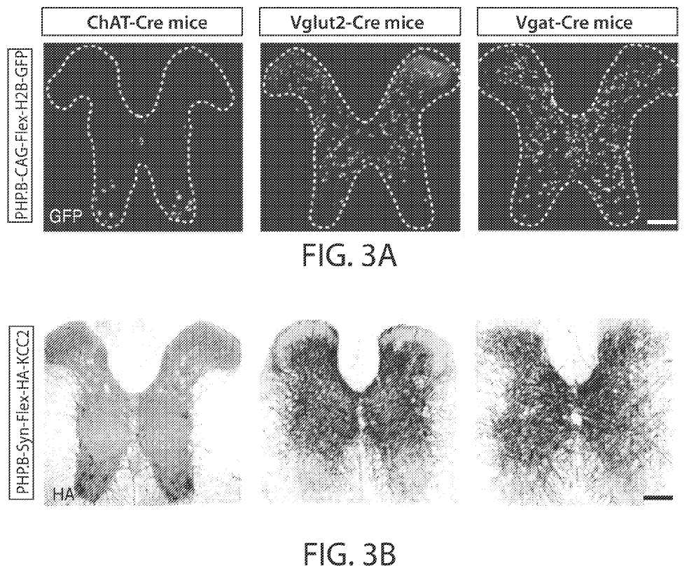

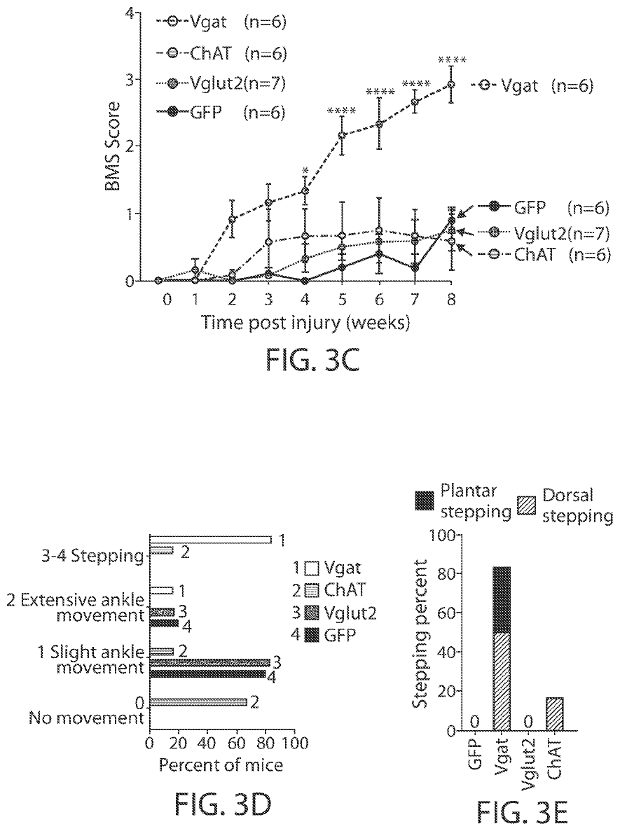

It was next assessed whether KCC2 expression in specific types of neurons accounts for the observed functional recovery. To do this, AAV-PHP.B-FLEX-KCC2 (Cre-dependent KCC2 expression) was injected into the tail vein of adult mice of Vglut2-Cre (for excitatory neurons (Tong et al., 2007)), Vgat-Cre (for inhibitory neurons (Vong et al., 2011)) or Chat-Cre (for motor neurons and a subset of interneurons (Rossi et al., 2011)) directly after injury (FIGS. 3A and 3B). In contrast to Chat-Cre and Vglut2-Cre mice, Vgat-Cre mice injected with AAV-PHP.B-FLEX-KCC2 showed significant functional recovery (FIGS. 3C-3E), to an extent similar to CLP290 treatment (FIG. 1), or non-selective KCC2 expression (FIG. 2). Thus, these results suggest that KCC2 dysfunction or down-regulation in inhibitory interneurons limits hindlimb functional recovery in staggered-lesioned mice.

KCC2 Acts Through Inhibitory Interneurons in the Spinal Cord Segments Between and Around the Staggered Lesions to Induce Functional Recovery.

As shown in FIGS. 7 and 8, propriospinal neurons in the relay zone, consisting of the spinal cord segments between and below the staggered lesions, are likely to relay the brain-derived signals to the lumbar spinal cord. Thus, there are two possible mechanisms for KCC2-mediated hindlimb functional recovery in stagger-lesioned mice: (1) KCC2 acts on the inhibitory interneurons in the lumbar segments (L2-5) to facilitate the integration of propriospinal inputs; and/or (2) KCC2 acts on the inhibitory neurons in the relay zone above the lumbar spinal cord to facilitate the integration of brain-derived inputs from descending pathways, and/or its relay to the lumbar spinal cord.

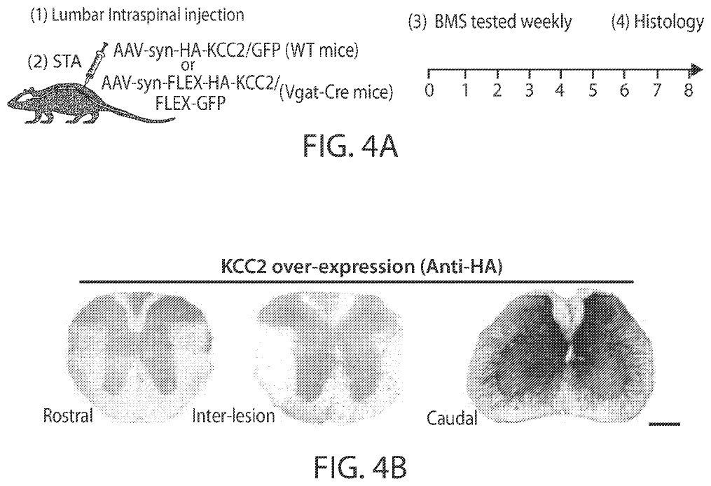

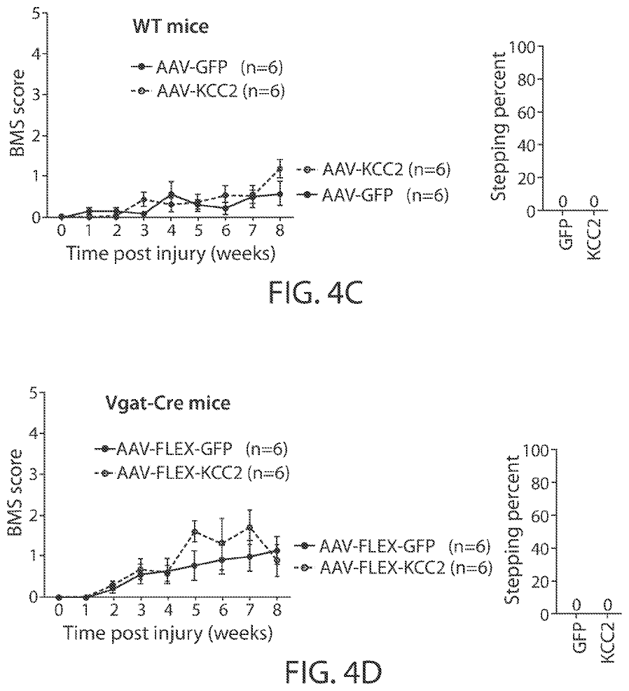

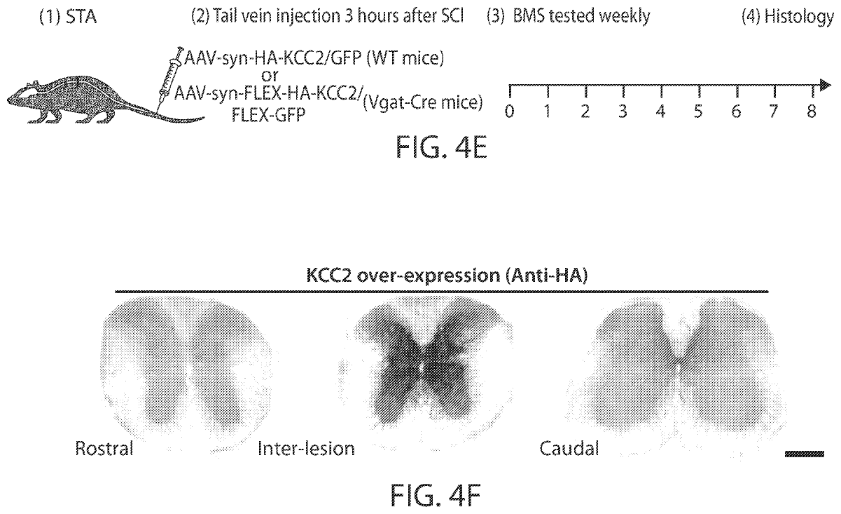

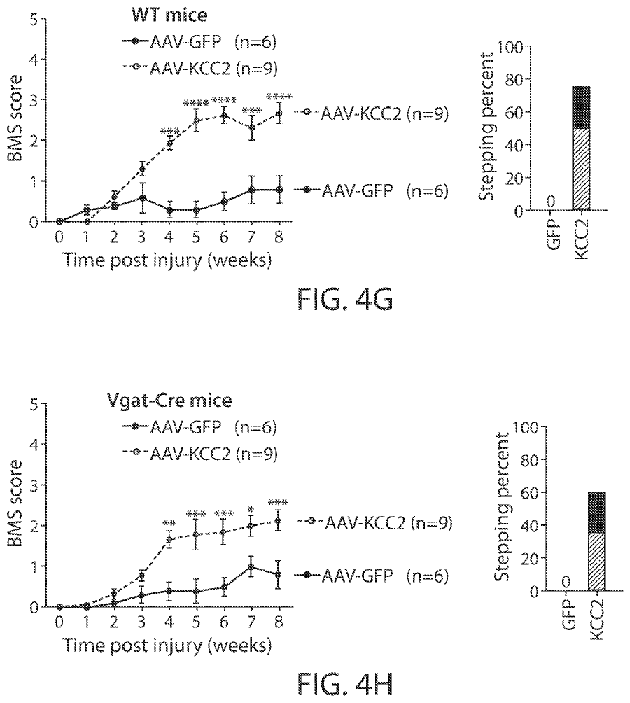

To test these possibilities, AAV-KCC2 or AAV-FLEX-KCC2 were injected locally into lumbar segments (L2-5) of wild type mice or Vgat-Cre mice (FIGS. 4A-B and 10C). These treatments did not lead to significant functional recovery (FIGS. 4C-D), suggesting that the inhibitory neurons in the lumbar spinal cord are unlikely to mediate the functional recovery effects of KCC2.

To introduce KCC2 into spinal cord segments between and around the staggered lesions, the compromised blood-spinal cord-barrier around the lesion sites acutely after the injury were taken advantage of AAV-KCC2 or AAV-FLEX-KCC2 were injected into the tail vein of wild type or Vgat-Cre mice, respectively, at 3 hours after over-staggered lesions (FIG. 4E). As a result, KCC2 expression spanned between T5 and T12 (FIGS. 4F and 10D). In these animals, a significant and persistent functional recovery, with increased BMS performance, was observed in both groups of mice (FIGS. 4G and 4H), to extents comparable to AAV-PHP.B-KCC2 treatment (FIG. 2). In these Vgat-Cre mice with AAV-FLEX-KCC2, accompanying CLP290 treatment did not significantly enhance functional recovery at most time points (FIG. 10E), consistent with the notion that the effects of CLP290 were mainly mediated by activating KCC2 in these inhibitory interneurons. Thus, KCC2/CLP290 primarily acts through inhibitory neurons in the relay zone, between and adjacent to the lesion sites in thoracic spinal cord levels, to facilitate hindlimb functional recovery.

CLP290/KCC2 Alters Excitability and Relay Formation.

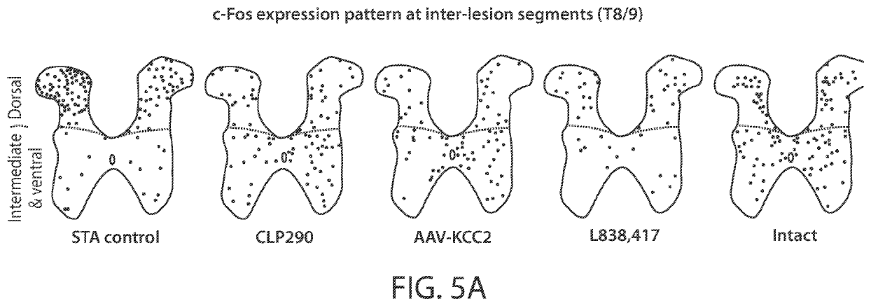

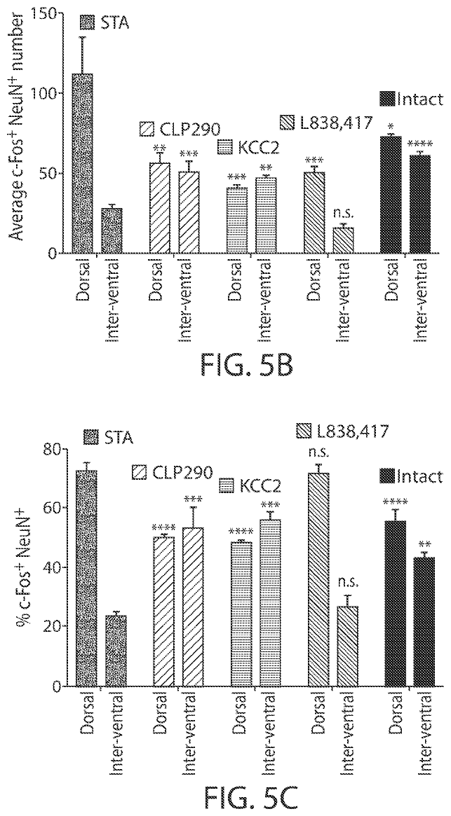

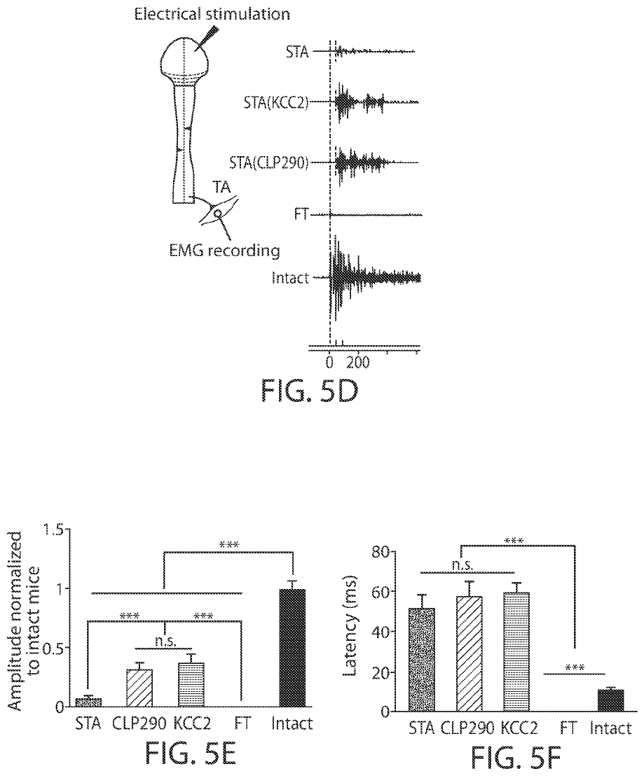

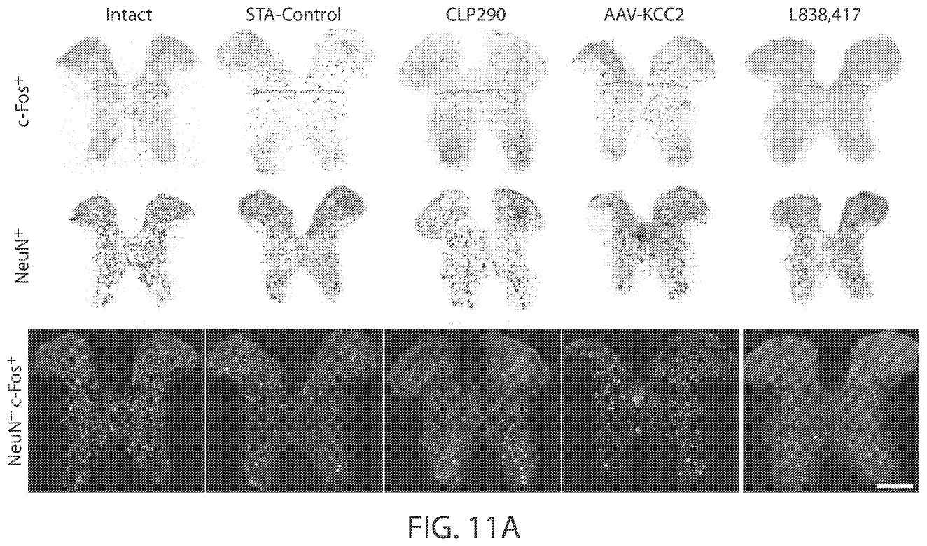

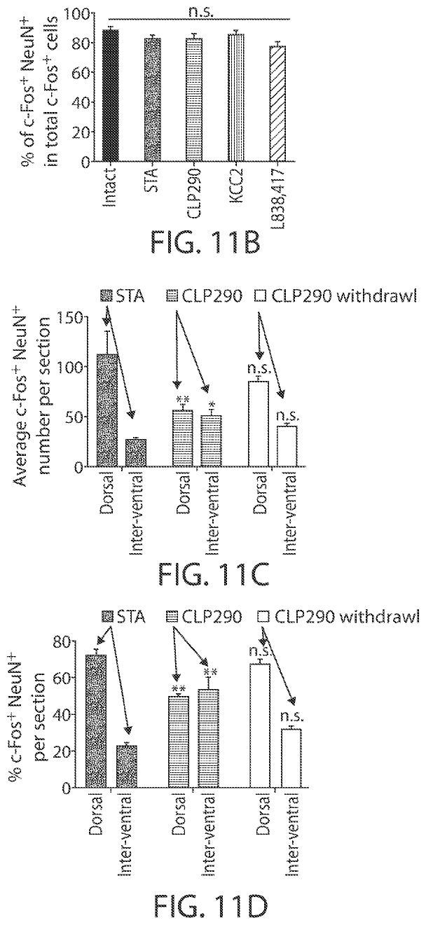

In mature neurons, GABA and glycine are inhibitory because they open chloride channels, which allow chloride ion influx leading to hyperpolarization. In contrast, during development, the elevated intracellular chloride levels render GABAA- and glycine-mediated currents depolarizing and generally excitatory. During early postnatal life, KCC2 upregulation in postnatal neurons is crucial for reducing intracellular chloride concentrations, transforming excitation into inhibition (Ben-Ari et al., 2012; Kaila et al., 2014). Thus, injury-induced KCC2 down-regulation (Boulenguez et al., 2010; Cote et al., 2014) would be expected to restore an immature state in which GABA and glycine receptors can depolarize neurons. In this scenario, KCC2 activation in spinal inhibitory neurons would transform local circuits in the relay zone towards a more physiological state, which is more receptive to descending inputs. To examine this, c-Fos immunoreactivity was used as a proxy of neuronal activity in the spinal cord segments between T7 and T10 at 8 weeks after injury, and after walking on a treadmill for 1 hour. In each group, the majority of c-Fos-positive cells in these spinal segments were also positively stained with NeuN, a neuronal marker (FIG. 11A, 11B). Representative composites of c-Fos/NeuN double-positive cells are shown in FIG. 5A. In injured mice without treatment, the c-Fos-positive neurons were concentrated in the dorsal horn of the spinal cord (FIG. 5A-5C), perhaps reflecting hypersensitivity to peripheral sensory inputs in these injured mice. With CLP290 or AAV-KCC2 treatment, the distribution of c-Fos-positive neurons became very different, with a reduction in the dorsal horn (laminae I-V), and a significant increase in the intermediate/ventral spinal cord (FIG. 5A-5C). This KCC2-transformed distribution pattern is similar to what was detected in intact mice, in response to walking (FIG. 5A-5C). 2 weeks after withdrawal of CLP290 treatment, the c-Fos pattern returned to what seen without treatment (FIGS. 11C and 11D), consistent with the behavioral outcomes (FIG. 1G). Taken together, these findings suggest that increasing KCC2 activity restores a more physiological neuronal activity pattern to the local spinal cord circuitry.

As a control, c-Fos immunoreactivity was examined in the spinal cord of staggered injured mice following chronic treatment with L838,417, a GABA agonist which has been shown to reduce neuropathic pain (Knabl et al., 2008). As shown in FIG. 5A-5B, L838,417 reduced c-Fos-positive neurons in dorsal horn, but without increasing those in intermediate zones and ventral region, corroborating the results that L838,417 treatment failed to promote functional motor recovery (FIG. 7A). As the intermediate and ventral spinal cord are major termination zones of descending inputs, increased neuronal activity in this area after CLP290/KCC2, but not L838,417, treatment likely reflects improved responses to descending inputs. Thus, these results suggest that chronic KCC2/CLP290 treatment transform the SCI-induced, sensory-centralized activation pattern of the relay zone, into a state under control of both sensory and descending pathways.

To test directly if the treated spinal cord could more efficiently relay descending inputs to the lumbar spinal cord, cortical stimulation was performed and recorded EMG responses in the TA muscle (FIG. 5D). The latency of the cortical-stimulating response was significantly delayed in SCI mice compared to intact mice, and KCC2-related treatments failed to shorten the latency of the stimulation response (FIGS. 5D and 5E). These results are consistent with the notion that multiple synaptic connections exist in the KCC2-activated circuitry, which relays cortical stimulation to the motor neurons in the lumbar cord of injured mice. On the other hand, the amplitude of evoked EMG signals was significantly increased in injured mice with AAV-PHP.B-syn-HA-KCC2 or CLP290 treatment, compared to controls (FIGS. 5D and 5F), suggesting that KCC2 enhanced the relay efficiency of this spinal circuitry. Thus, KCC2 treatment facilitates the transmission of descending inputs from the brain to the lumbar spinal cord.

DREADD-Assisted Modulation of Inhibitory Neuron Excitability Mimics the Effects of KCC2/CLP290.

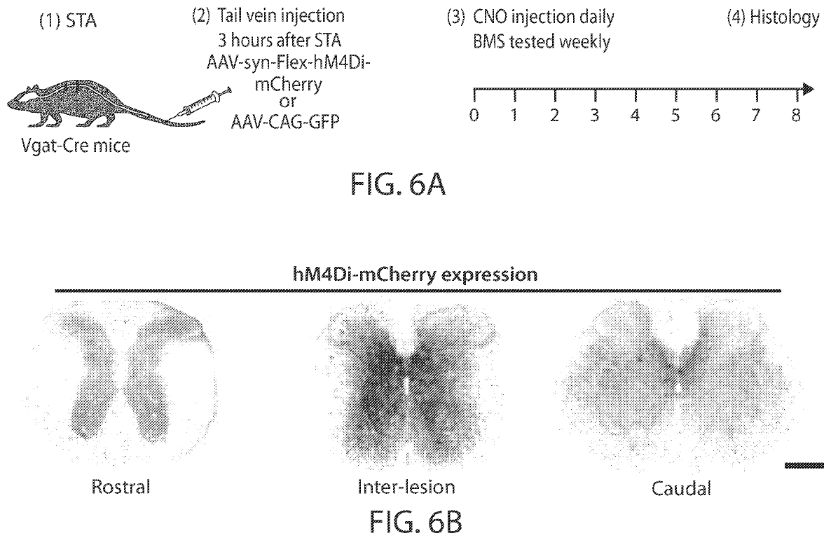

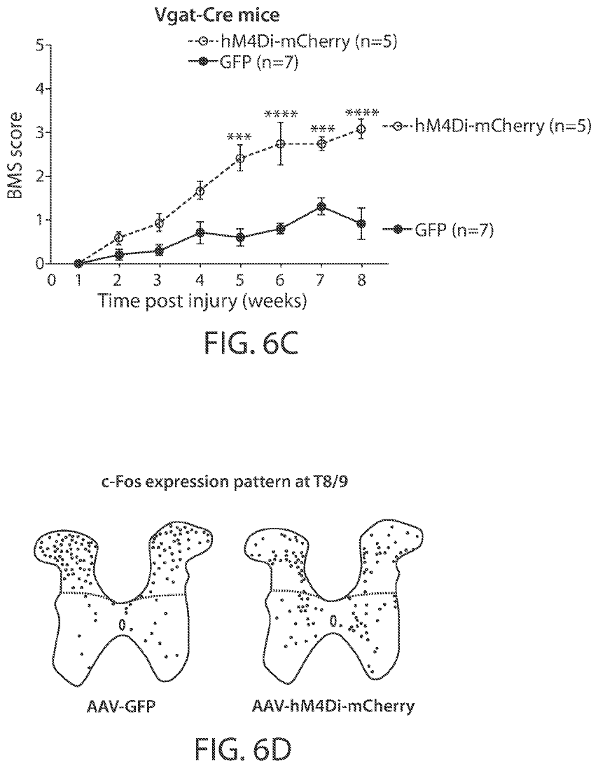

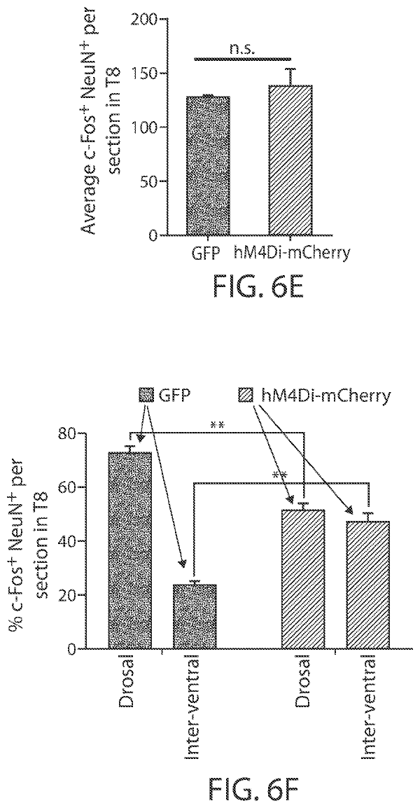

To test if reducing the excitability of inhibitory interneurons could mimic the effects of KCC2 and CPL290, hM4Di-mCherry was expressed, an inhibitory Gi-coupled receptor Gi-DREADD (Krashes et al., 2011), in inhibitory interneurons between and the around lesion by injecting AAV9 vectors (AAV9-FLEX-hM4Di-mCherry or AAV9-GFP) into the tail vein of Vgat-Cre mice 3 hours after injury (FIG. 6A). Clozapine N-oxide (CNO), which selectively activates Gi-DREADD (Roth, 2017), wase administered daily and monitored behavior weekly. When tested at 24 hours after CNO administration (using the same treatment schedule as for CLP290), it was found that injured mice with hM4Di, but not GFP, showed a similar degree of sustained functional recovery as observed with CLP290 or KCC2 treatment (FIG. 6C). Furthermore, hM4Di- and CNO-treated mice exhibited c-Fos expression patterns similar to that observed with KCC2-related treatments after continuous walking (FIG. 6D-F and FIG. 5A). Thus, these results verified the beneficial effects of reducing the excitability of inhibitory interneurons.

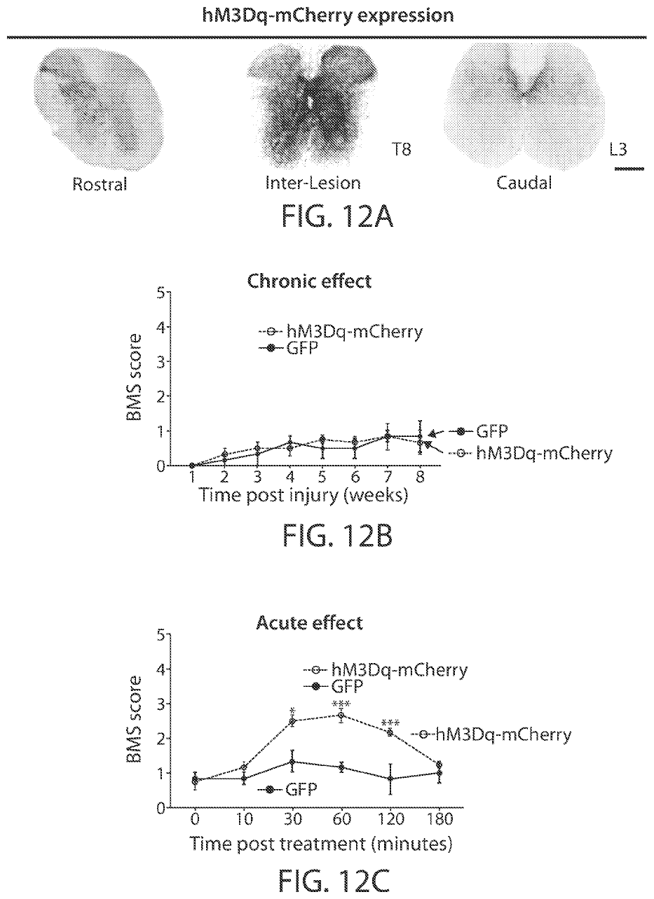

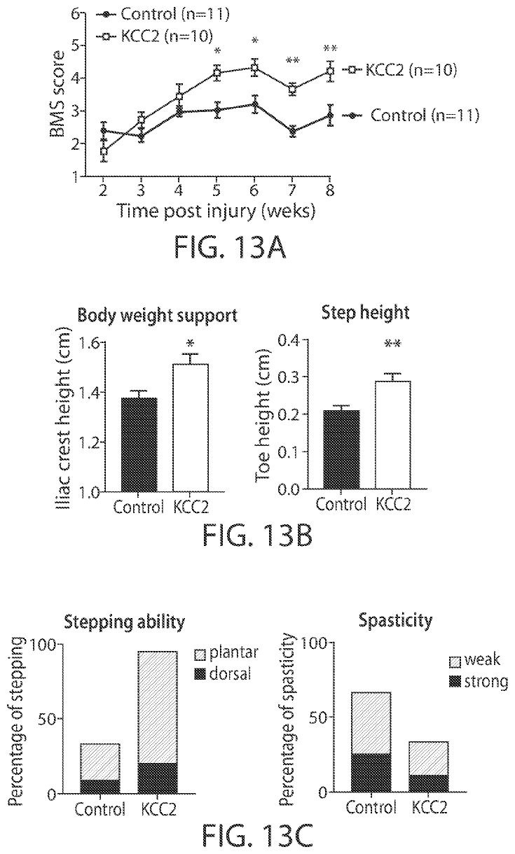

Considering that overall disinhibition within the inter-lesion segments of SCI mice via hM4Di, and the KCC2-related treatments, could increase the activity of excitatory neurons, it was asked if direct activation of excitatory interneurons could mimic the effects of inhibiting inhibitory interneurons. AAV9-GFP or AAV9-FLEX-hM3Dq-mCherry were injected to the tail vein of Vglut2-Cre mice right after staggered lesions (FIG. 12A). As shown in FIG. 12B, expression of this depolarizing hM3Dq in excitatory spinal neurons (AAV9-FLEX-hM3Dq-mCherry into Vglut2-Cre), combined with daily CNO delivery, failed to illicit functional recovery within 8 weeks of daily CNO treatment. Intriguingly, immediately after CNO administration, there was a transient functional improvement but with hindlimb spasticity (FIG. 12C, data not shown), which is similar to what was seen after quipazine treatment (FIG. 7B). Thus, directly reducing the excitability of inhibitory interneurons, but not directly increasing the excitability of excitatory interneurons, in the spinal cord is a powerful strategy to enhance responsiveness to descending inputs, and to ultimately promote lasting functional recovery after severe SCI.

### Discussion

Using a bilateral hemisection model removing all supraspinal descending connections to the lumbosacral spinal cord, it was demonstrated that chronic KCC2 activation, either pharmacologically or through AAV-assisted gene delivery, reactivates dormant spared circuitry and results in persistent hindlimb stepping. Inhibitory interneurons in the spinal cord segments between the lesions and above the lumbar spinal cord primarily mediate this effect. It is proposed that by counteracting injury-induced KCC2 downregulation, these treatments modulate neuronal excitability in the relay zone, reanimating spinal circuits that had been rendered nonfunctional by injury. As a result, these local circuits are better able to relay commands from descending projections to the lumbar spinal cord, resulting in improved behavioral recovery.

Mechanistic differences and relevance to other treatments. Previous studies showed that even in complete thoracic SCI, pharmacological approaches, such as serotonergic and dopaminergic agonists and antagonists of GABA/glycine receptors, can induce immediate, but transient, hindlimb locomotion (Courtine et al., 2009; de Leon et al., 1999; Edgerton et al., 2008; Robinson and Goldberger, 1986; Rossignol and Barbeau, 1993). Because the lumbar spinal cord is completely disconnected from the brain in these “spinal animals”, such pharmacological treatments likely act by altering the excitability of the spinal circuitry, enabling it to respond to only sensory inputs. Consistently, it was found that serotonergic agonists induced acute, but only transient locomotion (for up to 2-3 hours after compound administration), with no sustained improvements in both complete and staggered lesion models. In contrast, CLP290 induced sustained functional recovery in mice with staggered but not complete lesion. Thus, while serotonergic modulators likely act on local sensory-driven circuits in the lumbar spinal cord, CLP290 recruits dormant spared connections from the brain after SCI.

In addition, the combinatorial treatment of epidural stimulation and rehabilitation has also been shown to induce some degree of voluntary movement in rats with staggered lesions (together with a pharmacological cocktail of serotonergic and dopaminergic agonists) (van den Brand et al., 2012), and even in some chronic SCI patients (Angeli et al., 2014; Harkema et al., 2011), While extensive axonal sprouting has been observed in these rats (van den Brand et al., 2012), it is unknown whether axon sprouting is causally related to the functional improvements. Recent studies suggest that electrical neuromodulation applied to the dorsal aspect of lumbar segments primarily engages proprioceptive feedback circuits (Capogrosso et al., 2013; Hofstoetter et al., 2015; Wenger et al., 2014). However, it remains unknown how this leads to functional restoration of descending input-dependent voluntary movement. In light of these results showing that reducing the excitability of inhibitory interneurons in the relay zone above the lumbar spinal cord is sufficient to enable this spinal circuitry to relay brain-derived commands to the lumbar spinal cord, it would be interesting to test whether epidural stimulation, and/or combined treatments, also engage such inhibitory interneurons to mediate their functional effects.

KCC2 and re-balancing spinal locomotor circuitry. Injury triggers a battery of alterations in the spinal cord, such as local KCC2 down-regulation. Results presented herein suggest that reactivation of KCC2 in inhibitory interneurons may re-establish the excitation/inhibition ratio (E/I ratio) across the spinal network following SCI. This is consistent with the notion that inhibitory input is critical not only for sculpting specific firing patterns within a neural network, but also for preventing network activity from becoming dysfunctional (Mohler et al., 2004). Importantly, not all inhibition-enhancing manipulations are effective. In contrast to KCC2 or Gi-DREADD, GABA receptor agonists appear to reduce the overall activation patterns across the spinal cord, but fail to re-establish more physiological activation patterns, or to promote functional improvements. This could be due to its direct and non-selective inhibition, as L838,417 treatment reduced neuronal activation levels in all spinal cord regions, including crucial ventral motor associated laminae, which is expected to decrease the quality of motor control overall. Finally, direct excitation of spinal excitatory interneurons failed to induce lasting functional recovery after SCI. Thus, instead of broadly targeting excitatory or inhibitory neurotransmission, fine-tuning the excitability of inhibitory interneurons appears to be a more effective strategy to make the spinal network receptive to both descending and sensory inputs for successful recovery of motor function.

Translational Perspectives. Based on a selective KCC2 activator identified from high-throughput screening, CLP290 has been optimized for systemic administration (Gagnon et al., 2013), and has been shown to effectively treat neuropathic pain in animal models (Ferrini et al., 2017; Gagnon et al., 2013). Unlike other compounds tested in this study, CLP290 exhibited negligible side effects even at high doses (data not shown). As the majority of SCI patients have some spared axons, these results suggest that this BBB-permeable small molecule, CLP290, could be a promising treatment in these cases. Despite this, not all aspects of hindlimb function were restored in these experiments. Thus, future studies should investigate the therapeutic effects of combining CLP290 with other treatments, such as additional rehabilitative training, on hindlimb recovery after SCI.

### Materials and Methods

TABLE 1
Key reagents used in experiments described herein.

| | | |
|---|---|---|
| REAGENT or RESOURCE | SOURCE | IDENTIFIER |

Antibodies

| | | |
|---|---|---|
| Chicken monoclonal anti-GFP | Abcam | Cat#ab13970 |
| Rabbit polyclonal anti-RFP | Abcam | Cat#ab34771 |
| Mouse monoclonal anti-NeuN | Millipore | Cat#MAB377 |
| Rabbit polyclonal anti-5-HT | Immunostar | Cat#20080 |
| Rat monoclonal anti-HA | Sigma | Cat#11867423001 |
| Rabbit polyclonal anti-GFAP | DAKO | Cat#Z0334 |
| Rabbit polyclonal anti-c-Fos | Cell signaling | Cat#2250s |
| Rabbit polyclonal anti-KCC2 | Milipore | Cat#07-432 |

Biological Samples

| | | |
|---|---|---|
| N/A | N/A | N/A |

Chemicals, Peptides, and Recombinant Proteins

| | | |
|---|---|---|
| Quipazine | Sigma | Cat#Q1004 |
| 8-OH-DPAT | Tocris | Cat#0529 |
| Clozapine N-oxide | Enzo Life | Cat#BML-NS105- |
| | Sciences | 0025 |
| Baclofen | Tocris | Cat#0417 |
| CP101606 | Sigma | Cat#SML0053 |
| CLP290 | PharmaBlock | Cat#N/A |
| L838,417 | PharmaBlock | Cat#PBLJ6533 |
| Bumetanide | Tocris | Cat#3108 |

Critical Commercial Assays

| | | |
|---|---|---|
| N/A | N/A | N/A |

Deposited Data

| | | |
|---|---|---|
| N/A | N/A | N/A |

Experimental Models: Cell Lines

| | | |
|---|---|---|
| N/A | N/A | N/A |

Experimental Models: Organisms/Strains

| | | |
|---|---|---|
| Mouse/C57B1/6 | Charles River | Strain code#027 |
| Mouse/Vgat-Cre | The Jackson | Jax#28862 |
| | Laboratory | |
| Mouse/Vglut2-Cre | The Jackson | Jax#28863 |
| | Laboratory | |
| Mouse/ChAT-Cre | The Jackson | Jax#28861 |
| | Laboratory | |

Recombinant DNA

| | | |
|---|---|---|
| AAV-syn-mCherry | This paper | Cat#N/A |
| AAV-syn-FLEX-HA-KCC2 | This paper | Cat#N/A |
| AAV-syn-FLEX-hM4Di-mCherry | Addgene | Cat#44362 |
| AAV-syn-FLEX-hM3Dq-mCherry | Addgene | Cat#44361 |
| AAV-CAG-FLEX-H2B-GFP | Vigenebio | Cat#N/A |
| AAV-CAG-H2B-GFP | This paper | Cat#N/A |
| AAV-CAG-GFP-WPRE | Wang et. al. 2017 | Cat#N/A |
| AAV-syn-HA-KCC2 | This paper | Cat#N/A |
| Lenti-HiRet-mCherry | Liu et al. 2017 | Cat#N/A |

Sequence-Based Reagents

| | | |
|---|---|---|
| N/A | N/A | N/A |

Software and Algorithms

| | | |
|---|---|---|
| Matlab 2017 | Mathworks | Found on the world wide web at |
| | | www.mathworks.com/ |
| ImageJ2 | NIH | Found on the world wide web at |
| | | https://imagej.nih.gov/ij/index.html |
| Simi | SIMI reality | Found on the world wide web at |
| | motion systems | www.simi.com/ |

Other

| | | |
|---|---|---|
| N/A | N/A | N/A |

Mouse Strains. All experimental procedures were performed in compliance with animal protocols approved by the Institutional Animal Care and Use Committee at Boston Children's Hospital. Mice employed in this study included: C57BL/6 wild-type (WT) mouse (Charles River, Strain code #027); and Vgat-Cre (Jax #28862), VGlut2-Cre (Jax #28863) and ChAT-Cre (Jax #28861) mouse strains maintained on C57BL/6 genetic background. For behavioral measurements, all experimental animals used were from different littermates. The 19-21 g adult female mice were randomized and assigned to different treatment groups, prior to injury, and no other specific randomization was used for the animal studies. Behavioral tests were examined blindly.

Chemicals and Antibodies. For systemic administration (i.p.): Quipazine [Sigma (Q1004), 0.2 mg/kg)] and 8-OH-DPAT [Tocris (0529), 0.1 mg/kg)] were suspended in 0.9% NaCl; Baclofen [Tocris (0417), 1 mg/kg)] was suspended in 100 mM NaOH and then 0.9% NaCl; CP101606 [Sigma (SML0053), 10 mg/kg)] was suspended in DMSO and then 0.9% NaCl; CLP290 [synthesized by PharmaBlock, 25 mg/kg] was suspended in DMSO and then 20% 2-hydroxypropyl-β-cyclodextrin; L838,417 [synthesized by PharmaBlock, 1 mg/kg] was suspended in 0.5% methylcellulose and 0.9% NaCl; and Bumetanide [Tocris, (3108), 0.3 mg/kg)] was suspended in 15% DMSO. For immunostaining and western blotting, the primary antibodies used were: chicken anti-GFP [Abcam (Cat: ab13970)], rabbit anti-RFP [Abcam (Cat: ab34771)], rabbit anti-GFAP [DAKO (Z0334)], rabbit anti-5-HT [Immunostar (20080)], rat anti-HA [Sigma (11867423001)], rabbit anti-c-Fos [Cell signaling (2250s)], mouse anti-NeuN [Millipore (MAB377)]; and rabbit anti-KCC2 [Milipore (07-432)].

Surgical Procedures. The procedure of T7 and T10 double lateral hemisection was similar to that described elsewhere (Courtine et al., 2008; van den Brand et al., 2012). Briefly, a midline incision was made over the thoracic vertebrae, followed by a T7-10 laminectomy. For the T7 right side over-hemisection, a scalpel and micro-scissors were carefully used to interrupt the bilateral dorsal column at T7, and ensured no sparing of ventral pathways on the contralateral side (FIG. 1A). For the T10 left hemisection, a scalpel and micro-scissors were carefully used to interrupt only the left side of the spinal cord until the midline. The muscle layers were then sutured, and the skin was secured with wound clips. All animals received post hoc histological analysis, and those with spared 5HT axons at the lumbar spinal cord (L2-5) were excluded for behavioral analysis (FIG. 7).

The procedure of T8 full transection was similar to that described elsewhere (Courtine et al., 2009). Briefly, a midline incision was made over the thoracic vertebrae, followed by a T8 laminectomy. The complete T8 transection was then performed carefully using both a scalpel and micro-scissors. The muscle layers were then sutured and the skin was secured with wound clips.

EMG Recording and cortical stimulation. The procedure for EMG recording in free moving animals was similar to that described previously (Pearson et al., 2005). In brief, at 9 weeks after surgery, 5 mice from each group (Control, CLP290 and AAV-KCC2 treated mice) underwent implantation of customized bipolar electrodes into selected hindlimb muscles to record EMG activity. Electrodes (793200, A-M Systems) were led by 30 gauge needles and inserted into the mid-belly of the medial gastrocnemius (GS) and tibialis anterior (TA) muscles of the right hindlimb. A common ground wire was inserted subcutaneously in the neck-shoulder area. Wires were routed subcutaneously through the back to a small percutaneous connector securely cemented to the skull of the mouse. EMG signals were acquired using a differential AC amplifier (1700, A-M Systems, WA) with 10-1000 Hz filtration, sampled at 4 kHz using a digitizer (PowerLab 16/35, ADInstruments), and analyzed by LabChart 8 (ADInstruments).

For epidural stimulation and EMG recording, a customized head plate was secured over the skull, and a monopolar stimulation electrode (SSM33A05, World Precision Instruments, Inc.) was positioned epidurally over the representative hindlimb area of left motor cortex. A train of electrical stimuli (0.2 ms biphasic pulse, 100 ms pulse train, 20 Hz, 0.5-1.5 mA) was generated by pulse generator and isolator (Master 9 and Iso-Flex, A.M.P.I.), and delivered during quadrupedal standing in fully awake condition. Testing was performed without and with electrochemical stimulations. Peak-to-peak amplitude and latency of evoked responses were computed from EMG recordings of the right TA muscle.

Virus Production and Injection. For the KCC2 overexpression virus injection procedure, AAV2/PHP.B-Syn-HA-KCC2 and AAV2/9-Syn-HA-KCC2 were injected into the tail vein of WT mice. AAV2/PHP.B-Syn-FLEX-HA-KCC2 was injected to Vgat-Cre, Vglut2-Cre and ChAT-Cre mice tail vein. AAV2/9-Syn-HA-KCC2 and AAV2/9-Syn-FLEX-HA-KCC2, AAV2/9-Syn-FLEX-hM4Di-mCherry and AV2/9-Syn-FLEX-hM3Dq-mCherry, were injected into WT, Vgat-Cre or Vglut2-cre mice tail vein. Tail vein virus injection was performed, as described previously (Deverman et al., 2016), 3 hours after SCI (AAV titers were adjusted to 4-5×10¹³ copies/ml for injection, produced by The Viral Core, Boston Children's Hospital). AAV2/1-Syn-HA-KCC2 and AAV2/1-Syn-FLEX-HA-KCC2 were intraspinally injected into the lumbar level (L2-4) of WT and Vgat-Cre mice, respectively. Lumbar level intraspinal virus injection was performed one day prior to SCI procedure, in order to eliminate any possible behaviorally defects caused by lumbar level intraspinal injection (AAV titers were adjusted to 0.5-1×10¹³ copies/ml for injection, produced by The Viral Core at Boston Children's Hospital).

For reticulospinal tracing experiments (procedure was described previously (Esposito et al., 2014)), AAV2/8-ChR2-YFP and AAV2/8-ChR2-mCherry were injected into the mouse right and left reticular formation in the brain stem respectively. For CST tracing experiments (procedure was described previously (Liu et al., 2010; Liu et al., 2017)), AAV2/8-ChR2-mCherry was injected to the mouse right sensorimotor cortex (all AAV titers were adjusted to 0.5-5×10¹³ copies/ml for injection, produced by The Viral Core, Boston Children's Hospital). For lumbar level retrograde tracing, vectors of HiRet-mCherry (lenti-virus titers were adjusted to 1.6-2×10¹² copies/ml for injection) were constructed based on the HiRet-lenti backbone (Kinoshita et al., 2012). Injection procedure was described previously (Wang et al., 2017), in which HiRet-mCherry is injected into left or right lumbar spinal cord from segments 2-4.

Immunohistochemistry and Imaging. The paraformaldehyde (PFA) fixed tissues were cryo-protected with 30% sucrose and processed using cryostat (section thickness 40 μm for spinal cord). Sections were treated with a blocking solution containing 10% normal donkey serum with 0.5% Triton-100 for 2 hours at room temperature before staining. The primary antibodies (4 □, overnight) used were: rabbit anti-GFAP [DAKO (Z0334), 1:600]; rabbit anti-5-HT [Immunostar (20080), 1:5,000]; chicken anti-GFP [Abcam (ab13970), 1:400]; rabbit anti-RFP [Abcam (ab34771), 1:400]; rabbit anti-PKCγ [Santa Cruz (sc211), 1:100]; rat anti-HA [Sigma (11867423001), 1:200]; rabbit anti-c-Fos [Cell signaling (2250s), 1:100]; and mouse anti-NeuN [Millipore (MAB377), 1:400]. Secondary antibodies (room temperature, 2 h) included: Alexa Fluor 488-conjugated donkey anti chicken and rabbit; and Alexa Fluor 594-conjugated donkey anti rabbit (all from Invitrogen). c-Fos immunoreactivity of spinal neurons was determined as previously described (Courtine et al., 2009), after 1-hour of continuous quadrupedal free walking (intact), stepping (CLP290 or AAV-KCC2 treated mice) or dragging (vehicle or AAV-GFP treated mice). The mice were returned to their cages, and were then anesthetized and sacrificed by intracardial perfusion of 4% PFA (wt/vol) in phosphate buffered saline (PBS) about 2 hours later.

Spinal cord transverse and horizontal sections were imaged with a confocal laser-scanning microscope (Zeiss 700 or Zeiss 710). To quantify and compare fluorescence intensity of: reticular spinal tract projections (RFP⁺ and GFP⁺), and corticospinal tract (CST) projections (GFP⁺), at different transverse spinal cord segments sections (FIGS. 10A and 10C); as well as 5HT axonal staining (FIG. 10B). All images, used for analysis under multiple conditions, were taken using the same optical parameters to avoid saturation. Densitometry measurements were taken by using FIJI software, after being sub-thresholded to the background and normalized by area.

aTo quantify and compare the retrograde HiRet-marked cell body of spinal neurons in different treatments, all images were decomposed to individual channels and planes. They were aligned and quantified using custom-developed MATLAB codes. HiRet-marked neurons were assigned coordinates manually.

Western Blotting. Animals were killed by decapitation after isoflurane anesthesia. Spinal cords were quickly dissected out from T5 to L1 and divided into 350 μm slices. Samples were homogenized in cold lysis buffer containing: 20 mmol/L Tris (pH 7.4), 125 mmol/L NaCl, 10% glycerol, 1% Triton X-100, 0.5% DCA, 0.1% SDS, 20 mmol/L NaF, 1 mmol/L phenylmethylsulfonyl fluoride, 4 μg/mL aprotinin, 4 μg/mL leupeptin, and 1 mmol/L Na3VO4. Then samples were centrifuged at 13,000 g for 10 minutes at 4° C. Protein concentrations in supernatant were assessed using the bicinchoninic acid protein assay kit (Bio-Rad, Hercules, Calif.). Equal amounts of protein extracts were resolved by 4-20% SDS-PAGE and electrotransferred onto polyvinylidene difluoride membranes (Millipore, Bedford, Mass.). After blockade in Tris-buffered saline plus 3% BSA, membranes were exposed to a polyclonal rabbit KCC2-specific antibody diluted 1 in 500 (Millipore), or a polyclonal rabbit beta-actin antibody diluted 1 in 2000 (cell signaling), in the blocking solution overnight at 4° C. ImmunoPure goat horseradish peroxidase-conjugated rabbit-specific antibodies were used (1 in 500 in blocking solution, 1 h at 22° C.) for chemiluminescent detection (Pierce Biotech).

Behavioral Experiments. Motor function was evaluated with a locomotor open field rating scale on the Basso Mouse Scale (BMS). For transient pharmacological treatments, ten to fifteen minutes (van den Brand et al., 2012) prior to behavioral tests (grounding walking, all of which were performed individually), mice received systematic administration (i.p.) of the neural modulators listed above. It is important to note that with a single intraperitoneal injection, plasma CNO levels peak at 15 min and become very low by 2 h after injection (Guettier et al., 2009). For chronic pharmacological treatments, 24 hours prior to behavioral tests, mice received systematic administration of the compounds listed above. All behavioral tests were completed within 1-3 hours. For detailed hindlimb kinematic analysis, mice from different groups were placed in the MotoRater (TSE Systems, (Zorner et al., 2010)), and all kinematic analysis was performed based on data collected by the MotoRater.

QUANTIFICATION AND STATISTICAL ANALYSIS. The normality and variance similarity were measured by STATA (version 12, College station, TX, USA) before any parametric tests were applied. Two-tailed student's t-test was used for the single comparison between two groups. The rest of the data were analyzed using one-way or two-way ANOVA depending on the appropriate design. Post hoc comparisons were carried out only when the primary measure showed statistical significance. P-value of multiple comparisons was adjusted by using Bonferroni's correction. Error bars in all figures represent mean±S.E.M. The mice with different litters, body weights and sexes were randomized and assigned to different treatment groups, and no other specific randomization was used for the animal studies.

### REFERENCES

Angeli, C. A., Edgerton, V. R., Gerasimenko, Y. P., and Harkema, S. J. (2014). Altering spinal cord excitability enables voluntary movements after chronic complete paralysis in humans. Brain 137, 1394-1409.
Arber, S. (2012). Motor circuits in action: specification, connectivity, and function. Neuron 74, 975-989, 2012.
Ballermann, M., and Fouad, K. (2006). Spontaneous locomotor recovery in spinal cord injured rats is accompanied by anatomical plasticity of reticulospinal fibers. Eur J Neurosci 23, 1988-1996.
Bareyre, F. M., Kerschensteiner, M., Raineteau, O., Mettenleiter, T. C., Weinmann, O., and Schwab, M. E. (2004). The injured spinal cord spontaneously forms a new intraspinal circuit in adult rats. Nat Neurosci 7, 269-277.
Basso, D. M., Fisher, L. C., Anderson, A. J., Jakeman, L. B., McTigue, D. M., and Popovich, P. G. (2006). Basso Mouse Scale for locomotion detects differences in recovery after spinal cord injury in five common mouse strains. J Neurotrauma 23, 635-659.
Ben-Ari, Y., Woodin, M. A., Sernagor, E., Cancedda, L., Vinay, L., Rivera, C., Legendre, P., Luhmann, H. J., Bordey, A., Wenner, P., et al. (2012). Refuting the challenges of the developmental shift of polarity of GABA actions: GABA more exciting than ever! Front Cell Neurosci 6, 35.
Boulenguez, P., Liabeuf, S., Bos, R., Bras, H., Jean-Xavier, C., Brocard, C., Stil, A., Darbon, P., Cattaert, D., Delpire, E., et al. (2010). Down-regulation of the potassium-chloride cotransporter KCC2 contributes to spasticity after spinal cord injury. Nat Med 16, 302-307.
Capogrosso, M., Wenger, N., Raspopovic, S., Musienko, P., Beauparlant, J., Bassi Luciani, L., Courtine, G., and Micera, S. (2013). A computational model for epidural electrical stimulation of spinal sensorimotor circuits. J Neurosci 33, 19326-19340.
Cazalets, J. R., Sqalli-Houssaini, Y., and Clarac, F. (1992). Activation of the central pattern generators for locomotion by serotonin and excitatory amino acids in neonatal rat. J Physiol 455, 187-204.
Cote, M. P., Gandhi, S., Zambrotta, M., and Houle, J. D. (2014). Exercise modulates chloride homeostasis after spinal cord injury. J Neurosci 34, 8976-8987.
Courtine, G., Gerasimenko, Y., van den Brand, R., Yew, A., Musienko, P., Zhong, H., Song, B., Ao, Y., Ichiyama, R. M., Lavrov, I., et al. (2009). Transformation of nonfunctional spinal circuits into functional states after the loss of brain input. Nat Neurosci 12, 1333-1342.
Courtine, G., Song, B., Roy, R. R., Zhong, H., Herrmann, J. E., Ao, Y., Qi, J., Edgerton, V. R., and Sofroniew, M. V. (2008). Recovery of supraspinal control of stepping via indirect propriospinal relay connections after spinal cord injury. Nat Med 14, 69-74.
Cowley, K. C., and Schmidt, B. J. (1995). Effects of inhibitory amino acid antagonists on reciprocal inhibitory interactions during rhythmic motor activity in the in vitro neonatal rat spinal cord. J Neurophysiol 74, 1109-1117.
de Leon, R. D., Tamaki, H., Hodgson, J. A., Roy, R. R., and Edgerton, V. R. (1999). Hindlimb locomotor and postural training modulates glycinergic inhibition in the spinal cord of the adult spinal cat. J Neurophysiol 82, 359-369.
Deverman, B. E., Pravdo, P. L., Simpson, B. P., Kumar, S. R., Chan, K. Y., Banerjee, A., Wu, W. L., Yang, B., Huber, N., Pasca, S. P., et al. (2016). Cre-dependent selection yields AAV variants for widespread gene transfer to the adult brain. Nat Biotechnol 34, 204-209.
Doyon, N., Vinay, L., Prescott, S. A., and De Koninck, Y. (2016). Chloride Regulation: A Dynamic Equilibrium Crucial for Synaptic Inhibition. Neuron 89, 1157-1172.
Edgerton, V. R., Courtine, G., Gerasimenko, Y. P., Lavrov, I., Ichiyama, R. M., Fong, A. J., Cai, L. L., Otoshi, C. K., Tillakaratne, N. J., Burdick, J. W., et al. (2008). Training locomotor networks. Brain Res Rev 57, 241-254.
Edgerton, V. R., Tillakaratne, N. J., Bigbee, A. J., de Leon, R. D., and Roy, R. R. (2004). Plasticity of the spinal neural circuitry after injury. Annu Rev Neurosci 27, 145-167.
Esposito, M. S., Capelli, P., and Arber, S. (2014). Brainstem nucleus MdV mediates skilled forelimb motor tasks. Nature 508, 351-356.
Fawcett, J. W., Curt, A., Steeves, J. D., Coleman, W. P., Tuszynski, M. H., Lammertse, D., Bartlett, P. F., Blight, A. R., Dietz, V., Ditunno, J., et al. (2007). Guidelines for the conduct of clinical trials for spinal cord injury as developed by the ICCP panel: spontaneous recovery after spinal cord injury and statistical power needed for therapeutic clinical trials. Spinal Cord 45, 190-205.
Ferrini, F., Lorenzo, L. E., Godin, A. G., Quang, M. L., and De Koninck, Y. (2017). Enhancing KCC2 function counteracts morphine-induced hyperalgesia. Sci Rep 7, 3870.
Filous, A. R., and Schwab, J. M. (2017). Determinants of Axon Growth, Plasticity, and Regeneration in the Context of Spinal Cord Injury. Am J Pathol. 188, 53-62.
Frigon, A., and Rossignol, S. (2008). Adaptive changes of the locomotor pattern and cutaneous reflexes during locomotion studied in the same cats before and after spinalization. J Physiol 586, 2927-2945.
Gackiere, F., and Vinay, L. (2015). Contribution of the potassium-chloride cotransporter KCC2 to the strength of inhibition in the neonatal rodent spinal cord in vitro. J Neurosci 35, 5307-5316.
Gagnon, M., Bergeron, M. J., Lavertu, G., Castonguay, A., Tripathy, S., Bonin, R. P., Perez-Sanchez, J., Boudreau, D., Wang, B., Dumas, L., et al. (2013). Chloride extrusion enhancers as novel therapeutics for neurological diseases. Nat Med 19, 1524-1528.
Gerasimenko, Y., Roy, R. R., and Edgerton, V. R. (2008). Epidural stimulation: comparison of the spinal circuits that generate and control locomotion in rats, cats and humans. Exp Neurol 209, 417-425.
Grillner, S., and Wallen, P. (1985). Central pattern generators for locomotion, with special reference to vertebrates. Annu Rev Neurosci 8, 233-261.
Guettier, J. M., Gautam, D., Scarselli, M., Ruiz de Azua, I., Li, J. H., Rosemond, E., Ma, X., Gonzalez, F. J., Armbruster, B. N., Lu, H., et al. (2009). A chemical-genetic approach to study G protein regulation of beta cell function in vivo. Proc Natl Acad Sci USA 106, 19197-19202.
Harkema, S., Gerasimenko, Y., Hodes, J., Burdick, J., Angeli, C., Chen, Y., Ferreira, C., Willhite, A., Rejc, E., Grossman, R. G., et al. (2011). Effect of epidural stimulation of the lumbosacral spinal cord on voluntary movement, standing, and assisted stepping after motor complete paraplegia: a case study. Lancet 377, 1938-1947.
He, Z., and Jin, Y. (2016). Intrinsic control of axon regeneration. Neuron 90, 437-451. Hofstoetter, U. S., Danner, S. M., Freundl, B., Binder, H., Mayr, W., Rattay, F., and Minassian, K. (2015). Periodic modulation of repetitively elicited monosynaptic reflexes of the human lumbosacral spinal cord. J Neurophysiol 114, 400-410.
Hubner, C. A., Stein, V., Hermans-Borgmeyer, I., Meyer, T., Ballanyi, K., and Jentsch, T. J. (2001). Disruption of KCC2 reveals an essential role of K—Cl cotransport already in early synaptic inhibition. Neuron 30, 515-524.
Jankowska, E., and Edgley, S. A. (2006). How can corticospinal tract neurons contribute to ipsilateral movements? A question with implications for recovery of motor functions. Neuroscientist 12, 67-79.
Kaila, K., Ruusuvuori, E., Sej a, P., Voipio, J., and Puskarjov, M. (2014). GABA actions and ionic plasticity in epilepsy. Curr Opin Neurobiol 26, 34-41.
Kakulas, B. A. (1999). A review of the neuropathology of human spinal cord injury with emphasis on special features. J Spinal Cord Med 22, 119-124.
Kato, S., Kobayashi, K., Inoue, K., Kuramochi, M., Okada, T., Yaginuma, H., Morimoto, K., Shimada, T., Takada, M., and Kobayashi, K. (2011). A lentiviral strategy for highly efficient retrograde gene transfer by pseudotyping with fusion envelope glycoprotein. Hum Gene Ther 22, 197-206.
Kiehn, 0. (2006). Locomotor circuits in the mammalian spinal cord. Annu Rev Neurosci 29, 279-306.
Kiehn, 0. (2016). Decoding the organization of spinal circuits that control locomotion. Nat Rev Neurosci 17, 224-238.
Kinoshita, M., Matsui, R., Kato, S., Hasegawa, T., Kasahara, H., Isa, K., Watakabe, A., Yamamori, T., Nishimura, Y., Alstermark, B., et al. (2012). Genetic dissection of the circuit for hand dexterity in primates. Nature 487, 235-238.
Knabl, J., Witschi, R., Hosl, K., Reinold, H., Zeilhofer, U. B., Ahmadi, S., Brockhaus, J., Sergejeva, M., Hess, A., Brune, K., et al. (2008). Reversal of pathological pain through specific spinal GABAA receptor subtypes. Nature 451, 330-334.
Krashes, M. J., Koda, S., Ye, C., Rogan, S. C., Adams, A. C., Cusher, D. S., Maratos-Flier, E., Roth, B. L., and Lowell, B. B. (2011). Rapid, reversible activation of AgRP neurons drives feeding behavior in mice. J Clin Invest 121, 1424-1428.
Liu, K., Lu, Y., Lee, J. K., Samara, R., Willenberg, R., Sears-Kraxberger, I., Tedeschi, A., Park, K. K., Jin, D., Cai, B., et al. (2010). PTEN deletion enhances the regenerative ability of adult corticospinal neurons. Nat Neurosci 13, 1075-1081.
Liu, Y., Wang, X., Li, W., Zhang, Q., Li, Y., Zhang, Z., Zhu, J., Chen, B., Williams, P. R., Zhang, Y., et al. (2017). A Sensitized IGF1 Treatment Restores Corticospinal Axon-Dependent Functions. Neuron 95, 817-833 e814.
Lu, Y., Zheng, J Xiong, L., Zimmermann, M., and Yang, J. (2008). Spinal cord injury-induced attenuation of GABAergic inhibition in spinal dorsal horn circuits is associated with down-regulation of the chloride transporter KCC2 in rat. J Physiol 586, 5701-5715.
Mohler, H., Fritschy, J. M., Crestani, F., Hensch, T., and Rudolph, U. (2004). Specific GABA(A) circuits in brain development and therapy. Biochem Pharmacol 68, 1685-1690.
Ni, Y., Nawabi, H., Liu, X., Yang, L., Miyamichi, K., Tedeschi, A., Xu, B., Wall, N. R., Callaway, E. M., and He, Z. (2014). Characterization of long descending premotor propriospinal neurons in the spinal cord. J Neurosci 34, 9404-9417.
O'Shea, T. M., Burda, J. E., and Sofroniew, M. V. (2017). Cell biology of spinal cord injury and repair. J Clin Invest 127, 3259-3270.
Pearson, K. G., Acharya, H., and Fouad, K. (2005). A new electrode configuration for recording electromyographic activity in behaving mice. J Neurosci Methods 148, 36-42.
Robinson, G. A., and Goldberger, M. E. (1986). The development and recovery of motor function in spinal cats. II. Pharmacological enhancement of recovery. Exp Brain Res 62, 387-400.
Rosenzweig, E. S., Courtine, G., Jindrich, D. L., Brock, J. H., Ferguson, A. R., Strand, S. C., Nout, Y. S., Roy, R. R., Miller, D. M., Beattie, M. S., et al. (2010). Extensive spontaneous plasticity of corticospinal projections after primate spinal cord injury. Nat Neurosci 13, 1505-1510.
Rossi, J., Balthasar, N., Olson, D., Scott, M., Berglund, E., Lee, C. E., Choi, M. J., Lauzon, D., Lowell, B. B., and Elmquist, J. K. (2011). Melanocortin-4 receptors expressed by cholinergic neurons regulate energy balance and glucose homeostasis. Cell Metab 13, 195-204.
Rossignol, S., and Barbeau, H. (1993). Pharmacology of locomotion: an account of studies in spinal cats and spinal cord injured subjects. J Am Paraplegia Soc 16, 190-196.
Rossignol, S., and Frigon, A. (2011). Recovery of locomotion after spinal cord injury: some facts and mechanisms. Annu Rev Neurosci 34, 413-440.
Ruder, L., Takeoka, A., and Arber, S. (2016). Long-Distance Descending Spinal Neurons Ensure Quadrupedal Locomotor Stability. Neuron 92, 1063-1078.
Sawada, M., Kato, K., Kunieda, T., Mikuni, N., Miyamoto, S., Onoe, H., Isa, T., and Nishimura, Y. (2015). Function of the nucleus accumbens in motor control during recovery after spinal cord injury. Science 350, 98-101.
Schucht, P., Raineteau, O., Schwab, M. E., and Fouad, K. (2002). Anatomical correlates of locomotor recovery following dorsal and ventral lesions of the rat spinal cord. Exp Neurol 176, 143-153.
Stepien, A. E., Tripodi, M., Arber, S. (2010). Monosynaptic rabies virus reveals premotor network organization and synaptic specificity of cholinergic partition cells. Neuron 68, 456-472.
Takeoka, A., Vollenweider, I., Courtine, G., and Arber, S. (2014). Muscle spindle feedback directs locomotor recovery and circuit reorganization after spinal cord injury. Cell 159, 1626-1639.
Tong, Q., Ye, C., McCrimmon, R. J., Dhillon, H., Choi, B., Kramer, M. D., Yu, J., Yang, Z., Christiansen, L. M., Lee, C. E., et al. (2007). Synaptic glutamate release by ventromedial hypothalamic neurons is part of the neurocircuitry that prevents hypoglycemia. Cell Metab 5, 383-393.
van den Brand, R., Heutschi, J., Barraud, Q., DiGiovanna, J., Bartholdi, K., et al. (2012). Restoring voluntary control of locomotion after paralyzing spinal cord injury. Science 336, 1182-1185.
Vong, L., Ye, C., Yang, Z., Choi, B., Chua, S., Jr., and Lowell, B. B. (2011). Leptin action on GABAergic neurons prevents obesity and reduces inhibitory tone to POMC neurons. Neuron 71, 142-154.
Wang, X., Liu, Y., Li, X., Zhang, Z., Yang, H., Zhang, Y., Williams, P. R., Alwahab, N. S. A., Kapur, K., Yu, B., et al. (2017). Deconstruction of Corticospinal Circuits for Goal-Directed Motor Skills. Cell 171, 440-455 e414.
Wenger, N., Moraud, E. M., Raspopovic, S., Bonizzato, M., DiGiovanna, J., et al. (2014). Closed-loop neuromodulation of spinal sensorimotor circuits controls refined locomotion after complete spinal cord injury. Sci Transl Med 6, 255ra133.
Zaporozhets, E., Cowley, K. C., and Schmidt, B. J. (2011). Neurochemical excitation of propriospinal neurons facilitates locomotor command signal transmission in the lesioned spinal cord. J Neurophysiol 105, 2818-2829.
Zorner, B., Filli, L., Starkey, M. L., Gonzenbach, R., Kasper, H., Rothlisberger, M., Bolliger, M., and Schwab, M. E. (2010). Profiling locomotor recovery: comprehensive quantification of impairments after CNS damage in rodents. Nat Methods 7, 701-708.

1. A method for promoting functional recovery after paralysis, comprising administering to a subject having a central nervous system (CNS) lesion an effective amount of an agent that increases neuron-specific K⁺—Cl⁻ co-transporter (KCC2) expression and/or activity.

2. The method of claim 1, wherein the agent that increases KCC2 expression and/or activity is selected from the group consisting of a small molecule, a peptide, a gene editing system, and an expression vector encoding KCC2.

3-10. (canceled)

11. The method of claim 1, wherein the CNS lesion is a spinal injury.

12. The method of claim 1, wherein the subject is human.

13. The method of claim 1, wherein the subject has been diagnosed with a spinal injury.

14-16. (canceled)

17. The method of claim 1, wherein the subject is further administered at least a second therapeutic compound.

18. The method of claim 17, wherein the second therapeutic compound is selected from the group consisting of osteopontin, a growth factor, and 4-aminopuridine.

19. A method for promoting functional recovery after paralysis, comprising administering to a subject having a central nervous system (CNS) lesion an effective amount of an agent that inhibits Na⁺/2Cl⁻/K⁺ co-transporter (NKCC) expression and/or activity.

20. The method of claim 19, wherein the agent that inhibits NKCC expression and/or activity is selected from the group consisting of a small molecule, an antibody, a peptide, an antisense oligonucleotide, and an RNAi.

21. The method of claim 20, wherein the RNAi is a microRNA, an siRNA, or an shRNA.

22. The method of claim 20, wherein the small molecule is bumetanide.

23-32. (canceled)

33. A method for promoting functional recovery after paralysis, comprising administering to a subject having a central nervous system (CNS) lesion an effective amount of electrical stimulation that reduces excitability of inhibitory interneurons.

34-39. (canceled)

40. A pharmaceutical composition comprising
an effective amount of a KCC2 polypeptide or a vector comprising a nucleic acid sequence encoding the KCC2 polypeptide;
a pharmaceutically acceptable carrier.

41-66. (canceled)

67. The method of claim 2, wherein the expression vector encoding KCC2 is a non-integrative or integrative vector.

68. The method of claim 2, wherein the expression vector encoding KCC2 is a viral vector or a non-viral vector.

69. The method of claim 68, wherein the viral vector is selected from the group consisting of a retrovirus, lentivirus, adenovirus, herpesvirus, poxvirus, alpha virus, vaccinia virus, and adeno-associated virus (AAV).

70. The method of claim 69, wherein the AAV comprises an AAV9 capsid.

71. The method of claim 70, wherein the KCC2 is operably linked to a human synapsin promoter.

72. The method of claim 11, wherein the spinal injury is a severe spinal cord injury.


SEQUENCE LISTING (5 sequences)

SEQ ID NO: 1 - DNA, 3351 bp, Rattus sp.
atgctcaacaacctgacggactgcgaggacggcgatgggggagccaacccgggtgacggcaatcccaaggagagcagccccttcatcaacagcacggacacggagaaggggagagagtatgatggcaggaacatggccctgtttgaggaggagatggacaccagccccatggtatcctccctgctcagtgggctggccaactacaccaacctgcctcagggaagcaaagagcacgaagaagcagaaaacaatgagggcggaaagaagaagccggtgcaggccccacgcatgggcaccttcatgggcgtgtacctcccgtgcctgcagaacatctttggtgttatcctctttctgcggctcacttgggtggtgggaatcgcaggcatcatggagtccttctgcatggtcttcatctgctgctcctgcacgatgctcacagccatttccatgagcgcaattgcaaccaatggtgttgtgcctgctggtggctcctactacatgatttccaggtctctgggcccggagtttgggggcgccgtgggcctctgcttctacctgggcactacctttgctggggctatgtacatcctgggcaccatcgagatcctgctggcttacctcttcccagcgatggccatcttcaaggcagaagatgccagtggggaggcagccgccatgttgaataacatgcgggtgtatggcacctgtgtgctcacctgcatggccaccgtagtctttgtgggcgtcaagtacgtgaacaagtttgccctggtcttcctgggttgcgtgatcctctccatcctggccatctacgcaggggtcatcaagtctgccttcgatccacccaatttcccgatttgcctcctggggaaccgcacgctgtctcgccatggctttgatgtctgtgccaagctggcttgggaaggaaatgagacagtgaccacacggctctggggcctattctgttcctcccgcctcctcaatgccacctgtgatgagtacttcacccgaaacaatgtcacagagatccagggcattcctggtgctgcaagtggcctcatcaaagagaacctgtggagttcctacctgaccaagggggtgatcgtggagaggcgtgggatgccctctgtgggcctggcagatggtacccccgttgacatggaccacccctatgtcttcagtgatatgacctcctacttcaccctgcttgttggcatctatttcccctcagtcacagggatcatggctggctcgaaccggtccggagacctgcgggatgcccagaagtctatccctactggaactatcttggccattgctacgacctctgctgtctacatcagctctgttgttctgttcggagcctgcatcgaaggggtcgtcctacgggacaagtttggggaagctgtgaatggcaatctggtggtgggcaccctggcctggccttctccttgggtcattgtcataggctctttcttctctacctgcggagctggactacagagcctcacaggggccccacgcctgctgcaggccatctcccgggatggcatagtgcccttcctgcaggtctttggccatggcaaagccaacggagagccaacctgggcgctgctgctgactgcctgcatctgtgagatcggcatcctcatcgcctccctggatgaggtcgcccctatcctttccatgttcttcctgatgtgttacatgtttgtgaacttggcttgcgcggtgcagacactgctgaggacgcccaactggaggccacgcttccgatattaccactggaccctctccttcctgggcatgagcctctgcctggccctgatgttcatttgctcctggtattatgcgctggtagctatgctcatcgctggcctcatctataagtacatcgagtaccggggggcagagaaggagtggggggatgggatccgaggcctgtctctcagtgcagctcgctatgctctcttgcgtctggaggaaggacccccgcatacaaagaactggaggccccagctactggtgctggtgcgtgtggaccaggaccagaacgtggtgcacccgcagctgctgtccttgacctcccagctcaaggcagggaagggcctgaccattgtgggctctgtccttgagggcacctttctggacaaccaccctcaggctcagcgggcagaggagtctatccggcgcctgatggaggctgagaaggtgaagggcttctgccaggtagtgatctcctccaacctgcgtgacggtgtgtcccacctgatccaatccgggggcctcgggggcctgcaacacaacactgtgctagtgggctggcctcgcaactggcgacagaaggaggatcatcagacatggaggaacttcatcgaactcgtccgggaaactacagctggccacctcgccctgctggtcaccaagaatgtttccatgttccccgggaaccctgagcgtttctctgagggcagcattgacgtgtggtggatcgtgcacgacgggggcatgctcatgctgttgcccttcctcctgcgtcaccacaaggtctggaggaaatgcaaaatgcggatcttcaccgtggcgcagatggatgacaacagcattcagatgaagaaagacctgaccacgtttctgtaccacttacgaattactgcagaggtggaagtcgtggagatgcacgagagcgacatctcagcatacacctacgagaagacattggtaatggaacaacgttctcagatcctcaaacagatgcacctcaccaagaacgagcgggaacgggagatccagagcatcacagatgaatctcggggctccattcggaggaagaatccagccaacactcggctccgcctcaatgttcccgaagagacagcttgtgacaacgaggagaagccagaagaggaggtgcagctgatccatgaccagagtgctcccagctgccctagcagctcgccgtctccaggggaggagcctgagggggagggggagacagacccagagaaggtgcatctcacctggaccaaggataagtcagcggctcagaagaacaaaggccccagtcccgtctcctcggaggggatcaaggacttcttcagcatgaagccggagtgggaaaacttgaaccagtccaacgtgcggcgcatgcacacagctgtgcggctgaacgaggtcatcgtgaataaatcccgggatgccaagttggtgttgctcaacatgcccgggcctccccgcaaccgcaatggagatgaaaactacatggagttcctggaggtcctcactgagcaactggaccgggtgatgctggtccgcggtggtggccgagaggtcatcaccatctactcctga

SEQ ID NO: 2 - DNA, 1439 bp, Artificial Sequence
  Description of Artificial Sequence Synthetic polynucleotide
atggccaacttcacacctgtcaatggcagctcgggcaatcagtccgtgcgcctggtcacgtcatcatcccacaatcgctatgagacggtggaaatggtcttcattgccacagtgacaggctccctgagcctggtgactgtcgtgggcaacatcctggtgatgctgtccatcaaggtcaacaggcagctgcagacagtcaacaactacttcctcttcagcctggcgtgtgctgatctcatcataggcgccttctccatgaacctctacaccgtgtacatcatcaagggctactggcccctgggcgccgtggtctgcgacctgtggctggccctggactgcgtggtgagcaacgcctccgtcatgaaccttctcatcatcagctttgaccgctacttctgcgtcaccaagcctctcacctaccctgcccggcgcaccaccaagatggcaggcctcatgattgctgctgcctgggtactgtccttcgtgctctgggcgcctgccatcttgttctggcagtttgtggtgggtaagcggacggtgcccgacaaccagtgcttcatccagttcctgtccaacccagcagtgacctttggcacagccattgctggcttctacctgcctgtggtcatcatgacggtgctgtacatccacatctccctggccagtcgcagccgagtccacaagcaccggcccgagggcccgaaggagaagaaagccaagacgctggccttcctcaagagcccactaatgaagcagagcgtcaagaagcccccgcccggggaggccgcccgggaggagctgcgcaatggcaagctggaggaggcccccccgccagcgctgccaccgccaccgcgccccgtggctgataaggacacttccaatgagtccagctcaggcagtgccacccagaacaccaaggaacgcccagccacagagctgtccaccacagaggccaccacgcccgccatgcccgcccctcccctgcagccgcgggccctcaacccagcctccagatggtccaagatccagattgtgacgaagcagacaggcaatgagtgtgtgacagccattgagattgtgcctgccacgccggctggcatgcgccctgcggccaacgtggcccgcaagttcgccagcatcgctcgcaaccaggtgcgcaagaagcggcagatggcggcccgggagcgcaaagtgacacgaacgatctttgccattctgctggccttcatcctcacctggacgccctacaacgtcatggtcctggtgaacaccttctgccagagctgcatccctgacacggtgtggtccattggctactggctctgctacgtcaacagcaccatcaaccctgcctgctatgctctgtgcaacgccacctttaaaaagaccttccggcacctgctgctgtgccagtatcggaacatcggcactgccaggcg

SEQ ID NO: 3 - protein, 427 aa, Homo sapiens
MGSVRTNRYSIVSSEEDGMKLATMAVANGFGNGKSKVHTRQQCRSRFVKKDGHCNVQFINVGEKGQRYLADIFTTCVDIRWRWMLVIFCLAFVLSWLFFGCVFWLIALLHGDLDASKEGKACVSEVNSFTAAFLFSIETQTTIGYGFRCVTDECPIAVFMVVFQSIVGCIIDAFIIGAVMAKMAKPKKRNETLVFSHNAVIAMRDGKLCLMWRVGNLRKSHLVEAHVRAQLLKSRITSEGEYIPLDQIDINVGFDSGIDRIFLVSPITIVHEIDEDSPLYDLSKQDIDNADFEIVVILEGMVEATAMTTQCRSSYLANEILWGHRYEPVLFEEKHYYKVDYSRFHKTYEVPNTPLCSARDLAEKKYILSNANSFCYENEVALTSKEEDDSENGVPESTSTDTPPDIDLHNQASVPLEPRPLRRESEI

SEQ ID NO: 4 - DNA, 3639 bp, Homo sapiens
atggagccgcggcccacggcgccctcctccggcgccccgggactggccggggtcggggagacgccgtcagccgctgcgctggccgcagccagggtggaactgcccggcacggctgtgccctcggtgccggaggatgctgcgcccgcgagccgggacggcggcggggtccgcgatgagggccccgcggcggccggggacgggctgggcagacccttggggcccaccccgagccagagccgtttccaggtggacctggtttccgagaacgccgggcgggccgctgctgcggcggcggcggcggcggcggcagcggcggcggctggtgctggggcgggggccaagcagacccccgcggacggggaagccagcggcgagagcgagccggctaaaggcagcgaggaagccaagggccgcttccgcgtgaacttcgtggacccagctgcctcctcgtcggctgaagacagcctgtcagatgctgccggggtcggagtcgacgggcccaacgtgagcttccagaacggcggggacacggtgctgagcgagggcagcagcctgcactccggcggcggcggcggcagtgggcaccaccagcactactattatgatacccacaccaacacctactacctgcgcaccttcggccacaacaccatggacgctgtgcccaggatcgatcactaccggcacacagccgcgcagctgggcgagaagctgctccggcctagcctggcggagctccacgacgagctggaaaaggaaccttttgaggatggctttgcaaatggggaagaaagtactccaaccagagatgctgtggtcacgtatactgcagaaagtaaaggagtcgtgaagtttggctggatcaagggtgtattagtacgttgtatgttaaacatttggggtgtgatgcttttcattagattgtcatggattgtgggtcaagctggaataggtctatcagtccttgtaataatgatggccactgttgtgacaactatcacaggattgtctacttcagcaatagcaactaatggatttgtaagaggaggaggagcatattatttaatatctagaagtctagggccagaatttggtggtgcaattggtctaatcttcgcctttgccaacgctgttgcagttgctatgtatgtggttggatttgcagaaaccgtggtggagttgcttaaggaacattccatacttatgatagatgaaatcaatgatatccgaattattggagccattacagtcgtgattcttttaggtatctcagtagctggaatggagtgggaagcaaaagctcagattgttcttttggtgatcctacttcttgctattggtgatttcgtcataggaacatttatcccactggagagcaagaagccaaaagggttttttggttataaatctgaaatatttaatgagaactttgggcccgattttcgagaggaagagactttcttttctgtatttgccatcttttttcctgctgcaactggtattctggctggagcaaatatctcaggtgatcttgcagatcctcagtcagccatacccaaaggaacactcctagccattttaattactacattggtttacgtaggaattgcagtatctgtaggttcttgtgttgttcgagatgccactggaaacgttaatgacactatcgtaacagagctaacaaactgtacttctgcagcctgcaaattaaactttgatttttcatcttgtgaaagcagtccttgttcctatggcctaatgaacaacttccaggtaatgagtatggtgtcaggatttacaccactaatttctgcaggtatattttcagccactctttcttcagcattagcatccctagtgagtgctcccaaaatatttcaggctctatgtaaggacaacatctacccagctttccagatgtttgctaaaggttatgggaaaaataatgaacctcttcgtggctacatcttaacattcttaattgcacttggattcatcttaattgctgaactgaatgttattgcaccaattatctcaaacttcttccttgcatcatatgcattgatcaatttttcagtattccatgcatcacttgcaaaatctccaggatggcgtcctgcattcaaatactacaacatgtggatatcacttcttggagcaattctttgttgcatagtaatgttcgtcattaactggtgggctgcattgctaacatatgtgatagtccttgggctgtatatttatgttacctacaaaaaaccagatgtgaattggggatcctctacacaagccctgacttacctgaatgcactgcagcattcaattcgtctttctggagtggaagaccacgtgaaaaactttaggccacagtgtcttgttatgacaggtgctccaaactcacgtccagctttacttcatcttgttcatgatttcacaaaaaatgttggtttgatgatctgtggccatgtacatatgggtcctcgaagacaagccatgaaagagatgtccatcgatcaagccaaatatcagcgatggcttattaagaacaaaatgaaggcattttatgctccagtacatgcagatgacttgagagaaggtgcacagtatttgatgcaggctgctggtcttggtcgtatgaagccaaacacacttgtccttggatttaagaaagattggttgcaagcagatatgagggatgtggatatgtatataaacttatttcatgatgcttttgacatacaatatggagtagtggttattcgcctaaaagaaggtctggatatatctcatcttcaaggacaagaagaattattgtcatcacaagagaaatctcctggcaccaaggatgtggtagtaagtgtggaatatagtaaaaagtccgatttagatacttccaaaccactcagtgaaaaaccaattacacacaaagttgaggaagaggatggcaagactgcaactcaaccactgttgaaaaaagaatccaaaggccctattgtgcctttaaatgtagctgaccaaaagcttcttgaagctagtacacagtttcagaaaaaacaaggaaagaatactattgatgtctggtggctttttgatgatggaggtttgaccttattgataccttaccttctgacgaccaagaaaaaatggaaagactgtaagatcagagtattcattggtggaaagataaacagaatagaccatgaccggagagcgatggctactttgcttagcaagttccggatagacttttctgatatcatggttctaggagatatcaataccaaaccaaagaaagaaaatattatagcttttgaggaaatcattgagccatacagacttcatgaagatgataaagagcaagatattgcagataaaatgaaagaagatgaaccatggcgaataacagataatgagcttgaactttataagaccaagacataccggcagatcaggttaaatgagttattaaaggaacattcaagcacagctaatattattgtcatgagtctcccagttgcacgaaaaggtgctgtgtctagtgctctctacatggcatggttagaagctctatctaaggacctaccaccaatcctcctagttcgtgggaatcatcagagtgtccttaccttctattcataa

SEQ ID NO: 5 - protein, 428 aa, Mus sp. [sequence omitted]